(19) United States
(12) Patent Application Publication
(10) Pub. No.: US 2021/0355546 A1
(43) Pub. Date: Nov. 18, 2021

(54) Methods to Diagnose and Treat Cancer Using Non-Human Nucleic Acids

(71) Applicant: The Regents of the University of California, Oakland, CA (US)

(72) Inventors: Gregory D. Poore, La Jolla, CA (US); Robin Knight, La Jolla, CA (US)

(21) Appl. No.: 17/286,083

(22) PCT Filed: Nov. 4, 2019
(86) PCT No.: PCT/US2019/059647

§ 371 Date: Apr. 16, 2021

Related U.S. Application Data
(60) Provisional application No. 62/754,696, filed on Nov. 2, 2018.

(51) Int. Cl. C12Q 1/6886

(52) U.S. Cl. CPC C12Q 1/6886

(57) ABSTRACT

Methods for diagnosing cancer, its subtypes, molecular features, and likelihood of response to therapy, as well as other diseases, based on microbial presence or abundance in tissues, including blood-derived tissues, of the host subject. Methods of treatment of the identified cancer in subjects are also provided.

### CROSS REFERENCE TO RELATED APPLICATIONS

This application claims the priority benefit of U.S. Provisional Application No. 62/754,696, filed Nov. 2, 2018, which application is incorporated herein by reference.

### TECHNICAL FIELD

The invention relates to the field of methods to accurately diagnose and treat disease using nucleic acids of non-human origin from a human tissue biopsy or blood-derived sample.

### BACKGROUND

Despite a commonly held view that cancer is a ‘disease of the human genome,’ an increasing amount of evidence indicates a key role for microbiota in carcinogenesis, tumor progression, and response to therapy. In fact, as much as 20% of the global cancer burden has been estimated to be caused by microbial agents. Many researchers believe the potential mechanism is through our resident microbes' influence on the immune system, with their abilities to dial up or dampen down inflammation as well as to manipulate the capabilities and responsiveness of our immune cells.

Based on data from studies using gnotobiotic mouse models colonized with one or more specific bacteria, it appears that microbiota can alter cancer susceptibility and progression by diverse mechanisms, such as modulating inflammation, inducing DNA damage, and producing metabolites involved in oncogenesis or tumor suppression. In addition to carcinogenesis and cancer progression, emerging evidence suggests that microbiota can predict response to cancer treatment or be manipulated for improving cancer treatment, including “traditional” chemotherapies (e.g. gemcitabine) and more “innovative” immunotherapies (e.g. PD-1 blockade). Yet, virtually all of this literature has relied on examining variants of the host gut microbiome and its influence on cancer, and the few examples in the literature that have explored cancer tissue specific microbiota—almost universally in gastrointestinal tract cancers—have merely examined questions of pathogenesis. Conversely, no prior art has described broad relationships between non-gastrointestinal microbiota and pan-cancer diagnostics, including from blood-derived samples; similarly, no prior art has described how cancer tissue resident microbiota can predict or impact patient responsiveness to cancer treatment, notably including immunotherapy response. The closest related prior art known to the inventor in this area—US20180291463A1, WO2018200813A1, and WO2018031545A1 (all attributed to Robertson et al.)—relies on a microarray-based technology for detecting pre-selected (“biased”) populations of microbes in tumor tissue samples (NOT blood or other bodily fluids); moreover, this prior art has only covered three cancer types (breast cancer, ovarian cancer, and oral squamous cell carcinoma) rather than taking a pan-cancer approach.

The prior art for this invention builds upon the core concepts of cancer diagnosis using nucleic acids of human origin, either in solid tissue biopsies or liquid (i.e. blood-based) biopsies. It also builds upon the concepts of detecting circulating tumor DNA (ctDNA) to diagnose the presence of a tumor (e.g. PMID: 24553385) and recently described microbial cell-free DNA to detect infectious disease agents in a patient suspected of sepsis (PMID: 30742071). Notably, these host-based ctDNA assays almost always cannot diagnose the kind of cancer since the majority of genomic alterations in cancer are shared between cancer types. From a biological perspective, it has been well known for several years that isolating (via microbial blood culture) certain kinds of bacteria from the blood is highly suggestive of underlying colorectal cancer (e.g. Streptococcus bovis; PMID: 21247505), and a recent study on >13,000 patients demonstrated widespread, transient bacteremias, as detected by traditional blood culture, in those who ended up having colorectal cancer (PMID: 29729257). For blood-based diagnostics, this invention extends the notion of cancer-specific bacteremias to include many more tumor types; it further does not rely on traditional blood culture methods nor does it necessarily require pre-selecting the microbial population of interest and exploits this idea to create a broad diagnostic assay. The invention additionally extends tumor tissue-based diagnostics to discriminate between several dozens of cancer types (i.e. “pan-cancer” diagnostics), their subtypes, their molecular features (e.g. mutations), and their predicted response to therapy, including immunotherapy. Moreover, this invention extends the diagnostic information to select or create new treatments based on intra-tumoral microbial features.

Other prior art that is relevant to this field is as follows: U.S. Publication No. 2018/0223338 describes using the solid tissue microbiome or salvia microbiome in identifying and diagnosing head and neck cancer; and U.S. Publication No. 2018/0258495A1 describes using the solid tissue microbiome or fecal microbiome to detect colon cancer, some kinds of mutations associated with colon cancer, and a kit to collect and amplify the corresponding microbes.

### SUMMARY OF THE INVENTION

The disclosure of the present invention provides a method to accurately diagnose cancer and other diseases, its subtypes, and its likelihood to response to certain therapies solely using nucleic acids of non-human origin from a human tissue biopsy or blood-derived sample.

In embodiments, the invention provides a method for broadly creating patterns of microbial presence or abundance (‘signatures’) that are associated with the presence and/or type of cancer using blood-derived tissues. These ‘signatures’ can then be deployed to diagnose the presence, kind, and/or subtype of cancer in a human.

In embodiments, the invention provides a method for broadly creating patterns of microbial presence or abundance that are associated with the presence and/or type of cancer using primary tumor tissues. These ‘signatures’ can then be deployed to diagnose the presence, kind, and/or subtype of cancer in a human.

In embodiments, the invention provides a method of broadly diagnosing disease in a mammalian subject comprising: detecting microbial presence or abundance in a tissue sample from the subject; determining that the detected microbial presence or abundance is different than microbial presence or abundance in a normal tissue sample, and correlating the detected microbial presence or abundance with a known microbial presence or abundance for a disease, thereby diagnosing the disease.

In embodiments, the invention provides a method of broadly diagnosing the type of disease in a mammalian subject comprising: detecting microbial presence or abundance in a tumor tissue sample from the subject; determining that the detected microbial presence or abundance is similar or different to the microbial presence or abundance in a population of previously studied tumors, and correlating the detected microbial presence or abundance with the most similar tumor type, thereby diagnosing the kind of disease.

In embodiments, the invention provides a method of diagnosing the type of disease in a mammalian subject comprising: detecting microbial presence or abundance in a blood-derived tissue sample from the subject; determining that the detected microbial presence or abundance is similar or different to the microbial presence or abundance in a population of cancer and/or healthy patients with previously studied blood-derived tissue samples, and correlating the detected microbial presence or abundance with the most similar blood-derived tissue samples in this cohort, thereby diagnosing the disease and/or kind of disease.

In embodiments, the invention provides a method of diagnosing the bodily location of disease, wherein the disease is cancer, wherein the location of origin is the bone (acute myelogenous leukemia, sarcoma), the adrenal glands, the bladder, the brain, the breast, the cervix, the gallbladder, the colon, the esophagus, the neck (head and neck squamous cell carcinoma), the kidney, the liver, the lung, the lymph nodes (diffuse large B-cell lymphoma), the skin, the ovary, the prostate, the rectum, the stomach, the thyroid, and the uterus, and wherein the subject is human.

In embodiments, the invention provides a method of diagnosing disease, wherein the disease is cancer, wherein the cancer is leukemia (acute myelogenous), adrenocortical cancer, bladder cancer, brain cancer (lower grade glioma; glioblastoma), breast cancer, cervical cancer, cholangiocarcinoma, colon cancer, esophageal cancer, head and neck cancer, kidney cancer (chromophobe; renal clear cell carcinoma; papillary cell carcinoma), liver cancer, lung cancer (adenocarcinoma; squamous cell carcinoma), lymphoid neoplasm diffuse large B-cell lymphoma, melanoma (skin cutaneous melanoma, uveal melanoma), ovarian cancer, prostate cancer, rectum cancer, sarcoma, stomach cancer, thyroid cancer (thyroid carcinoma, thymoma), and uterine cancer, and wherein the subject is human.

In embodiments, the invention provides a method of diagnosing disease, further comprising diagnosis of the stage of the disease, wherein the disease is cancer.

In embodiments, the invention provides a method of diagnosing disease when the disease is at low pathologic stage, wherein the disease is cancer, wherein the pathologic stage is stage I or stage II.

In embodiments, the invention provides a method of predicting the molecular features of the mammalian disease using non-mammalian features, wherein the mammalian disease is cancer, wherein the molecular features are mutation statuses.

In embodiments, the invention provides a method of predicting which subjects will respond or will not respond to a particular treatment for disease, wherein the disease is cancer, wherein the subject is human, wherein the treatment is immunotherapy, wherein the immunotherapy is a PD-1 blockade (e.g. nivolumab, pembrolizumab).

In embodiments, the invention provides a method of diagnosing disease, further comprising treating the disease in the subject based on the identified non-mammalian features of the disease, wherein the disease is cancer, wherein the non-mammalian features are microbial, wherein the subject is human.

In embodiments, the invention provides a method of diagnosing disease, further comprising designing a new treatment to treat the mammalian disease in the subject based on its non-mammalian features, wherein the disease is cancer, wherein the non-mammalian features are microbial, wherein the subject is human.

In embodiments, new treatments may be designed to target and exploit the non-mammalian features identified in the mammalian disease using one or more of the following modalities: small molecules, biologics, engineered host-derived cell types, probiotics, engineered bacteria, natural-but-selective viruses, engineered viruses, and bacteriophages.

In embodiments, the invention provides a method of diagnosing disease, further comprising longitudinal monitoring of its non-mammalian features to indicate response to treating the disease, wherein the disease is cancer, wherein the non-mammalian features are microbial, wherein the subject is human.

In embodiments, the invention provides a kit to measure the microbial presence or abundance in the specified tissue samples, thereby permitting diagnosis of the disease.

In embodiments, the invention utilizes a diagnostic model based on a machine learning architecture.

In embodiments, the invention utilizes a diagnostic model based on a regularized machine learning architecture.

In embodiments, the invention utilizes a diagnostic model based on an ensemble of machine learning architectures.

In embodiments, the invention identifies and selectively removes certain non-mammalian features as contaminants termed noise, while selectively retaining other non-mammalian features as non-contaminants termed signal, wherein non-mammalian features are microbial.

In embodiments, the invention provides a method of diagnosing disease wherein the microbes are of viral, bacterial, archaeal, and/or fungal origin.

In embodiments, the invention provides a method of diagnosing disease wherein microbial presence or abundance information is combined with additional information about the host (subject) and/or the host's (subject's) cancer to create a diagnostic model that has greater predictive performance than only having microbial presence or abundance information alone.

In embodiments, the diagnostic model utilizes information in combination with microbial presence or abundance information from one or more of the following sources: cell-free tumor DNA, cell-free tumor RNA, exosomal-derived tumor DNA, exosomal-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, and/or methylation patterns of circulating tumor cell derived RNA.

In embodiments, microbial presence or abundance is detected by nucleic acid detection of one or more of the following methods: targeted microbial sequencing (e.g. 16S rRNA sequencing, 18S rRNA ITS sequencing), ecological shotgun sequencing, quantitative polymerase chain reaction (qPCR), immunohistochemistry (IHC), in situ hybridization (ISH), flow cytometry, host whole genome sequencing, host transcriptomic sequencing, cancer whole genome sequencing, and cancer transcriptomic sequencing.

In embodiments, the geospatial distribution of microbial presence or absence is measured in the cancer tissue of the host by one or more of the following methods: multisampling of the tumor tissue and/or its microenvironment, IHC, ISH, digital spatial genomics, digital spatial transcriptomics.

In embodiments, the microbial nucleic acids are detected simultaneously with nucleic acids from the host and subsequently distinguished.

In embodiments, the host nucleic acids are selectively depleted and the microbial nucleic acids are selectively retained prior to measurement (e.g. sequencing) of a combined nucleic acid pool.

In embodiments, the invention provides that the tissue is blood, a constituent of blood (e.g. plasma), or a tissue biopsy, wherein the tissue biopsy may be malignant or non-malignant.

In embodiments, the microbial presence or abundance of the cancer is determined by measuring microbial presence or abundance in other locations of the host.

### BRIEF DESCRIPTION OF THE DRAWINGS

FIGS. 1A-1D: FIG. 1A (left) shows the total percentage of sequencing reads identified as “microbial” by the bioinformatic microbial detection pipeline across 33 cancer types and over 10,000 patients in The Cancer Genome Atlas (TCGA), as well as the percentage of microbial reads retained when summarizing to the genus taxonomy level (right). FIGS. 1B-1C show a principal component analysis (PCA) on normalized (i.e. approximately normal in its distribution) but not batch corrected microbial abundances (1B), as well as normalized and batch corrected microbial abundances (1C). The legend shows that the data were derived from eight sequencing centers in total. FIG. 1D shows the results of a principal variance component analysis (PVCA) before and after batch correction to estimate the amount of microbial variance (“signal”) attributed across each major metadata variable in the dataset. Fold-increases and fold-decreases are shown above the major metadata variables that changed during the batch correction process.

FIGS. 2A-2F: In FIG. 2A, patients that were clinically evaluated for HPV-infected cervical squamous cell carcinoma and endocervical adenocarcinoma were examined for differential abundance of the Alphapapillomavirus genus in their tumors and matched blood samples. Primary tumor samples are compared as a positive control and blood derived normal samples are compared as a negative control. In FIG. 2B, patients that were clinically evaluated for HPV-infected head and neck squamous cell carcinoma (TCGA-HNSCC; primary tumor samples) were compared for differential abundance of the Alphapapillomavirus genus using both in situ hybridization (ISH) and immunohistochemistry (IHC) assays (p16). In FIG. 2C, patients with stomach adenocarcinoma that were assigned integrative molecular subtypes by The Cancer Genome Atlas Research Network and those in the Epstein-Barr virus (EBV) subtype were examined for selective overabundance of the EBV genus (i.e. Lymphocrytovirus). Blood derived normal and solid tissue normal samples are shown as negative controls. Other molecular subtypes of STAD: CIN=chromosomal instability; GS=genome stable; MSI=microsatellite unstable. In FIG. 2D, patients with clinically adjudicated risk factors for liver hepatocellular cancer were plotted against the normalized abundance of the Orthohepadnavirus genus to examine selective overabundance of the Orthohepadnavirus genus in patients with a history of hepatitis B infection. “EtOH” denotes heavy alcohol consumption as a prior risk factor while “Hep C” denotes prior hepatitis C infection. Blood derived normal samples are shown as negative controls; solid tissue normals reveal high viral loads of hepatitis B. In FIG. 2E, common gastrointestinal cancers were evaluated for differential abundances of the Fusobacterium genus, as associated in the literature. Blood derived normals and solid tissue normals are shown for comparative negative controls. In FIG. 2F, abundances of the Fusobacterium genus were examined between gastrointestinal tract (GI-tract) cancers and non-GI-tract cancers. The following cancers were included in the GI-tract group: colon adenocarcinoma, rectum adenocarcinoma, cholangiocarcinoma, liver hepatocellular carcinoma, pancreatic adenocarcinoma, head and neck squamous cell carcinoma, esophageal carcinoma, and stomach adenocarcinoma. The remaining cancer types in Table 1 were placed in the non-GI-tract cancers with the exception of acute myeloid leukemia, which was excluded from this analysis. Fusobacterium abundance from adjacent non-malignant tissue is included from both groups as a negative control. For all figures: The y-axis shows normalized microbial abundances on a log₂ scale; significance testing was performed using a two-sided Mann-Whitney test for all comparisons; symbols are as follows: **** for p-values<=0.0001, *** for p-values<=0.001, ** for p-values<=0.01, * for p-values<=0.05, and “ns” for not significant.

FIG. 3: The distribution of Alphapapillomavirus genus abundance across 32 cancer types and 3 sample types (solid tissue normal, blood derived normal, and primary tumor tissues). For cancer types that had patients who were clinically adjudicated for HPV infection, the cancer types are split into groups that either tested “Positive” or “Negative” for HPV infection. The dotted lines are the average abundance values for all patients that tested “Negative” within each sample type.

FIGS. 4A-4F: Whole transcriptome data (RNA-Seq) collected by Hugo et al. (2016; Science; PMID: 26997480) on patients prior to receiving anti-PD-1 immunotherapy (pembrolizumab or nivolumab) were explored for microbial RNA reads. FIG. 4A shows the principal co-ordinate analysis for patients with complete response (CR) versus those with progressive disease (PD). “Adonis” denotes a PERMANOVA test for significant separation between the two centroids of the groups. FIG. 4B shows the distances of each patient to his or her respective centroid (i.e. CR or PD), which is a measure of beta-diversity, namely that patients with CR have distinguishably lower beta dispersion than those with PD. “Betadisper Perm Test” denotes a permutation test to discern if the beta dispersion is significantly different between the groups. FIG. 4C shows the principal co-ordinate analysis for patients with complete response (CR) versus those with partial response (PR). “Adonis” denotes a PERMANOVA test for significant separation between the two centroids of the groups. FIG. 4D shows the distances of each patient to his or her respective centroid (i.e. CR or PR), which is a measure of beta-diversity, namely that patients with CR have distinguishably lower beta dispersion than those with PR. “Betadisper Perm Test” denotes a permutation test to discern if the beta dispersion is significantly different between the groups. FIG. 4E shows the ROC and PR curves (i.e. machine learning model performance) for predicting microsatellite instability in TCGA colon adenocarcinoma samples solely using microbial DNA or RNA abundances. These performances are based on a randomly selected, 30% holdout test set after the model was trained on 70% of the data and internally parameterized using k-fold cross validation of the training data. FIG. 4F shows the ROC and PR curves for predicting which TCGA breast cancer samples are triple negative or not. These performances are based on a randomly selected, 30% holdout test set after the model was trained on 70% of the data and internally parameterized using k-fold cross validation of the training data.

FIGS. 5A-5F: ROC and PR curves for the following cancer types: Adrenocortical carcinoma, bladder urothelial carcinoma. Exemplar arrows are given in the first ROC and PR plots and point to respective extrema locations on the plots for a given probability cutoff threshold of 1.0 or 0.0; the rest of the probability cutoff threshold spectrum, as well as their respective ROC or PR points, span proportionately between the two points on the plots that are indicated by the arrows. Abbreviations are as follows: “PT” denotes “Primary Tumor”, “BDN” denotes “Blood Derived Normal”, and “STN” denotes “Solid Tissue Normal”. For “PT” and “BDN” labeled figures, predictions were done in a one-cancer-type-versus-all-others fashion; for “PT vs STN” labeled figures, predictions were done to discriminate primary tumor tissue versus adjacent solid tissue normal within a given cancer type. All prediction performances were generated on a randomly selected, 30% holdout test set after the respective model was trained on the remaining 70% of the data for a given comparison; during model training, k-fold cross validation was employed to tune the model parameters. Additionally, in cases of class imbalance, the minority class was up-sampled to promote model generalization.

FIGS. 6A-6F: ROC and PR curves for the following cancer types: Bladder urothelial carcinoma, brain lower grade glioma. Abbreviations are given in the caption for FIGS. 5A-5F. Model performances were generated the same way as described in the caption for FIGS. 5A-5F.

FIGS. 7A-7F: ROC and PR curves for the following cancer types: Breast invasive carcinoma. Abbreviations are given in the caption for FIGS. 5A-5F. Model performances were generated the same way as described in the caption for FIGS. 5A-5F.

FIGS. 8A-8F: ROC and PR curves for the following cancer types: Cervical squamous cell carcinoma and endocervical adenocarcinoma, cholangiocarcinoma. Abbreviations are given in the caption for FIGS. 5A-5F. Model performances were generated the same way as described in the caption for FIGS. 5A-5F.

FIGS. 9A-9F: ROC and PR curves for the following cancer types: Colon adenocarcinoma. Abbreviations are given in the caption for FIGS. 5A-5F. Model performances were generated the same way as described in the caption for FIGS. 5A-5F.

FIGS. 10A-10F: ROC and PR curves for the following cancer types: Esophageal carcinoma. Abbreviations are given in the caption for FIGS. 5A-5F. Model performances were generated the same way as described in the caption for FIGS. 5A-5F.

FIGS. 11A-11F: ROC and PR curves for the following cancer types: Glioblastoma multiforme, head and neck squamous cell carcinoma. Abbreviations are given in the caption for FIGS. 5A-5F. Model performances were generated the same way as described in the caption for FIGS. 5A-5F.

FIGS. 12A-12F: ROC and PR curves for the following cancer types: Head and neck squamous cell carcinoma, kidney chromophobe. Abbreviations are given in the caption for FIGS. 5A-5F. Model performances were generated the same way as described in the caption for FIGS. 5A-5F.

FIGS. 13A-13F: ROC and PR curves for the following cancer types: Kidney chromophobe, kidney renal clear cell carcinoma. Abbreviations are given in the caption for FIGS. 5A-5F. Model performances were generated the same way as described in the caption for FIGS. 5A-5F.

FIGS. 14A-14F: ROC and PR curves for the following cancer types: Kidney renal papillary cell carcinoma. Abbreviations are given in the caption for FIGS. 5A-5F. Model performances were generated the same way as described in the caption for FIGS. 5A-5F.

FIGS. 15A-15F: ROC and PR curves for the following cancer types: Liver hepatocellular carcinoma. Abbreviations are given in the caption for FIGS. 5A-5F. Model performances were generated the same way as described in the caption for FIGS. 5A-5F.

FIGS. 16A-16F: ROC and PR curves for the following cancer types: Lung adenocarcinoma. Abbreviations are given in the caption for FIGS. 5A-5F. Model performances were generated the same way as described in the caption for FIGS. 5A-5F.

FIGS. 17A-17F: ROC and PR curves for the following cancer types: Lung squamous cell carcinoma, lymphoid neoplasm diffuse large B-cell lymphoma. Abbreviations are given in the caption for FIGS. 5A-5F. Model performances were generated the same way as described in the caption for FIGS. 5A-5F.

FIGS. 18A-18F: ROC and PR curves for the following cancer types: Mesothelioma, ovarian serous cystadenocarcinoma. Abbreviations are given in the caption for FIGS. 5A-5F. Model performances were generated the same way as described in the caption for FIGS. 5A-5F.

FIGS. 19A-19F: ROC and PR curves for the following cancer types: Pancreatic adenocarcinoma, pheochromocytoma and paraganglioma, prostate adenocarcinoma. Abbreviations are given in the caption for FIGS. 5A-5F. Model performances were generated the same way as described in the caption for FIGS. 5A-5F.

FIGS. 20A-20F: ROC and PR curves for the following cancer types: Prostate adenocarcinoma, rectum adenocarcinoma. Abbreviations are given in the caption for FIGS. 5A-5F. Model performances were generated the same way as described in the caption for FIGS. 5A-5F.

FIGS. 21A-21F: ROC and PR curves for the following cancer types: Rectum adenocarcinoma, sarcoma. Abbreviations are given in the caption for FIGS. 5A-5F. Model performances were generated the same way as described in the caption for FIGS. 5A-5F.

FIGS. 22A-22F: ROC and PR curves for the following cancer types: Skin cutaneous melanoma, stomach adenocarcinoma. Abbreviations are given in the caption for FIGS. 5A-5F. Model performances were generated the same way as described in the caption for FIGS. 5A-5F.

FIGS. 23A-23F: ROC and PR curves for the following cancer types: Stomach adenocarcinoma, testicular germ cell tumors. Abbreviations are given in the caption for FIGS. 5A-5F. Model performances were generated the same way as described in the caption for FIGS. 5A-5F.

FIGS. 24A-24F: ROC and PR curves for the following cancer types: Thymoma, thyroid carcinoma. Abbreviations are given in the caption for FIGS. 5A-5F. Model performances were generated the same way as described in the caption for FIGS. 5A-5F.

FIGS. 25A-25F: ROC and PR curves for the following cancer types: Thyroid carcinoma, uterine carcinosarcoma, uterine corpus endometrial carcinoma. Abbreviations are given in the caption for FIGS. 5A-5F. Model performances were generated the same way as described in the caption for FIGS. 5A-5F.

FIGS. 26A-26F: ROC and PR curves for the following cancer types: Uterine corpus endometrial carcinoma, uveal melanoma. Abbreviations are given in the caption for FIGS. 5A-5F. Model performances were generated the same way as described in the caption for FIGS. 5A-5F.

FIGS. 27A-27B: ROC and PR curves for the following cancer types: Uveal melanoma. Abbreviations are given in the caption for FIGS. 5A-5F. Model performances were generated the same way as described in the caption for FIGS. 5A-5F.

FIG. 28: FIG. 28A shows one embodiment of a decontamination pipeline, which strives to identify and subsequently remove contaminating microbes (“noise”) while retaining non-contaminating microbes (“signal”) from primary surgical resection of the tissue through nucleic acid sequencing and data analysis. FIGS. 28B and 28C show the comparative model performances as areas under ROC and PR curves, respectively, on models built on full (“non-decontaminated”) data and on decontaminated data. A linear regression with a gray standard error bar ribbon is shown of the data points; a diagonal line is shown to denote what perfect (1:1) correspondence would be between the two sets of model performances. In this particular embodiment, microbial taxonomies that were suspected to be contaminants by the decontamination pipeline (cf. FIG. 28A) were entirely removed prior to model building and testing. As before, the models were built and tested as described in FIGS. 5A-5F, namely that the predictions were one-cancer-type-versus-all-others using either “Primary Tumor” or “Blood Derived Normal” tissues. Model performances were generated on randomly selected, 30% holdout test sets after training the model on the remaining 70% of the data with internal k-fold cross validation for model parameterization.

FIGS. 29A-29I: FIG. 29A shows one embodiment of validating the model performances observed in FIGS. 5A-27B. Specifically, before normalization and batch correction, the raw microbial count data were split in half in a stratified manner Each raw data half was then processed through the normalization and batch correction pipelines prior to machine learning model building. In this case, the model learning model that was built on the first half was tested on the second half, and vice versa. The resultant model performances were compared to building a model on 50% of the full, non-subsetted, normalized, batch corrected data and then subsequently testing on the remaining 50% of the full, non-subsetted, normalized, batch corrected data. Area under the curve values for ROC and PR curves are shown and labeled in the heatmap with each row being (and labeled as) a distinct TCGA cancer type (see Table 1 for abbreviations). FIGS. 29B and 29C show comparative model performance (ROC and PR curve areas) between models that were built to discriminate between one cancer type versus all others using both DNA and RNA (“full data”) or just RNA. All microbial DNA and/or RNA came from primary tumors in TCGA and each data point is respectively labeled with a TCGA cancer type. Model performance was generated by applying the trained model on a randomly selected, 30% holdout test set. FIGS. 29D and 29E show comparative model performance (ROC and PR curve areas) between models that were built to discriminate between one cancer type versus all others using both DNA and RNA (“full data”) or just DNA. All microbial RNA and/or DNA came from primary tumors in TCGA and each data point is respectively labeled with a TCGA cancer type. Model performance was generated by applying the trained model on a randomly selected, 30% holdout test set. FIGS. 29F and 29G show comparative model performance (ROC and PR curve areas) between models that were built to discriminate between one cancer type versus all others using sequencing data from all eight TCGA sequencing centers (“full data”) or just from the University of North Carolina (UNC). Notably, all sequencing data from UNC was only RNA (RNA-Seq), so this comparison eliminates possible variation due to incorporating multiple sequencing centers and experimental types. All microbial DNA and/or RNA came from primary tumors in TCGA and each data point is respectively labeled with a TCGA cancer type. Model performance was generated by applying the trained model on a randomly selected, 30% holdout test set. FIGS. 29H and 29I show comparative model performance (ROC and PR curve areas) between models that were built to discriminate between one cancer type versus all others using sequencing data from all eight TCGA sequencing centers (“full data”) or just from the Harvard Medical School (HMS). Notably, all sequencing data from HMS was only DNA (Whole Genome Sequencing, WGS), so this comparison eliminates possible variation due to incorporating multiple sequencing centers and experimental types. All microbial RNA and/or DNA came from primary tumors in TCGA and each data point is respectively labeled with a TCGA cancer type. Model performance was generated by applying the trained model on a randomly selected, 30% holdout test set.

FIGS. 30A-30J: The mutation status of the top five most frequent mutations in TCGA (TP53, PTEN, PIK3CA, ARID1A, APC) are predicted solely by intratumoral microbial DNA and RNA abundances. The areas under the ROC and PR curves are shown on each respective plot.

FIG. 31: For benchmarking purposes, all patients with stage I and stage II cancers in TCGA were explored for discriminative performance between cancer types solely using microbial DNA identified in their matched blood samples. Models were built and tested as previously described: 70% of the data (randomly selected) were used for training discriminative models with internal k-fold cross validation for model tuning and final performance values were generated on the remaining, held-out 30% of the data; predictions were one-cancer-type-versus-all-others solely using microbial DNA. Additionally, model performance was compared across three levels of decontamination stringency, which resulted in models being built on four distinct datasets with varying proportions of original microbes being removed; for example, in the “Most Stringent Filtering” embodiment, over 90% of the original reads and taxa were discarded. One skilled in the art will recognize that there are many possible variations of decontamination stringency that are employable here and that model performance may be improved or worsened by shifting that stringency level higher or lower.

FIGS. 32A-32C: For a conservative, comparative analysis against existing cell-free tumor DNA (ctDNA) assays, all TCGA patients containing at least one mutation in their tumor that was examined by two commercial ctDNA assays (GUARDANT360, FOUNDATIONONE Liquid) were removed. The remaining patients, whose cancers thus cannot be detected under any circumstances using these two commercial ctDNA assays, had microbial DNA extracted from their matched blood samples in TCGA. Using this microbial DNA, machine learning models were subsequently trained and tested to predict one cancer type versus all others; as before, performance was generated based on applying the model to a randomly selected, 30% holdout test set. The resultant model performances for patients without any detectable genomic alterations on the GUARDANT360 ctDNA panel are shown in FIG. 32A; similarly, model performances for patients without any detectable genomic alterations on the FOUNDATIONONE Liquid ctDNA panel are shown in FIG. 32B. The exact list of genomic alterations examined by these commercial ctDNA assay panels are listed in FIG. 32C

FIGS. 33A-33B: A website was developed to host and display the microbial presence and abundance information across dozens of cancer types in TCGA (FIG. 33A), as well as to show the discriminatory performance of models in one-cancer-type-versus-all-others and tumor-vs-normal comparisons and their ranked microbial features (FIG. 33B).

### DETAILED DESCRIPTION

The invention provides, in embodiments, a method to accurately diagnose human cancer, its subtypes, and its likelihood of therapy response using nucleic acids of non-human origin from a human tissue biopsy, malignant or non-malignant, or a blood-derived sample. It does this by identifying specific patterns of microbial nucleic acids and their presence or abundances (‘a signature’) within the sample to assign a certain probability that the sample (1) originated from a tumor rather than a ‘normal’ tissue site (e.g. the sample was a surgically resected solid tissue biopsy); (2) that the individual has cancer (e.g. the sample came from typical blood draw with or without the intention to diagnose cancer); (3) that the individual has a cancer from a particular body site (e.g. the sample came from typical blood draw with or without the intention to diagnose cancer); (4) that the individual has a particular type of cancer (e.g. a patient with suspected cancer has a blood draw taken to quickly diagnose which cancer it may be instead of doing radiation-based imaging studies [e.g. PET-CT] or other costly imaging studies [e.g. MRI]; alternatively, a tissue biopsy of a newly found tumor lesion may be taken and the microbial ‘signature’ may be indicative of what kind of cancer type it is); (5) that a cancer, which may or may not be diagnosed at the time, has a high or low likelihood or responding to a particular cancer therapy (e.g. a tissue biopsy of a suspected tumor lesion is taken, for which a microbial ‘signature’ provides a prediction of whether the patient will respond to therapy or not; alternatively, a blood sample from the same patient may be used, for which a microbial ‘signature’ may predict the immunogenicity of a patient's tumor); (6) that a cancer, which may or may not be diagnosed at the time, is found to harbor microbial features (e.g. microbial antigens) that can be targeted for developing a personalized therapeutic to treat the subject's cancer (e.g. a solid tissue biopsy reveals unique microbial neoantigens in the tumor tissue that can be used to develop a personalized cancer vaccine for the subject). Other uses for such methods are reasonably imaginable and readily implementable to those skilled in the art.

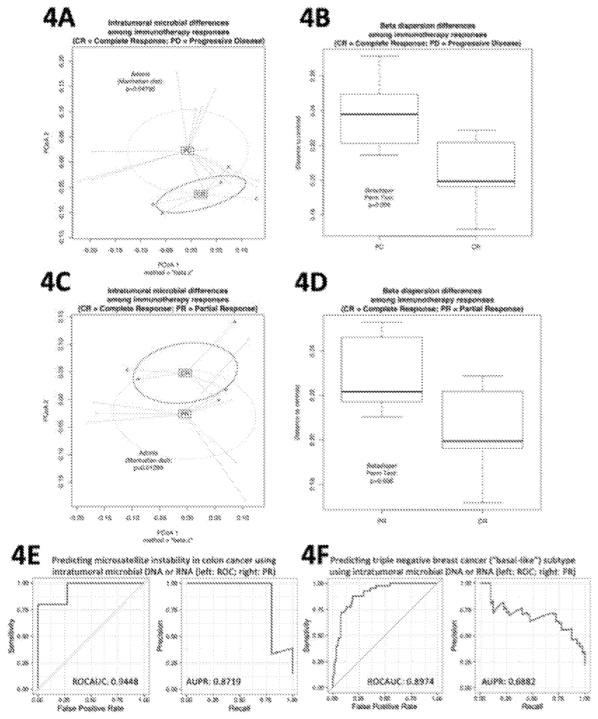

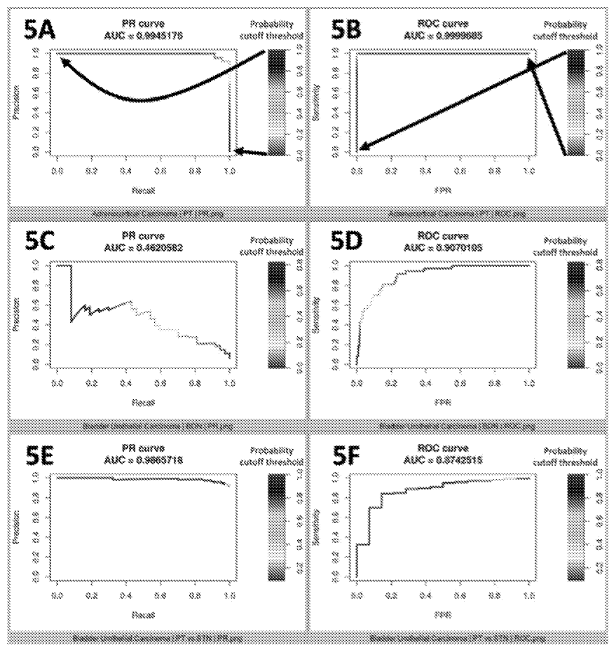

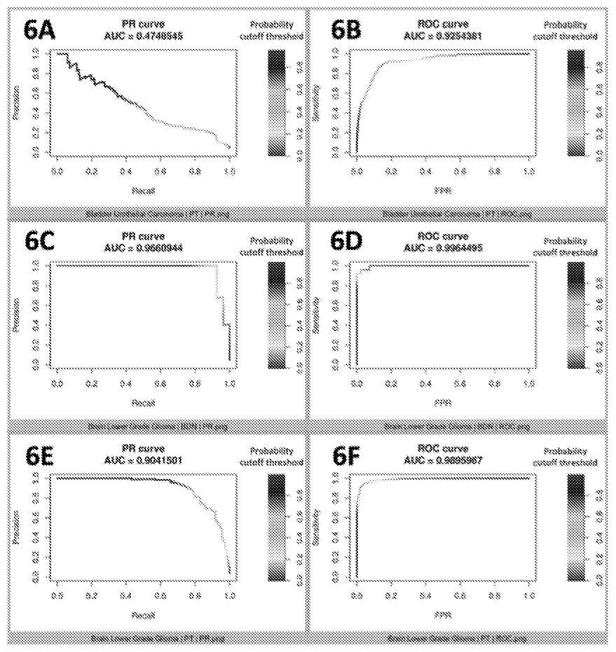

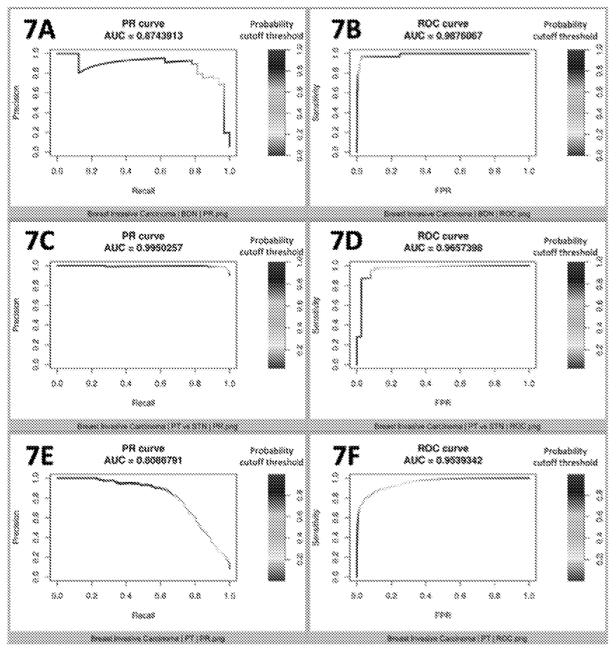

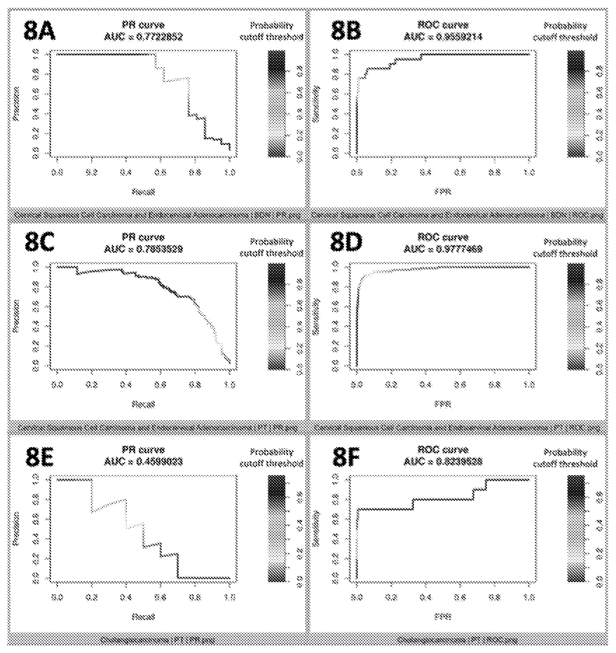

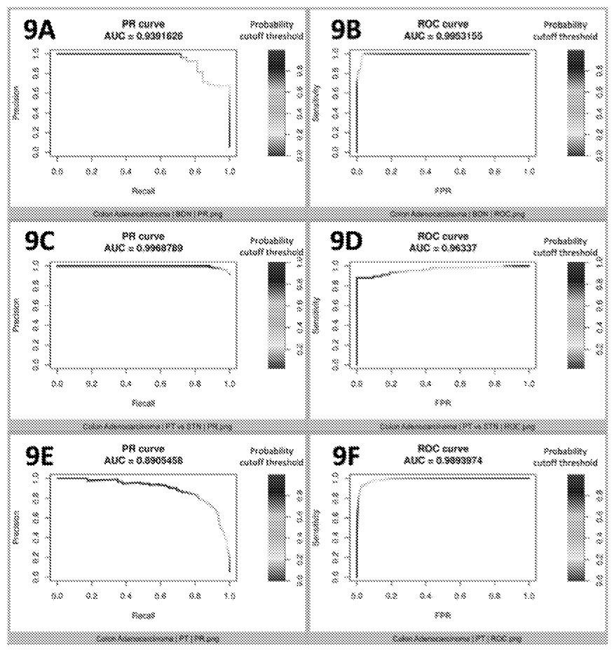

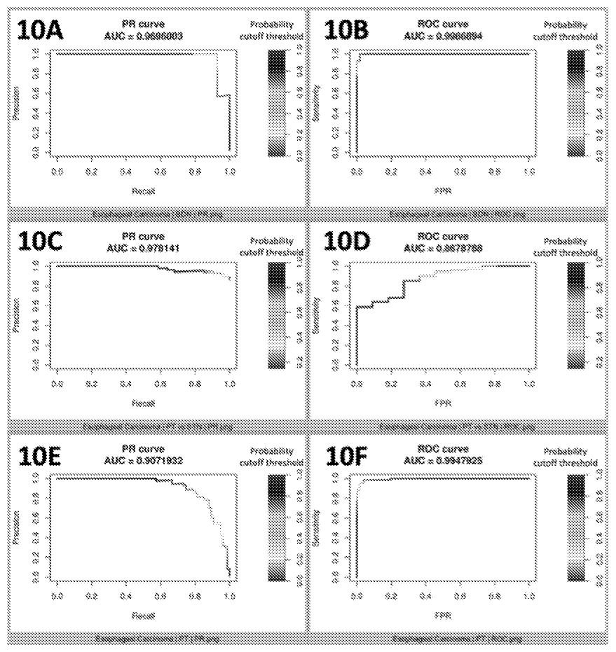

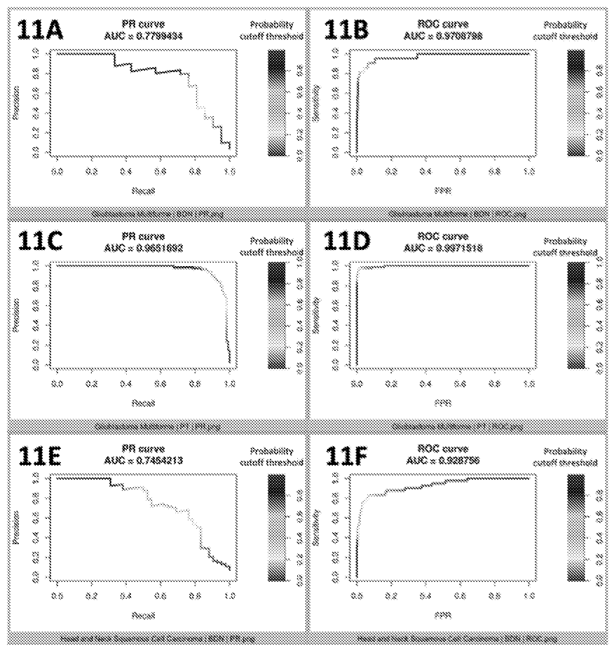

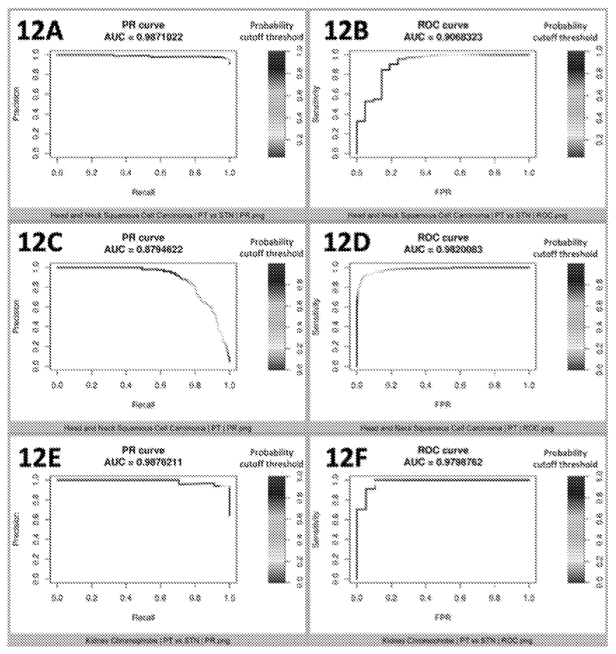

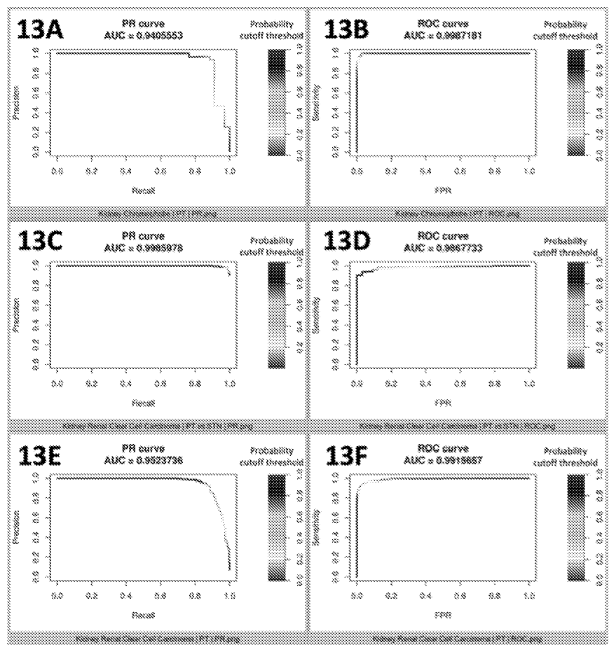

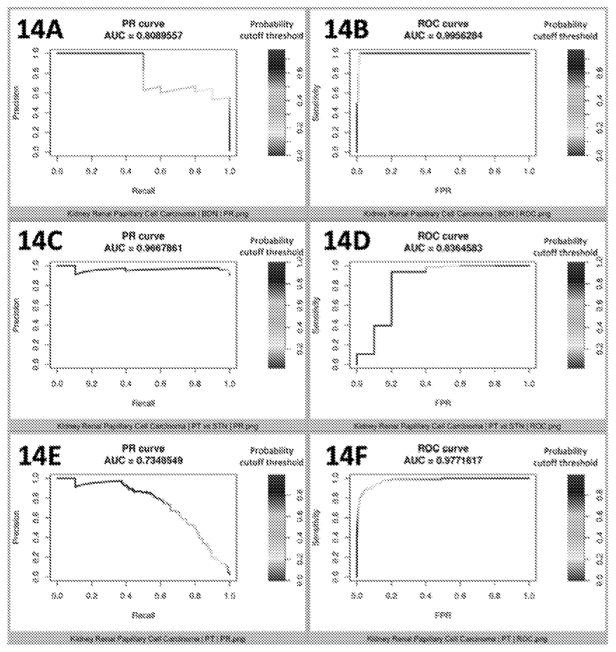

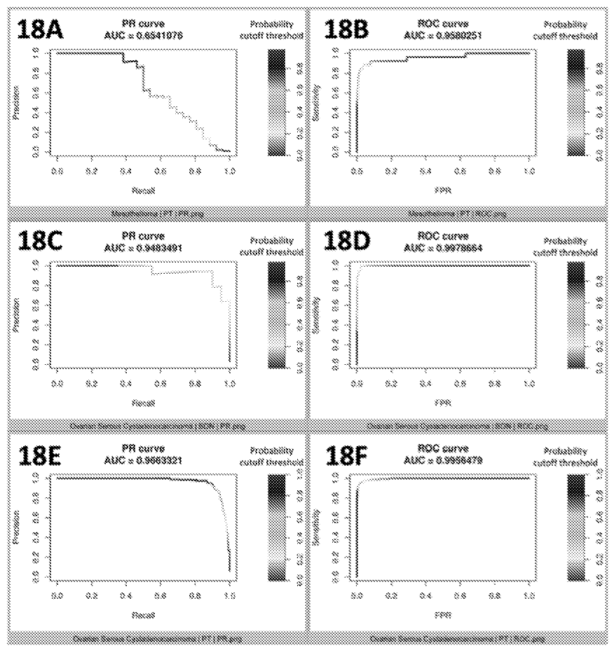

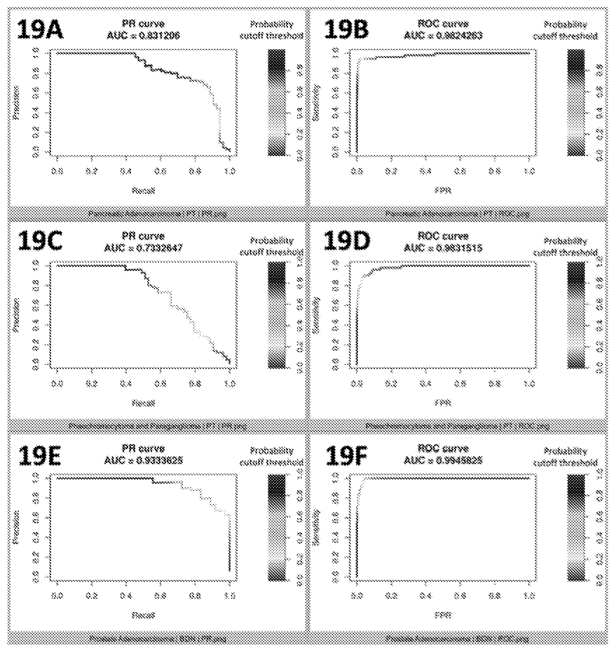

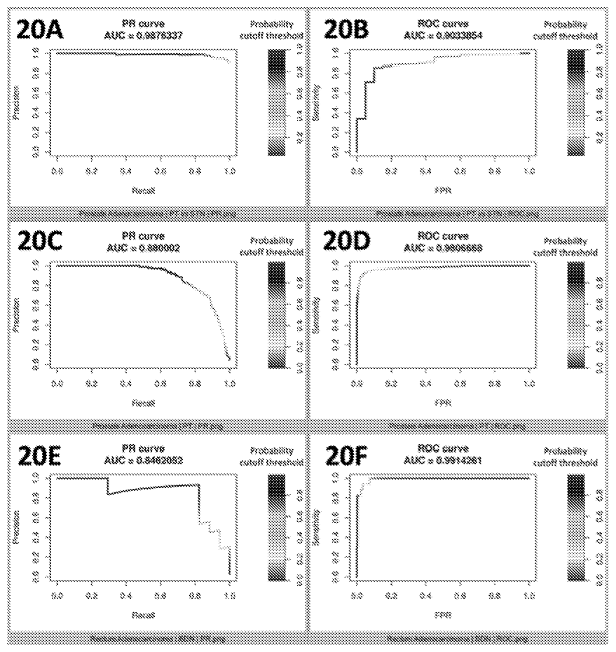

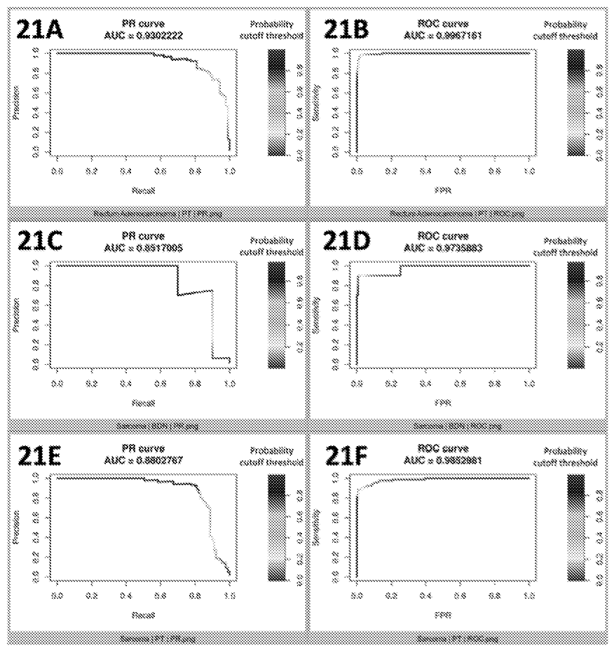

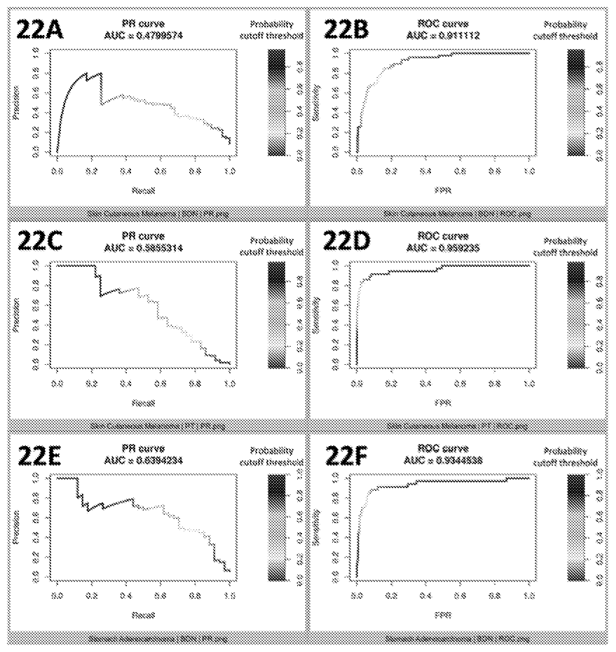

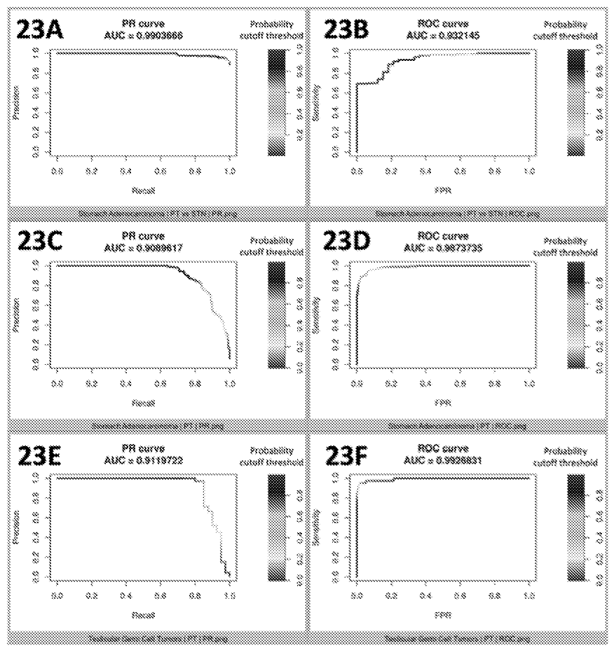

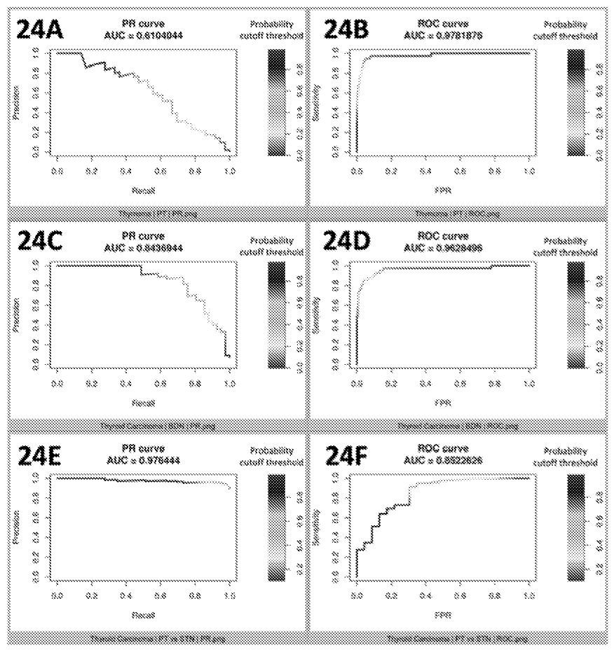

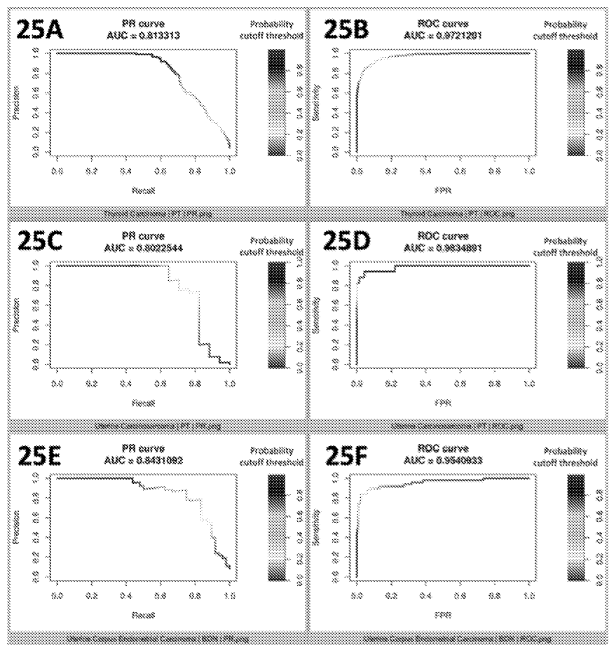

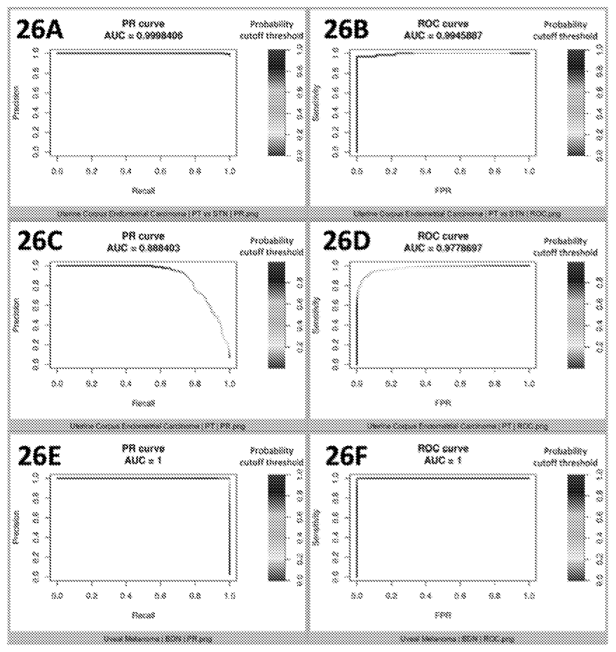

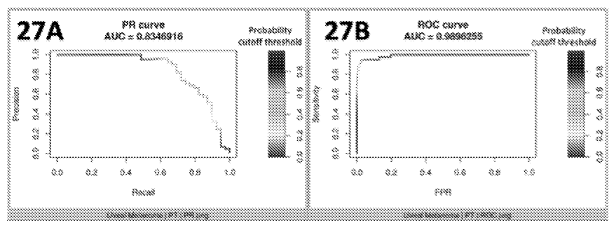

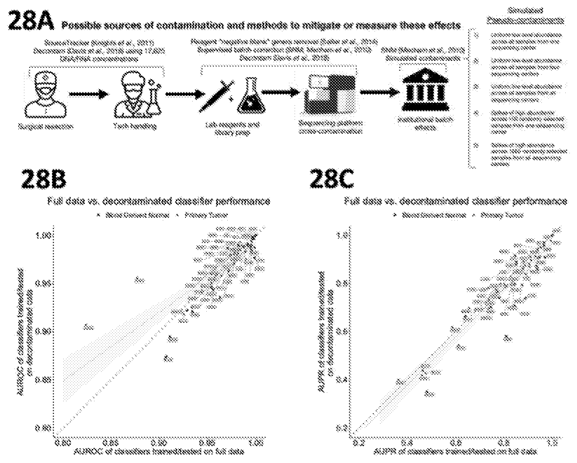

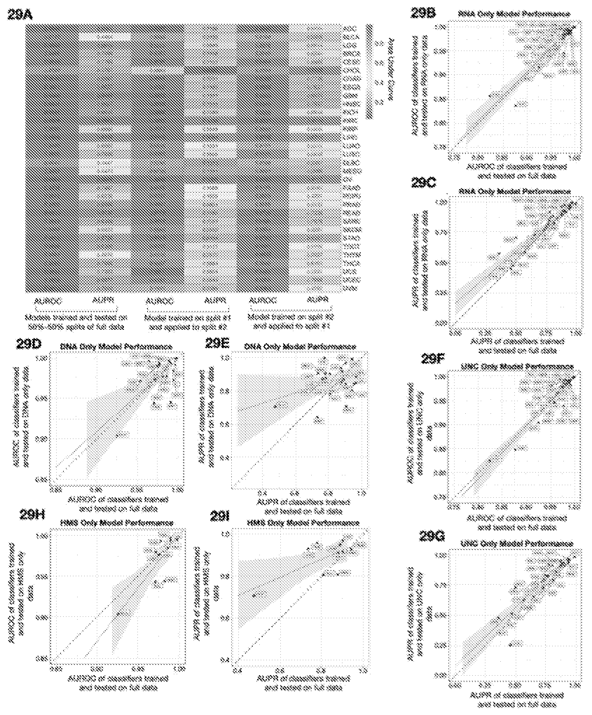

The invention is novel, in part, because it uses nucleic acids of non-human origin to diagnose a condition (i.e. cancer) that has been traditionally thought to be a disease of the human genome. It is better than a typical pathology report because it does not necessarily rely upon observed tissue structure, cellular atypia, or any other subjective measure traditionally used to diagnose cancer. It also has much better sensitivity by focusing solely on microbial sources rather than modified human (i.e. cancerous) sources, which are modified often at extremely low frequencies in a background of ‘normal’ human sources. It can be done using either solid tissue or blood derived samples, the latter of which requires minimal sample preparation and is minimally invasive. It can also predict response to therapies that remain challenging to prognose, including distinguishing ‘complete responders’ to immunotherapy versus subjects who will experience ‘progressive disease’. In certain circumstances, it can further provide information about host molecular aberrations and processes, such as mutation status of a subject's cancer. The blood-based assay additionally does not deal with the same challenges posed by circulating tumor DNA (ctDNA) assays, which can have sensitivity issues due to cell-free DNA (cfDNA) that originates from non-malignant human cells. Moreover, based on data presented in FIGS. 5A-27B, the blood-based microbial assay can distinguish between cancer types, which ctDNA assays most often cannot do, since most common cancer genomic aberrations are shared between cancer types (e.g. TP53 mutations, KRAS mutations). By constraining the size of the signatures, the method of which will be expected by someone knowledgeable in the art (e.g. regularized machine learning), the microbial assays can be made clinically available through the use of e.g. multiplexed qPCR, ISH, or table-top sequencers (e.g. MinION, MiniSeq).

The machine learning models herein containing the microbial signatures can be deployed on real-time sequencing data or retrospective sequencing data. The signatures themselves were developed originally from data that was intended to sequence host nucleic acids but also included, but did not analyze, microbial features (i.e. human whole genome sequencing and RNA-Seq). These include sequencing studies performed on over 17,000 samples, over 10,000 patients, and several dozens of cancer types from patients in geographically diverse regions. However, the input data for these models can also derived from targeted metagenomic studies if so desired (e.g. 16S rRNA sequencing, shotgun sequencing). Moreover, such microbial presence or abundance information may be combined with host nucleic acid information to improve the predictive performance of these models in practice. Reduced to practice, this may or may not include doing the following (i.e. other examples are possible and will be anticipated by those skilled in the art):
Taking a blood sample from a patient during a routine clinic visit;
Removing an aliquot of that blood sample, extracting the nucleic acids within, and amplifying the sequences for specific microbial genes that are indicative of microbial taxonomy (e.g. V4 region of 16S rRNA gene);
Obtaining a digital read-out of the presence and/or abundance of these microbial sequences;
Normalizing the presence and/or abundance data on an adjacent computer or cloud computing infrastructure and feeding it into a previously trained machine learning model;
Reading out a prediction and a certain degree of confidence for how likely this sample (1) is associated with the presence or absence of cancer, (2) is associated with cancer of a particular type or bodily location, or (3) is associated with a high, intermediate, or low likelihood of response to a range of cancer therapies; and
Using that sample's microbial information to continue training the machine learning model if additional information is later inputted by the user.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, 2^{nd} ed. (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction (Mullis et al., eds., 1994); Remington, The Science and Practice of Pharmacy, 20^{th} ed., (Lippincott, Williams & Wilkins 2003), and Remington, The Science and Practice of Pharmacy, 22^{th} ed., (Pharmaceutical Press and Philadelphia College of Pharmacy at University of the Sciences 2012).

### Definitions

To facilitate understanding of the invention, a number of terms and abbreviations as used herein are defined below as follows:

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It is understood that aspects and embodiments of the invention described herein include “consisting” and/or “consisting essentially of” aspects and embodiments.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

As used herein, “patient” or “subject” means a human or mammalian animal subject to be treated.

As used herein the term “pharmaceutical composition” refers to a pharmaceutical acceptable compositions, wherein the composition comprises a pharmaceutically active agent, and in some embodiments further comprises a pharmaceutically acceptable carrier. In some embodiments, the pharmaceutical composition may be a combination of pharmaceutically active agents and carriers.

As used herein the term “pharmaceutically acceptable carrier” refers to an excipient, diluent, preservative, solubilizer, emulsifier, adjuvant, and/or vehicle with which demethylation compound(s), is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

As used herein, “therapeutically effective” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to age-related eye diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses.

As used herein, the terms “treat,” “treatment,” or “treating” embraces at least an amelioration of the symptoms associated with diseases in the patient, where amelioration is used in a broad sense to refer to at least a reduction in the magnitude of a parameter, e.g. a symptom associated with the disease or condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

“Amplification” refers to any known procedure for obtaining multiple copies of a target nucleic acid or its complement, or fragments thereof. The multiple copies may be referred to as amplicons or amplification products. Amplification, in the context of fragments, refers to production of an amplified nucleic acid that contains less than the complete target nucleic acid or its complement, e.g., produced by using an amplification oligonucleotide that hybridizes to, and initiates polymerization from, an internal position of the target nucleic acid. Known amplification methods include, for example, replicase-mediated amplification, polymerase chain reaction (PCR), reverse transcription polymerase chain reaction (RT-PCR), ligase chain reaction (LCR), strand-displacement amplification (SDA), and transcription-mediated or transcription-associated amplification. Amplification is not limited to the strict duplication of the starting molecule. For example, the generation of multiple cDNA molecules from RNA in a sample using reverse transcription (RT)-PCR is a form of amplification. Furthermore, the generation of multiple RNA molecules from a single DNA molecule during the process of transcription is also a form of amplification. During amplification, the amplified products can be labeled using, for example, labeled primers or by incorporating labeled nucleotides.

“Amplicon” or “amplification product” refers to the nucleic acid molecule generated during an amplification procedure that is complementary or homologous to a target nucleic acid or a region thereof. Amplicons can be double stranded or single stranded and can include DNA, RNA or both. Methods for generating amplicons are known to those skilled in the art.

“Codon” refers to a sequence of three nucleotides that together form a unit of genetic code in a nucleic acid.

“Codon of interest” refers to a specific codon in a target nucleic acid that has diagnostic or therapeutic significance (e.g. an allele associated with viral genotype/subtype or drug resistance).

“Complementary” or “complement thereof” means that a contiguous nucleic acid base sequence is capable of hybridizing to another base sequence by standard base pairing (hydrogen bonding) between a series of complementary bases.

Complementary sequences may be completely complementary (i.e. no mismatches in the nucleic acid duplex) at each position in an oligomer sequence relative to its target sequence by using standard base pairing (e.g., G:C, A:T or A:U pairing) or sequences may contain one or more positions that are not complementary by base pairing (e.g., there exists at least one mismatch or unmatched base in the nucleic acid duplex), but such sequences are sufficiently complementary because the entire oligomer sequence is capable of specifically hybridizing with its target sequence in appropriate hybridization conditions (i.e. partially complementary). Contiguous bases in an oligomer are typically at least 80%, preferably at least 90%, and more preferably completely complementary to the intended target sequence.

“Configured to” or “designed to” denotes an actual arrangement of a nucleic acid sequence configuration of a referenced oligonucleotide. For example, a primer that is configured to generate a specified amplicon from a target nucleic acid has a nucleic acid sequence that hybridizes to the target nucleic acid or a region thereof and can be used in an amplification reaction to generate the amplicon. Also as an example, an oligonucleotide that is configured to specifically hybridize to a target nucleic acid or a region thereof has a nucleic acid sequence that specifically hybridizes to the referenced sequence under stringent hybridization conditions.

“Polymerase chain reaction” (PCR) generally refers to a process that uses multiple cycles of nucleic acid denaturation, annealing of primer pairs to opposite strands (forward and reverse), and primer extension to exponentially increase copy numbers of a target nucleic acid sequence. In a variation called RT-PCR, reverse transcriptase (RT) is used to make a complementary DNA (cDNA) from mRNA, and the cDNA is then amplified by PCR to produce multiple copies of DNA. There are many permutations of PCR known to those of ordinary skill in the art.

“Position” refers to a particular amino acid or amino acids in a nucleic acid sequence.

“Primer” refers to an enzymatically extendable oligonucleotide, generally with a defined sequence that is designed to hybridize in an antiparallel manner with a complementary, primer-specific portion of a target nucleic acid. A primer can initiate the polymerization of nucleotides in a template-dependent manner to yield a nucleic acid that is complementary to the target nucleic acid when placed under suitable nucleic acid synthesis conditions (e.g. a primer annealed to a target can be extended in the presence of nucleotides and a DNA/RNA polymerase at a suitable temperature and pH). Suitable reaction conditions and reagents are known to those of ordinary skill in the art. A primer is typically single stranded for maximum efficiency in amplification, but may alternatively be double stranded. If double stranded, the primer is generally first treated to separate its strands before being used to prepare extension products. The primer generally is sufficiently long to prime the synthesis of extension products in the presence of the inducing agent (e.g. polymerase). Specific length and sequence will be dependent on the complexity of the required DNA or RNA targets, as well as on the conditions of primer use such as temperature and ionic strength. Preferably, the primer is about 5-100 nucleotides. Thus, a primer can be, e.g., 5, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 75, 80, 85, 90, 95 or 100 nucleotides in length. A primer does not need to have 100% complementarity with its template for primer elongation to occur; primers with less than 100% complementarity can be sufficient for hybridization and polymerase elongation to occur. A primer can be labeled if desired. The label used on a primer can be any suitable label, and can be detected by, for example, spectroscopic, photochemical, biochemical, immunochemical, chemical, or other detection means. A labeled primer therefore refers to an oligomer that hybridizes specifically to a target sequence in a nucleic acid, or in an amplified nucleic acid, under conditions that promote hybridization to allow selective detection of the target sequence.

A primer nucleic acid can be labeled, if desired, by incorporating a label detectable by, e.g., spectroscopic, photochemical, biochemical, immunochemical, chemical, or other techniques. To illustrate, useful labels include radioisotopes, fluorescent dyes, electron-dense reagents, enzymes (as commonly used in ELISAs), biotin, or haptens and proteins for which antisera or monoclonal antibodies are available. Many of these and other labels are described further herein and/or are otherwise known in the art. One of skill in the art will recognize that, in certain embodiments, primer nucleic acids can also be used as probe nucleic acids.

“RNA-dependent DNA polymerase” or “reverse transcriptase” (“RT”) refers to an enzyme that synthesizes a complementary DNA copy from an RNA template. All known reverse transcriptases also have the ability to make a complementary DNA copy from a DNA template; thus, they are both RNA- and DNA-dependent DNA polymerases. RTs may also have an RNAse H activity. A primer is required to initiate synthesis with both RNA and DNA templates.

“DNA-dependent DNA polymerase” is an enzyme that synthesizes a complementary DNA copy from a DNA template. Examples are DNA polymerase I from E. coli, bacteriophage T7 DNA polymerase, or DNA polymerases from bacteriophages T4, Phi-29, M2, or T5. DNA-dependent DNA polymerases may be the naturally occurring enzymes isolated from bacteria or bacteriophages or expressed recombinantly, or may be modified or “evolved” forms which have been engineered to possess certain desirable characteristics, e.g., thermostability, or the ability to recognize or synthesize a DNA strand from various modified templates. All known DNA-dependent DNA polymerases require a complementary primer to initiate synthesis. It is known that under suitable conditions a DNA-dependent DNA polymerase may synthesize a complementary DNA copy from an RNA template. RNA-dependent DNA polymerases typically also have DNA-dependent DNA polymerase activity.

“DNA-dependent RNA polymerase” or “transcriptase” is an enzyme that synthesizes multiple RNA copies from a double-stranded or partially double-stranded DNA molecule having a promoter sequence that is usually double-stranded. The RNA molecules (“transcripts”) are synthesized in the 5′-to-3′ direction beginning at a specific position just downstream of the promoter. Examples of transcriptases are the DNA-dependent RNA polymerase from E. coli and bacteriophages T7, T3, and SP6.

A “sequence” of a nucleic acid refers to the order and identity of nucleotides in the nucleic acid. A sequence is typically read in the 5′ to 3′ direction. The terms “identical” or percent “identity” in the context of two or more nucleic acid or polypeptide sequences, refer to two or more sequences or subsequences that are the same or have a specified percentage of amino acid residues or nucleotides that are the same, when compared and aligned for maximum correspondence, e.g., as measured using one of the sequence comparison algorithms available to persons of skill or by visual inspection. Exemplary algorithms that are suitable for determining percent sequence identity and sequence similarity are the BLAST programs, which are described in, e.g., Altschul et al. (1990) “Basic local alignment search tool” J. Mol. Biol. 215:403-410, Gish et al. (1993) “Identification of protein coding regions by database similarity search” Nature Genet. 3:266-272, Madden et al. (1996) “Applications of network BLAST server” Meth. Enzymol. 266:131-141, Altschul et al. (1997) “Gapped BLAST and PSI-BLAST: a new generation of protein database search programs” Nucleic Acids Res. 25:3389-3402, and Zhang et al. (1997) “PowerBLAST: A new network BLAST application for interactive or automated sequence analysis and annotation” Genome Res. 7:649-656, which are each incorporated by reference. Many other optimal alignment algorithms are also known in the art and are optionally utilized to determine percent sequence identity.

A “label” refers to a moiety attached (covalently or non-covalently), or capable of being attached, to a molecule, which moiety provides or is capable of providing information about the molecule (e.g., descriptive, identifying, etc. information about the molecule) or another molecule with which the labeled molecule interacts (e.g., hybridizes, etc.). Exemplary labels include fluorescent labels (including, e.g., quenchers or absorbers), weakly fluorescent labels, non-fluorescent labels, colorimetric labels, chemiluminescent labels, bioluminescent labels, radioactive labels, mass-modifying groups, antibodies, antigens, biotin, haptens, enzymes (including, e.g., peroxidase, phosphatase, etc.), and the like.

A “linker” refers to a chemical moiety that covalently or non-covalently attaches a compound or substituent group to another moiety, e.g., a nucleic acid, an oligonucleotide probe, a primer nucleic acid, an amplicon, a solid support, or the like. For example, linkers are optionally used to attach oligonucleotide probes to a solid support (e.g., in a linear or other logic probe array). To further illustrate, a linker optionally attaches a label (e.g., a fluorescent dye, a radioisotope, etc.) to an oligonucleotide probe, a primer nucleic acid, or the like. Linkers are typically at least bifunctional chemical moieties and in certain embodiments, they comprise cleavable attachments, which can be cleaved by, e.g., heat, an enzyme, a chemical agent, electromagnetic radiation, etc. to release materials or compounds from, e.g., a solid support. A careful choice of linker allows cleavage to be performed under appropriate conditions compatible with the stability of the compound and assay method. Generally a linker has no specific biological activity other than to, e.g., join chemical species together or to preserve some minimum distance or other spatial relationship between such species. However, the constituents of a linker may be selected to influence some property of the linked chemical species such as three-dimensional conformation, net charge, hydrophobicity, etc. Exemplary linkers include, e.g., oligopeptides, oligonucleotides, oligopolyamides, oligoethyleneglycerols, oligoacrylamides, alkyl chains, or the like. Additional description of linker molecules is provided in, e.g., Hermanson, Bioconjugate Techniques, Elsevier Science (1996), Lyttle et al. (1996) Nucleic Acids Res. 24(14):2793, Shchepino et al. (2001) Nucleosides, Nucleotides, & Nucleic Acids 20:369, Doronina et al (2001) Nucleosides, Nucleotides, & Nucleic Acids 20:1007, Trawick et al. (2001) Bioconjugate Chem. 12:900, Olejnik et al. (1998) Methods in Enzymology 291:135, and Pljevaljcic et al. (2003) J. Am. Chem. Soc. 125(12):3486, all of which are incorporated by reference.

“Fragment” refers to a piece of contiguous nucleic acid that contains fewer nucleotides than the complete nucleic acid.

“Hybridization,” “annealing,” “selectively bind,” or “selective binding” refers to the base-pairing interaction of one nucleic acid with another nucleic acid (typically an antiparallel nucleic acid) that results in formation of a duplex or other higher-ordered structure (i.e. a hybridization complex). The primary interaction between the antiparallel nucleic acid molecules is typically base specific, e.g., A/T and G/C. It is not a requirement that two nucleic acids have 100% complementarity over their full length to achieve hybridization. Nucleic acids hybridize due to a variety of well characterized physio-chemical forces, such as hydrogen bonding, solvent exclusion, base stacking and the like. An extensive guide to the hybridization of nucleic acids is found in Tijssen (1993) Laboratory Techniques in Biochemistry and Molecular Biology—Hybridization with Nucleic Acid Probes part I chapter 2, “Overview of principles of hybridization and the strategy of nucleic acid probe assays,” (Elsevier, New York), as well as in Ausubel (Ed.) Current Protocols in Molecular Biology, Volumes I, II, and III, 1997, which is incorporated by reference.

The term “attached” or “conjugated” refers to interactions and/or states in which material or compounds are connected or otherwise joined with one another. These interactions and/or states are typically produced by, e.g., covalent bonding, ionic bonding, chemisorption, physisorption, and combinations thereof.

A “composition” refers to a combination of two or more different components. In certain embodiments, for example, a composition includes one or more oligonucleotide probes in solution.

“Nucleic acid” or “nucleic acid molecule” refers to a multimeric compound comprising two or more covalently bonded nucleosides or nucleoside analogs having nitrogenous heterocyclic bases, or base analogs, where the nucleosides are linked together by phosphodiester bonds or other linkages to form a polynucleotide. Nucleic acids include RNA, DNA, or chimeric DNA-RNA polymers or oligonucleotides, and analogs thereof. A nucleic acid backbone can be made up of a variety of linkages, including one or more of sugar-phosphodiester linkages, peptide-nucleic acid bonds, phosphorothioate linkages, methylphosphonate linkages, or combinations thereof. Sugar moieties of the nucleic acid can be ribose, deoxyribose, or similar compounds having known substitutions (e.g. 2′-methoxy substitutions and 2′-halide substitutions). Nitrogenous bases can be conventional bases (A, G, C, T, U) or analogs thereof (e.g., inosine, 5-methylisocytosine, isoguanine).

An “oligonucleotide” or “oligomer” refers to a nucleic acid that includes at least two nucleic acid monomer units (e.g., nucleotides), typically more than three monomer units, and more typically greater than ten monomer units. The exact size of an oligonucleotide generally depends on various factors, including the ultimate function or use of the oligonucleotide. Oligonucleotides are optionally prepared by any suitable method, including, but not limited to, isolation of an existing or natural sequence, DNA replication or amplification, reverse transcription, cloning and restriction digestion of appropriate sequences, or direct chemical synthesis by a method such as the phosphotriester method of Narang et al. (1979) Meth. Enzymol. 68:90-99; the phosphodiester method of Brown et al. (1979) Meth. Enzymol. 68:109-151; the diethylphosphoramidite method of Beaucage et al. (1981) Tetrahedron Lett. 22:1859-1862; the triester method of Matteucci et al. (1981) J. Am. Chem. Soc. 103:3185-3191; automated synthesis methods; or the solid support method of U.S. Pat. No. 4,458,066, or other methods known in the art. All of these references are incorporated by reference.

A “mixture” refers to a combination of two or more different components. A “reaction mixture” refers a mixture that comprises molecules that can participate in and/or facilitate a given reaction. An “amplification reaction mixture” refers to a solution containing reagents necessary to carry out an amplification reaction, and typically contains primers, a thermostable DNA polymerase, dNTP's, and a divalent metal cation in a suitable buffer. A reaction mixture is referred to as complete if it contains all reagents necessary to carry out the reaction, and incomplete if it contains only a subset of the necessary reagents. It will be understood by one of skill in the art that reaction components are routinely stored as separate solutions, each containing a subset of the total components, for reasons of convenience, storage stability, or to allow for application-dependent adjustment of the component concentrations, and, that reaction components are combined prior to the reaction to create a complete reaction mixture. Furthermore, it will be understood by one of skill in the art that reaction components are packaged separately for commercialization and that useful commercial kits may contain any subset of the reaction components, which includes the modified primers of the invention.

### Examples

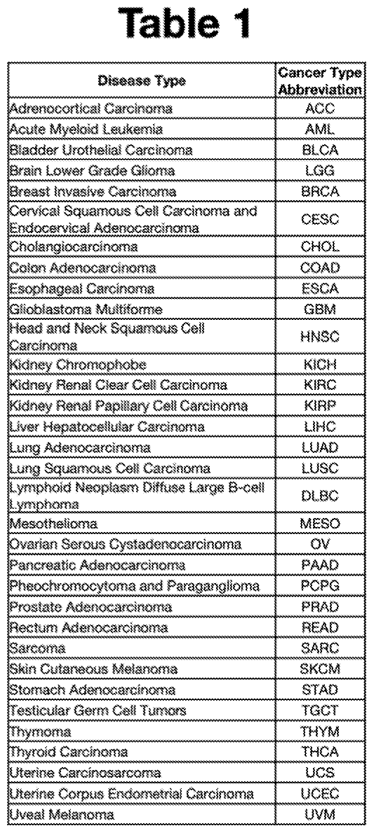

The broad evaluation of microbes from cancer patient sequencing data is shown in FIG. 1A across 33 cancer types in TCGA. Since these data derived from multiple sequencing centers, they had to be batch corrected (FIGS. 1B-1C), which was done in a supervised manner, permitting selective reduction of technical batch variables while retaining or increasing the importance of biological variables (FIG. 1D).

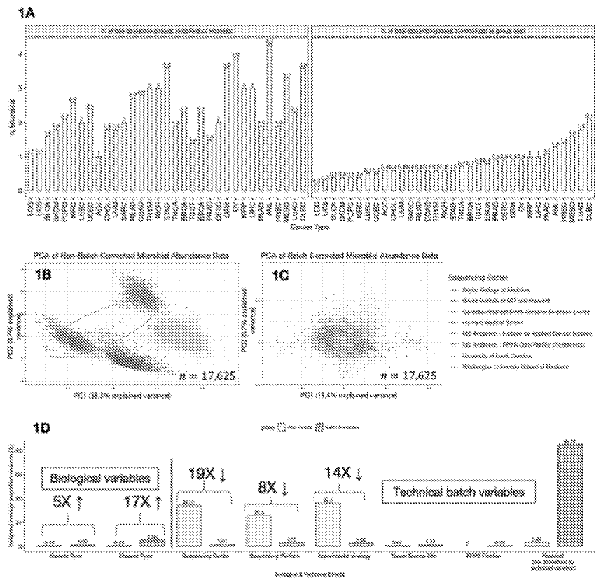

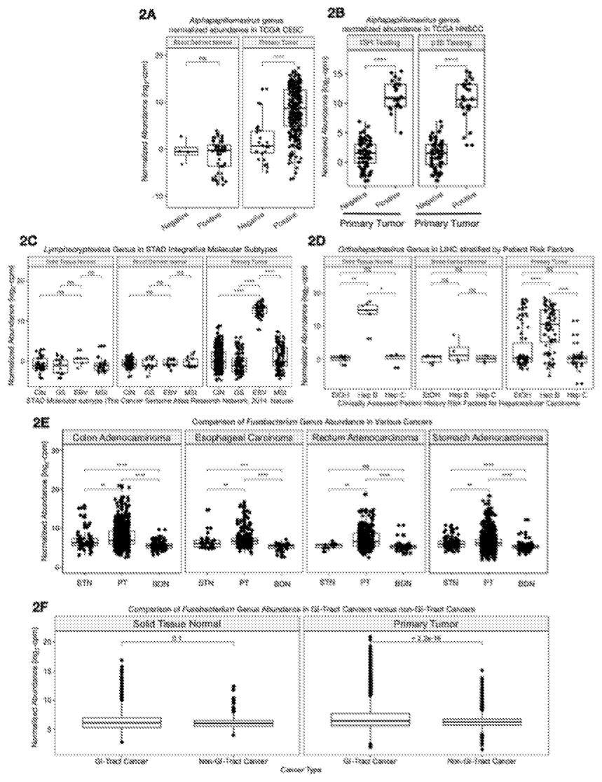

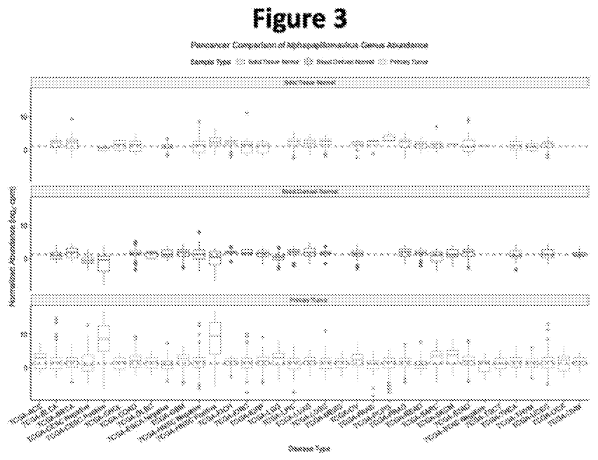

Ecological validation was subsequently performed to ensure that the identified microbes were in line with expected and/or observed clinical and literature findings (FIGS. 2A-3).

Concurrently, another dataset from Hugo et al. (2016; Science; PMID: 26997480) that collected whole transcriptomic data from patients' tumors prior to them receiving anti-PD-1 immunotherapy (i.e. nivolumab or pembrolizumab) was harvested for microbial reads. The intratumoral microbial RNA was then used to distinguish patients who had a ‘complete response’ (CR) versus those who had ‘progressive disease’ (PD), per iRECIST classification, as well as to distinguish patients who had a ‘complete response’ (CR) versus those who had a ‘partial response’ (PR). The PCoA plots are shown in FIGS. 4A and 4C, and the plots showing discriminatory beta dispersion differences between the comparisons are shown in FIGS. 4B and 4D.

Since the concept of immunogenicity is important in predicting response to certain types of cancer therapy, immunogenic subtypes of cancers were explored in TCGA to see if they could be discriminated by microbial DNA and RNA against non-immunogenic subtypes of cancer. Presented examples herein include discriminating cases of microsatellite instability in colon cancer (FIG. 4E) and discriminating cases of triple negative (“basal-like”) subtype of breast cancer among other breast cancer subtypes (FIG. 4F).

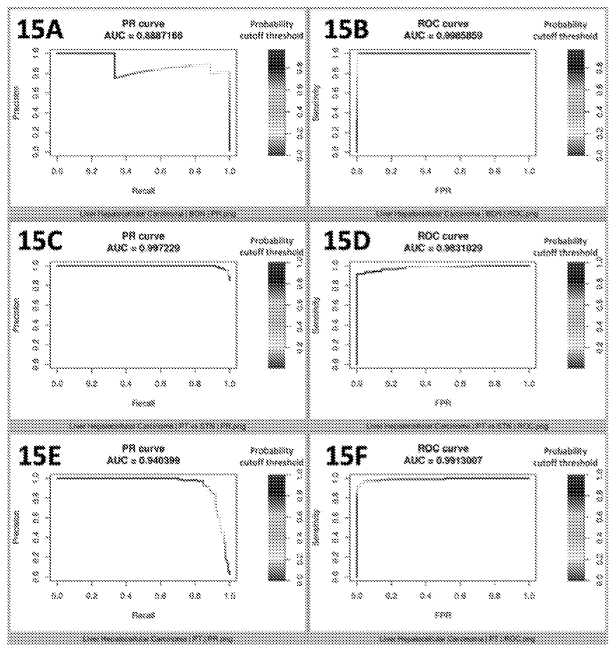

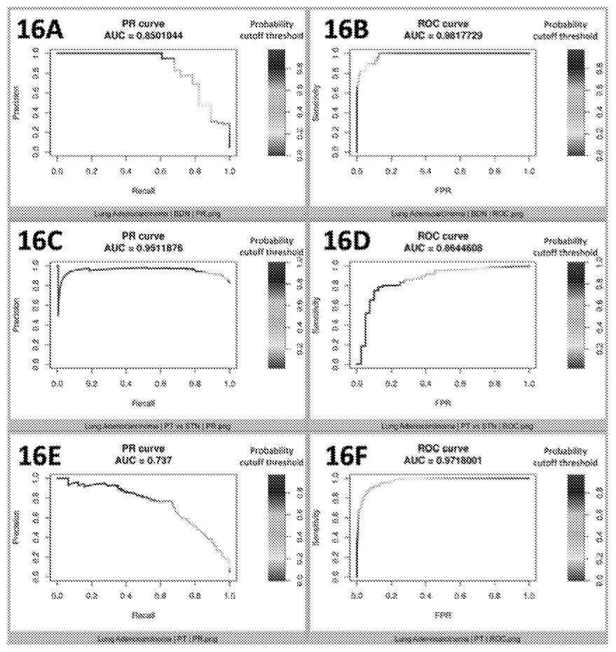

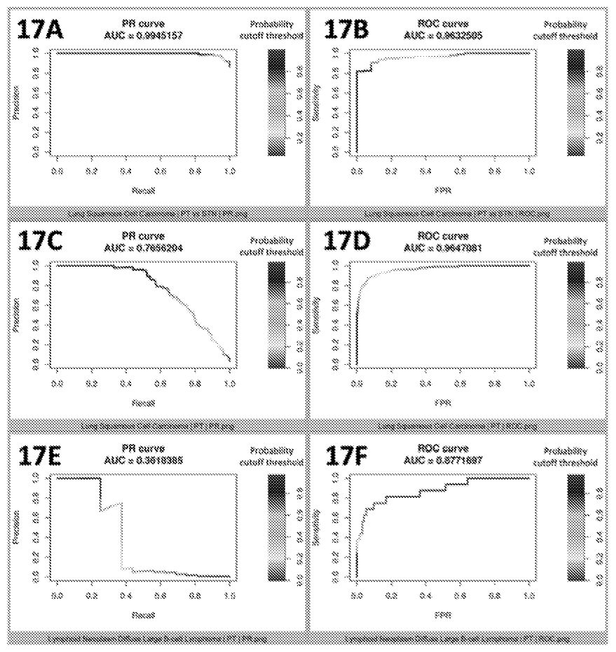

Using liver hepatocellular carcinoma as an example for distinguishing primary tumor samples as coming from a particular cancer type by solely using microbial DNA and RNA, a total of 13,883 primary tumor samples were processed across 32 cancer types, 416 of which were liver cancer. After training on a randomly selected, class-stratified 70% of the cases and testing on the remaining 30% cases, the model showed nearly perfect discrimination with an area under the receiver operator curve (AUROC) of 0.991300703 and an area under the precision-recall curve (AUPR) of 0.940399017. FIGS. 15E and 16F shows the PR and ROC curves, respectively, of the model's performance on the randomly selected 30% holdout test set. The model performance is also shown in the website screenshot in FIG. 33B.

Using liver hepatocellular carcinoma as another example for distinguishing blood-derived normal samples as coming from a particular cancer type by solely using microbial DNA, a total of 1866 blood-derived normal samples were processed, 32 of which were from liver cancer. After training on a randomly selected, class-stratified 70% of the cases, the model was tested on the remaining 30% of the cases and showed exceptionally good discrimination with an AUROC of 0.998585859 and an AUPR of 0.888716603. The respective PR and ROC plots are shown in FIGS. 15A and 15B.

Again using liver hepatocellular carcinoma as another example for distinguishing tumor tissue from normal tissue solely using microbial DNA and RNA, all of the primary tumor and adjacent solid tissue normal samples from liver cancer patients were extracted for processing (n=488, of which 416 are primary tumors and 72 are adjacent solid tissue normals). After training on a randomly selected 70% of the cases, the model was tested on the remaining 30% of the cases and showed phenomenal discrimination with an AUROC of 0.983102919 and an AUPR of 0.997228962. The respective PR and ROC plots are shown in FIGS. 15C and 15D.

A similar procedure, as described above, was applied to every possible discrimination for every cancer type in the TCGA dataset, as long as the minority class contained at least 20 samples, and are shown in FIGS. 5A-27B. The cancer types shown include the following: Adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, or uveal melanoma. Data on the discriminatory performance on acute myelogenous leukemia samples were shown in the provisional application but are not shown here.

In cases of class imbalance, up-sampling of the minority class was used to promote model generalization, as shown herein. Many other strategies were previously attempted and presented in the provisional application, including: differential weighting of the samples during model training (i.e. higher weighting of minority class and lower weighting of majority class); down sampling the majority class; and interpolating new instances of the minority class using several interpolation algorithms (i.e. SMOTE and ROSE). Minor variation in model performance is possible with these, and someone skilled in the art will anticipate ways to improve model performance by their implementation and fine-tuning. For example, some of these strategies lead to models of the same discrimination that differ substantially in their sensitivity versus specificity, and it is possible to combine these models into an ensemble to make an overall better performing model.

Notably, the models presented herein have been minimally tuned and there is an anticipated opportunity to increase their predictive accuracy, among other performance metrics, by further model tuning and/or employing different training strategies, increasing sample size, regularization, model types, building ensembles of models, or a combination thereof.

To study the effects of (de)contamination on the model predictions, a decontamination pipeline was theorized and implemented (FIG. 28A) prior to machine learning model building and testing. Notably, the decontamination pipeline described in FIG. 28A represents one among many ways to evaluate the impact of and remove contaminants from such cancer microbiome data, and an individual skilled in the art will be to anticipate other such methods that extend or lessen the complexity of the presented pipeline. After decontamination, FIGS. 28B and 28C show that classifier performance is maintained relative to models built and tested on the “full dataset” that was not decontaminated.

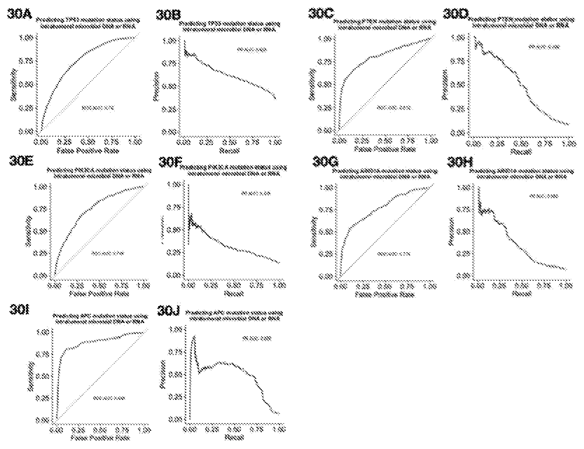

In order to explore the generality of the findings described herein, several additional steps of analysis were performed. The first split the original microbial count data in half in a stratified manner, then normalized and batch corrected each half independently, and then built separate machine learning models on each half. The trained machine learning model was then tested on the opposite half's data to estimate overall performance and model generalization. These predictions involved labeling one cancer type versus all others solely using microbial DNA and RNA from primary tumors. These performance values were then compared to a model trained and tested on the full dataset that had been normalized and batch corrected with 50%-50% training-testing splits, also predicting one cancer type versus all others solely using microbial DNA and RNA from primary tumors. The results are shown in FIG. 29A. Additionally, further comparative analysis on models built and tested on RNA-only data (FIGS. 29B-29C) or DNA-only data (FIGS. 29D-29E) did not show significant reductions in overall model performance. Even a more stringent comparative analysis, whereby data from a single sequencing center that only performed one type of sequencing (University of North Carolina: RNA-Seq) or another (Harvard Medical School: whole genome sequencing) were used to train and test models, did not show significant reductions in predictive performance when predicting one cancer type versus all others solely based on microbial nucleic acid information (FIGS. 29F-29I).

FIG. 30 shows several examples of predicting the mutation status of the top five most common mutations in TCGA solely using microbial DNA and RNA in primary tumors in a pan-cancer fashion.

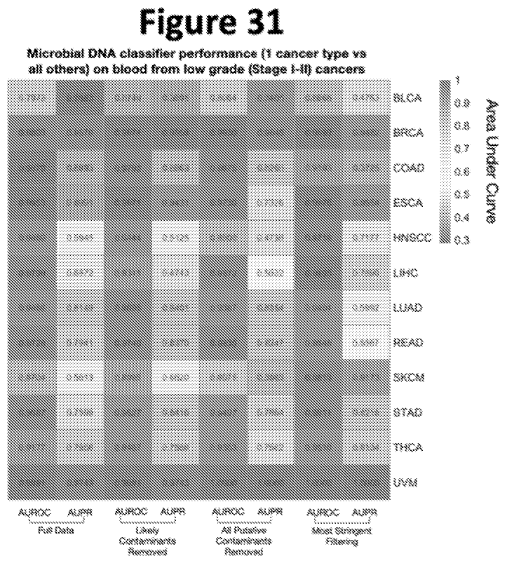

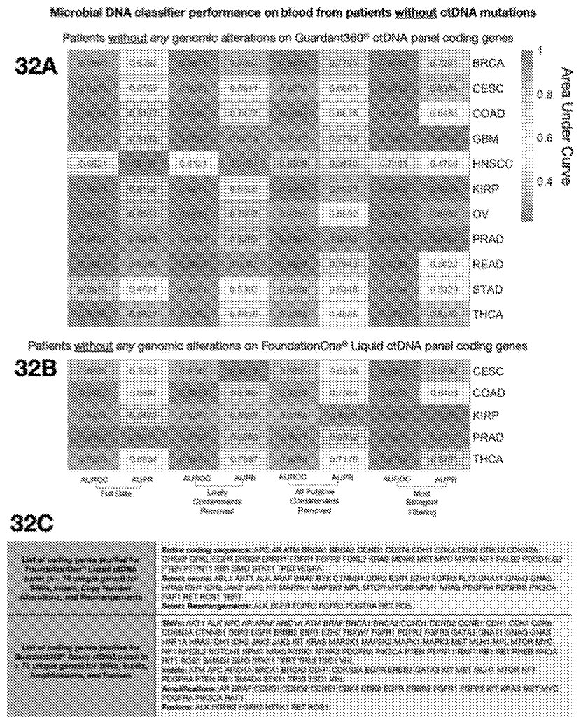

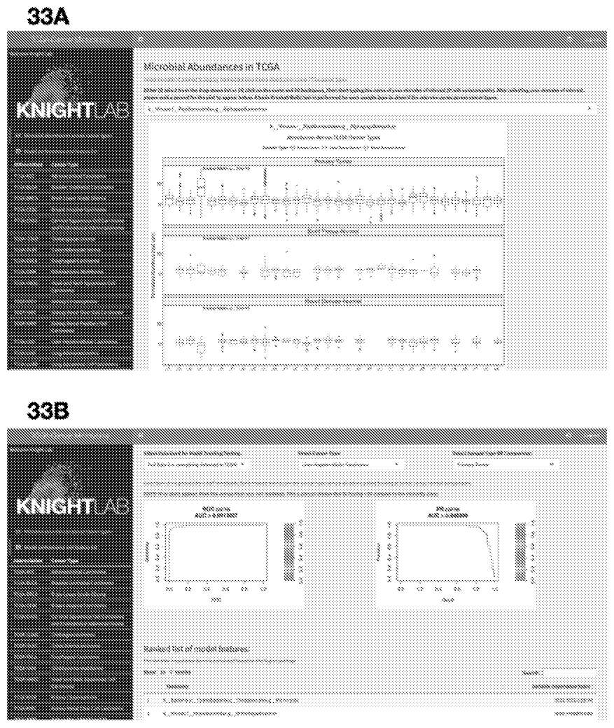

Since many currently available liquid biopsy diagnostics are not able to accurately diagnose low-stage cancers (stage I and stage II), a conservative benchmarking analysis was done using microbial DNA derived from blood samples of TCGA patients who only had stage I or stage II cancers. FIG. 31 shows that it is readily feasible to distinguish which cancer type a given blood sample belong to solely using microbial DNA and further shows that varying stringencies of decontamination do not drastically affect the performance of the model classifications.

FIG. 32 also depicts a very conservative benchmarking analysis for predicting cancer type using microbial DNA derived from blood samples of TCGA patients that do not have any detectable genomic alterations in their tumors as measured by two commercial ctDNA assays. The results show that it is readily feasible to distinguish which cancer type a given blood sample belongs to just based on the microbial DNA found within it, notably when two major liquid biopsy assays would fail to even detect the presence of cancer, even when assuming 100% sensitivity and 100% specificity.

FIG. 33 describes how an electronic website interface can be built for hosting, displaying, and sharing information about microbial presence and abundance in various cancer types, as well as showing model performances and which microbial features were most important for a model to make a particular discrimination. For anyone skilled in the art, it is expected that similar electronic, online interfaces can be used to remotely evaluate and diagnose a cancer using microbial nucleic acids that were measured as part of a deployable kit.

Appendix A is a listing of microbial features (i.e. taxonomy names at the genus level) that were detected in TCGA (n=1993). The models presented herein were not regularized and can utilize information from all 1993 available genera, although many models performed well with 30-1200 genera. Furthermore, a number of “decontaminated” datasets were built off of this original “full dataset” with varying levels of decontamination stringency. Since the combinatorial number of models trained and tested on all possible comparisons and datasets is high, and since the number of genera per model is even higher (i.e. several to many genera per model), it is not necessary to list out every ranked, unique model feature (estimated at >120,000 features) in this patent application. Instead, it is expected that someone skilled in the art would be able to readily replicate the invention using the methods described herein, as well as the list of microbial features provided. It is further expected that any subset of these microbial features, as selected by some algorithmic or machine learning process, can be used to make a variety of discriminatory predictions among various cancer types, subtypes, mutation statuses, samples types, treatment responses, and so forth.

The diagnostic methods described herein further provide a basis for methods of treatment of a diagnosed subject with an effective amount of a therapy directed against the diagnosed cancer, wherein the therapy now known in the art or later discovered.

An example of analogous machine learning model creation known to those in the art is Ridgeway, “Generalized Boosted Models: a guide to the gbm package” 2007, as well as in Kuhn, Max, and Kjell Johnson, Applied predictive modeling. Vol. 26. New York: Springer, 2013, incorporated herein by reference.

These and other aspects features, alternatives and advantages of the present invention will be apparent to those skilled in the art upon a review of the specific embodiments disclosed herein, which are not to be considered limiting to the scope of the claimed invention.

APPENDIX A
k_Viruses.f_Phycodnaviridae.g_Prasinovirus
k_Viruses.o_Caudovirales.f_Siphoviridae.g_Sfi1unalikevirus
k_Viruses.o_Herpesvirales.f_Herpesviridae.g_Simplexvirus
k_Viruses.o_Caudovirales.f_Siphoviridae.g_C2likevirus
k_Viruses.f_Poxviridae.g_Parapoxvirus
k_Viruses.f_Papillomaviridae.g_Dyolambdapapillomavirus
k_Viruses.o_Caudovirales.f_Siphoviridae.g_Bignuzlikevirus
k_Viruses.o_Mononegavirales.f_Bornaviridae.g_Bornavirus
k_Viruses.f_Bicaudaviridae.g_Bicaudavirus
k_Viruses.o_Picornavirales.f_Picornaviridae.g_Aquamavirus
k_Viruses.o_Herpesvirales.f_Herpesviridae.g_Scutavirus
k_Viruses.o_Herpesvirales.f_Herpesviridae.g_Mardivirus
k_Viruses.f_Baculoviridae.g_Deltabaculovirus
k_Viruses.f_Papillomaviridae.g_Taupapillomavirus
k_Viruses.o_Caudovirales.f_Myoviridae.g_I3likevirus
k_Viruses.f_Virgaviridae.g_Furovirus
k_Viruses.o_Herpesvirales.f_Herpesviridae.g_Proboscivirus
k_Viruses.o_Herpesvirales.f_Alloherpesviridae.g_Ictalurivirus
k_Viruses.f_Inoviridae.g_Inovirus
k_Viruses.f_Marseilleviridae.g_Marseillevirus
k_Viruses.o_Nidovirales.f_Coronaviridae.g_Gammacoronavirus
k_Viruses.f_Anelloviridae.g_Thetatorquevirus
k_Viruses.f_Ascoviridae.g_Ascovirus
k_Viruses.o_Tymovirales.f_Tymoviridae.g_Maculavirus
k_Viruses.g_Negevirus
k_Viruses.o_Caudovirales.f_Myoviridae.g_Spo1virus
k_Viruses.f_Circoviridae.g_Gyrovirus
k_Viruses.o_Mononegavirales.f_Rhabdoviridae.g_Novirhabdovirus
k_Viruses.o_Picornavirales.f_Picornaviridae.g_Sicinivirus
k_Viruses.o_Mononegavirales.g_Pneumovirus
k_Viruses.o_Picornavirales.f_Picornaviridae.g_Dicipivirus
k_Viruses.f_Poxviridae.g_Molluscipoxvirus
k_Viruses.o_Tymovirales.f_Tymoviridae.g_Tymovirus
k_Viruses.f_Arenaviridae.g_Mammarenavirus
k_Viruses.f_Totiviridae.g_Victorivirus
k_Viruses.f_Flaviviridae.g_Hepacivirus
k_Viruses.f_Poxviridae.g_Alphaentomopoxvirus
k_Viruses.f_Anelloviridae.g_Gammatorquevirus
k_Viruses.o_Tymovirales.f_Gammaflexiviridae.g_Mycoflexivirus
k_Viruses.g_Tenuivirus
k_Viruses.f_Hepeviridae.g_Piscihepevirus
k_Viruses.f_Virgaviridae.g_Pomovirus
k_Viruses.f_Caulimoviridae.g_Badnavirus
k_Viruses.f_Polydnaviridae.g_Ichnovirus
k_Viruses.o_Herpesvirales.f_Herpesviridae.g_Percavirus
k_Viruses.f_Papillomaviridae.g_Betapapillomavirus
k_Viruses.f_Togaviridae.g_Alphavirus
k_Viruses.o_Tymovirales.f_Betaflexiviridae.g_Trichovirus
k_Viruses.o_Caudovirales.f_Podoviridae.g_Bcep22likevirus
k_Viruses.o_Tymovirales.f_Alphaflexiviridae.g_Sclerodarnavirus
k_Viruses.f_Phycodnaviridae.g_Coccolithovirus
k_Viruses.o_Ligamenvirales.f_Lipothrixviridae.g_Deltalipothrixvirus
k_Viruses.f_Papillomaviridae.g_Omikronpapillomavirus
k_Viruses.f_Retroviridae.g_Alpharetrovirus
k_Viruses.o_Caudovirales.f_Siphoviridae.g_Jerseylikevirus
k_Viruses.f_Poxviridae.g_Capripoxvirus
k_Viruses.f_Poxviridae.g_Cervidpoxvirus
k_Viruses.g_Higrevirus
k_Viruses.f_Potyviridae.g_Bymovirus
k_Viruses.o_Caudovirales.f_Podoviridae.g_Phikmvlikevirus
k_Viruses.o_Picornavirales.f_Picornaviridae.g_Tremovirus
k_Viruses.o_Picornavirales.f_Secoviridae.g_Torradovirus
k_Viruses.o_Caudovirales.f_Siphoviridae.g_T5likevirus
k_Viruses.o_Nidovirales.f_Coronaviridae.g_Bafinivirus
k_Viruses.f_Baculoviridae.g_Alphabaculovirus
k_Viruses.f_Papillomaviridae.g_Mupapillomavirus
k_Viruses.o_Caudovirales.f_Siphoviridae.g_Lambdalikevirus
k_Viruses.o_Caudovirales.f_Myoviridae.g_Felixounalikevirus
k_Viruses.f_Papillomaviridae.g_Phipapillomavirus
k_Viruses.f_Papillomaviridae.g_Omegapapillomavirus
k_Viruses.o_Nidovirales.f_Coronaviridae.g_Betacoronavirus
k_Viruses.f_Luteoviridae.g_Enamovirus
k_Viruses.f_Phycodnaviridae.g_Chlorovirus
k_Viruses.f_Hypoviridae.g_Hypovirus
k_Viruses.f_Anelloviridae.g_Alphatorquevirus
k_Viruses.o_Picornavirales.f_Dicistroviridae.g_Aparavirus
k_Viruses.g_Pithovirus
k_Viruses.f_Phycodnaviridae.g_Phaeovirus
k_Viruses.o_Picornavirales.f_Picornaviridae.g_Hepatovirus
k_Viruses.f_Virgaviridae.g_Tobamovirus
k_Viruses.o_Caudovirales.f_Siphoviridae.g_Barnyardlikevirus
k_Viruses.f_Baculoviridae.g_Betabaculovirus
k_Viruses.f_Arenaviridae.g_Arenavirus
k_Viruses.o_Picornavirales.f_Marnaviridae.g_Marnavirus
k_Viruses.o_Caudovirales.f_Myoviridae.g_P100virus
k_Viruses.o_Caudovirales.f_Myoviridae.g_Spounalikevirus
k_Viruses.f_Partitiviridae.g_Betapartitivirus
k_Viruses.f_Reoviridae.g_Cypovirus
k_Viruses.o_Tymovirales.f_Betaflexiviridae.g_Carlavirus
k_Viruses.o_Nidovirales.f_Coronaviridae.g_Alphacoronavirus
k_Viruses.o_Mononegavirales.f_Paramyxoviridae.g_Avulavirus
k_Viruses.o_Herpesvirales.f_Herpesviridae.g_Iltovirus
k_Viruses.f_Bromoviridae.g_Bromovirus
k_Viruses.f_Bunyaviridae.g_Phlebovirus
k_Viruses.f_Closteroviridae.g_Closterovirus
k_Viruses.o_Caudovirales.f_Myoviridae.g_Viunalikevirus
k_Viruses.o_Caudovirales.f_Siphoviridae.g_Phicbklikevirus
k_Viruses.f_Fuselloviridae.g_Alphafusellovirus
k_Viruses.f_Bunyaviridae.g_Orthobunyavirus
k_Viruses.o_Picornavirales.f_Picornaviridae.g_Sapelovirus
k_Viruses.f_Bunyaviridae.g_Tospovirus
k_Viruses.f_Mimiviridae.g_Cafeteriavirus
k_Viruses.f_Poxviridae.g_Suipoxvirus
k_Viruses.f_Potyviridae.g_Potyvirus
k_Viruses.o_Herpesvirales.f_Herpesviridae.g_Muromegalovirus
k_Viruses.o_Picornavirales.f_Picornaviridae.g_Salivirus
k_Viruses.o_Caudovirales.f_Podoviridae.g_N4likevirus
k_Viruses.f_Retroviridae.g_Lentivirus
k_Viruses.o_Picornavirales.f_Secoviridae.g_Fabavirus
k_Viruses.f_Polydnaviridae.g_Bracovirus
k_Viruses.o_Herpesvirales.f_Herpesviridae.g_Rhadinovirus
k_Viruses.f_Luteoviridae.g_Polerovirus
k_Viruses.f_Flaviviridae.g_Pestivirus
k_Viruses.f_Poxviridae.g_Betaentomopoxvirus
k_Viruses.f_Geminiviridae.g_Begomovirus
k_Viruses.o_Herpesvirales.f_Alloherpesviridae.g_Cyprinivirus
k_Viruses.o_Mononegavirales.f_Nyamiviridae.g_Nyavirus
k_Viruses.f_Potyviridae.g_Rymovirus
k_Viruses.f_Retroviridae.g_Betaretrovirus
k_Viruses.f_Microviridae.g_Microvirus
k_Viruses.o_Caudovirales.f_Siphoviridae.g_Andromedalikevirus
k_Viruses.f_Iridoviridae.g_Chloriridovirus
k_Viruses.o_Picornavirales.f_Picornaviridae.g_Aphthovirus
k_Viruses.f_Iridoviridae.g_Lymphocystivirus
k_Viruses.o_Tymovirales.f_Alphaflexiviridae.g_Lolavirus
k_Viruses.o_Caudovirales.f_Myoviridae.g_Silviavirus
k_Viruses.f_Caliciviridae.g_Vesivirus
k_Viruses.f_Poxviridae.g_Avipoxvirus
k_Viruses.g_Cilevirus
k_Viruses.o_Mononegavirales.f_Paramyxoviridae.g_Rubulavirus
k_Viruses.f_Chrysoviridae.g_Chrysovirus
k_Viruses.o_Caudovirales.f_Myoviridae.g_Twortlikevirus
k_Viruses.g_Emaravirus
k_Viruses.f_Astroviridae.g_Avastrovirus
k_Viruses.o_Picornavirales.f_Secoviridae.g_Cheravirus
k_Viruses.o_Ligamenvirales.f_Rudiviridae.g_Rudivirus
k_Viruses.o_Herpesvirales.f_Herpesviridae.g_Varicellovirus
k_Viruses.f_Polyomaviridae.g_Polyomavirus
k_Viruses.o_Herpesvirales.f_Alloherpesviridae.g_Batrachovirus
k_Viruses.o_Picornavirales.f_Picornaviridae.g_Enterovirus
k_Viruses.o_Picornavirales.f_Secoviridae.g_Waikavirus
k_Viruses.o_Caudovirales.f_Myoviridae.g_T4likevirus
k_Viruses.f_Poxviridae.g_Yatapoxvirus
k_Viruses.f_Adenoviridae.g_Mastadenovirus
k_Viruses.o_Herpesvirales.f_Herpesviridae.g_Cytomegalovirus
k_Viruses.o_Picornavirales.f_Picornaviridae.g_Kobuvirus
k_Viruses.o_Caudovirales.f_Podoviridae.g_T7likevirus
k_Viruses.o_Herpesvirales.f_Herpesviridae.g_Roseolovirus
k_Viruses.f_Adenoviridae.g_Aviadenovirus
k_Viruses.o_Ligamenvirales.f_Lipothrixviridae.g_Betalipothrixvirus
k_Viruses.f_Iridoviridae.g_Ranavirus
k_Viruses.f_Closteroviridae.g_Ampelovirus
k_Viruses.o_Caudovirales.f_Siphoviridae.g_Phietalikevirus
k_Viruses.o_Caudovirales.f_Siphoviridae.g_Pgonelikevirus
k_Viruses.f_Orthomyxoviridae.g_Isavirus
k_Viruses.f_Poxviridae.g_Orthopoxvirus
k_Viruses.f_Papillomaviridae.g_Gammapapillomavirus
k_Viruses.f_Phycodnaviridae.g_Prymnesiovirus
k_Viruses.f_Bunyaviridae.g_Hantavirus
k_Viruses.o_Picornavirales.f_Picornaviridae.g_Senecavirus
k_Viruses.o_Caudovirales.f_Siphoviridae.g_L5likevirus
k_Viruses.o_Picornavirales.f_Dicistroviridae.g_Cripavirus
k_Viruses.o_Caudovirales.f_Siphoviridae.g_Tunalikevirus
k_Viruses.f_Hytrosaviridae.g_Muscavirus
k_Viruses.o_Herpesvirales.f_Herpesviridae.g_Macavirus
k_Viruses.f_Bromoviridae.g_Oleavirus
k_Viruses.f_Closteroviridae.g_Crinivirus
k_Viruses.f_Papillomaviridae.g_Lambdapapillomavirus
k_Viruses.f_Papillomaviridae.g_Alphapapillomavirus
k_Viruses.f_Partitiviridae.g_Alphapartitivirus
k_Viruses.f_Mimiviridae.g_Mimivirus
k_Viruses.o_Herpesvirales.f_Malacoherpesviridae.g_Ostreavirus
k_Viruses.o_Mononegavirales.g_Metapneumovirus
k_Viruses.o_Tymovirales.f_Alphaflexiviridae.g_Potexvirus
k_Viruses.o_Caudovirales.f_Siphoviridae.g_Sfi21dtunalikevirus
k_Viruses.f_Retroviridae.g_Gammaretrovirus
k_Viruses.f_Circoviridae.g_Circovirus
k_Viruses.o_Tymovirales.f_Betaflexiviridae.g_Foveavirus
k_Viruses.o_Picornavirales.f_Secoviridae.g_Nepovirus
k_Viruses.o_Picornavirales.f_Iflaviridae.g_Iflavirus
k_Viruses.f_Iridoviridae.g_Iridovirus
k_Viruses.f_Flaviviridae.g_Flavivirus
k_Viruses.f_Astroviridae.g_Mamastrovirus
k_Viruses.f_Virgaviridae.g_Pecluvirus
k_Viruses.o_Nidovirales.f_Roniviridae.g_Okavirus
k_Viruses.g_Bacilladnavirus
k_Viruses.f_Bromoviridae.g_Cucumovirus
k_Viruses.o_Herpesvirales.f_Herpesviridae.g_Lymphocryptovirus
k_Viruses.f_Nimaviridae.g_Whispovirus
k_Viruses.f_Hytrosaviridae.g_Glossinavirus
k_Viruses.f_Virgaviridae.g_Hordeivirus
k_Viruses.o_Caudovirales.f_Siphoviridae.g_Skunalikevirus
k_Viruses.o_Caudovirales.f_Myoviridae.g_Cp220likevirus
k_Viruses.o_Tymovirales.f_Tymoviridae.g_Marafivirus
k_Viruses.o_Caudovirales.f_Myoviridae.g_Phikzlikevirus
k_Viruses.o_Caudovirales.f_Myoviridae.g_Cp8unalikevirus
k_Viruses.o_Caudovirales.f_Myoviridae.g_PhiCD119likevirus
k_Viruses.f_Papillomaviridae.g_Pipapillomavirus
k_Viruses.o_Picornavirales.f_Picornaviridae.g_Megrivirus
k_Viruses.o_Caudovirales.f_Myoviridae.g_Kayvirus
k_Viruses.o_Caudovirales.f_Siphoviridae.g_C5likevirus
k_Viruses.f_Endornaviridae.g_Endornavirus
k_Viruses.f_Reoviridae.g_Orbivirus
k_Viruses.f_Adenoviridae.g_Atadenovirus
k_Viruses.f_Orthomyxoviridae.g_Influenzavirus_C
k_Viruses.o_Tymovirales.f_Alphaflexiviridae.g_Botrexvirus
k_Viruses.f_Poxviridae.g_Leporipoxvirus
k_Viruses.o_Caudovirales.f_Podoviridae.g_Phi29likevirus
k_Viruses.f_Parvoviridae.g_Erythroparvovirus
k_Viruses.o_Caudovirales.f_Myoviridae.g_Schizot4likevirus
k_Viruses.f_Baculoviridae.g_Gammabaculovirus
k_Viruses.o_Caudovirales.f_Siphoviridae.g_Yualikevirus
k_Viruses.o_Picornavirales.f_Picornaviridae.g_Cosavirus
k_Viruses.f_Ampullaviridae.g_Ampullavirus
k_Viruses.f_Tectiviridae.g_Tectivirus
k_Viruses.f_Caulimoviridae.g_Cavemovirus
k_Viruses.f_Anelloviridae.g_Epsilontorquevirus
k_Viruses.f_Papillomaviridae.g_Deltapapillomavirus
k_Viruses.f_Parvoviridae.g_Iteradensovirus
k_Viruses.o_Mononegavirales.f_Rhabdoviridae.g_Ephemerovirus
k_Viruses.f_Poxviridae.g_Crocodylidpoxvirus
k_Viruses.f_Anelloviridae.g_Betatorquevirus
k_Viruses.f_Turriviridae.g_Alphaturrivirus
k_Viruses.f_Caulimoviridae.g_Caulimovirus
k_Viruses.o_Caudovirales.f_Siphoviridae.g_P23likevirus
k_Viruses.o_Caudovirales.f_Myoviridae.g_Pbunalikevirus
k_Viruses.f_Globuloviridae.g_Globulovirus
k_Viruses.f_Reoviridae.g_Fijivirus
k_Viruses.f_Reoviridae.g_Phytoreovirus
k_Viruses.f_Benyviridae.g_Benyvirus
k_Viruses.f_Parvoviridae.g_Protoparvovirus
k_Viruses.f_Asfarviridae.g_Asfivirus
k_Viruses.f_Luteoviridae.g_Luteovirus
k_Viruses.o_Mononegavirales.f_Paramyxoviridae.g_Respirovirus
k_Viruses.o_Mononegavirales.f_Filoviridae.g_Marburgvirus
k_Viruses.f_Fuselloviridae.g_Betafusellovirus
k_Viruses.o_Picornavirales.f_Picornaviridae.g_Pasivirus
k_Viruses.f_Bromoviridae.g_Alfamovirus
k_Viruses.o_Caudovirales.f_Podoviridae.g_Sp6likevirus
k_Viruses.o_Nidovirales.f_Coronaviridae.g_Deltacoronavirus
k_Viruses.f_Inoviridae.g_Plectrovirus
k_Viruses.o_Picornavirales.f_Secoviridae.g_Sadwavirus
k_Viruses.o_Mononegavirales.f_Paramyxoviridae.g_Morbillivirus
k_Viruses.o_Mononegavirales.g_Orthopneumovirus
k_Viruses.o_Caudovirales.f_Siphoviridae.g_3alikevirus
k_Viruses.o_Tymovirales.f_Alphaflexiviridae.g_Mandarivirus
k_Viruses.f_Togaviridae.g_Rubivirus
k_Viruses.o_Tymovirales.f_Betaflexiviridae.g_Vitivirus
k_Viruses.o_Caudovirales.f_Siphoviridae.g_Xp10likevirus
k_Viruses.f_Sphaerolipoviridae.g_Alphasphaerolipovirus
k_Viruses.o_Caudovirales.f_Myoviridae.g_Hpunalikevirus
k_Viruses.f_Parvoviridae.g_Brevidensovirus
k_Viruses.f_Papillomaviridae.g_Dyoetapapillomavirus
k_Viruses.o_Caudovirales.f_Siphoviridae.g_Sap6likevirus
k_Viruses.o_Caudovirales.f_Siphoviridae.g_Che9clikevirus
k_Viruses.f_Caulimoviridae.g_Rosadnavirus
k_Viruses.f_Caulimoviridae.g_Solendovirus
k_Viruses.f_Sphaerolipoviridae.g_Gammasphaerolipovirus
k_Viruses.o_Caudovirales.f_Siphoviridae.g_Phijlunalikevirus
k_Viruses.o_Caudovirales.f_Myoviridae.g_P2likevirus
k_Viruses.f_Flaviviridae.g_Pegivirus
k_Viruses.f_Adenoviridae.g_Siadenovirus
k_Viruses.f_Parvoviridae.g_Copiparvovirus
k_Viruses.f_Anelloviridae.g_Iotatorquevirus
k_Viruses.o_Caudovirales.f_Myoviridae.g_Hapunalikevirus
k_Viruses.o_Caudovirales.f_Siphoviridae.g_N15likevirus
k_Viruses.f_Retroviridae.g_Epsilonretrovirus
k_Viruses.o_Mononegavirales.f_Paramyxoviridae.g_Henipavirus
k_Viruses.f_Reoviridae.g_Rotavirus
k_Viruses.o_Ligamenvirales.f_Lipothrixviridae.g_Gammalipothrixvirus
k_Viruses.f_Reoviridae.g_Mimoreovirus
k_Viruses.o_Mononegavirales.f_Rhabdoviridae.g_Vesiculovirus
k_Viruses.o_Mononegavirales.f_Rhabdoviridae.g_Lyssavirus
k_Viruses.f_Hepadnaviridae.g_Orthohepadnavirus
k_Viruses.f_Parvoviridae.g_Dependoparvovirus
k_Viruses.o_Tymovirales.f_Alphaflexiviridae.g_Allexivirus
k_Viruses.o_Picornavirales.f_Picornaviridae.g_Parechovirus
k_Viruses.o_Nidovirales.f_Mesoniviridae.g_Alphamesonivirus
k_Viruses.o_Caudovirales.f_Siphoviridae.g_Corndoglikevirus
k_Viruses.f_Tombusviridae.g_Carmovirus
k_Viruses.f_Parvoviridae.g_Ambidensovirus
k_Viruses.g_Sobemovirus
k_Viruses.o_Picornavirales.f_Secoviridae.g_Comovirus
k_Viruses.o_Mononegavirales.f_Filoviridae.g_Ebolavirus
k_Viruses.f_Geminiviridae.g_Mastrevirus
k_Viruses.g_Polemovirus
k_Viruses.o_Picornavirales.f_Picornaviridae.g_Passerivirus
k_Viruses.f_Leviviridae.g_Levivirus
k_Viruses.o_Picornavirales.f_Picornaviridae.g_Rosavirus
k_Viruses.f_Retroviridae.g_Deltaretrovirus
k_Viruses.o_Mononegavirales.f_Paramyxoviridae.g_Aquaparamyxovirus
k_Viruses.f_Parvoviridae.g_Hepandensovirus
k_Viruses.o_Caudovirales.f_Podoviridae.g_Phieco32likevirus
k_Viruses.f_Reoviridae.g_Aquareovirus
k_Viruses.f_Bromoviridae.g_Ilarvirus
k_Viruses.f_Nanoviridae.g_Nanovirus
k_Viruses.f_Totiviridae.g_Trichomonasvirus
k_Viruses.f_Nanoviridae.g_Babuvirus
k_Viruses.f_Papillomaviridae.g_Dyoomikronpapillomavirus
k_Viruses.f_Caulimoviridae.g_Soymovirus
k_Viruses.f_Tombusviridae.g_Tombusvirus
k_Viruses.f_Totiviridae.g_Leishmaniavirus
k_Viruses.o_Caudovirales.f_Siphoviridae.g_Phic3unalikevirus
k_Viruses.f_Parvoviridae.g_Bocaparvovirus
k_Viruses.o_Caudovirales.f_Siphoviridae.g_Hk578likevirus
k_Viruses.o_Caudovirales.f_Siphoviridae.g_Tm4likevirus
k_Viruses.o_Caudovirales.f_Siphoviridae.g_Che8likevirus
k_Viruses.f_Tombusviridae.g_Panicovirus
k_Viruses.f_Papillomaviridae.g_Nupapillomavirus
k_Viruses.f_Orthomyxoviridae.g_Influenzavirus_A
k_Viruses.o_Caudovirales.f_Podoviridae.g_Ahjdlikevirus
k_Viruses.o_Caudovirales.f_Podoviridae.g_Luz24likevirus
k_Viruses.f_Anelloviridae.g_Zetatorquevirus
k_Viruses.f_Barnaviridae.g_Barnavirus
k_Viruses.f_Parvoviridae.g_Tetraparvovirus
k_Viruses.f_Narnaviridae.g_Mitovirus
k_Viruses.o_Caudovirales.f_Siphoviridae.g_Charlielikevirus
k_Viruses.o_Nidovirales.f_Arteriviridae.g_Arterivirus
k_Viruses.o_Mononegavirales.f_Rhabdoviridae.g_Perhabdovirus
k_Viruses.f_Potyviridae.g_Ipomovirus
k_Viruses.f_Geminiviridae.g_Curtovirus
k_Viruses.o_Picornavirales.f_Picornaviridae.g_Gallivirus
k_Viruses.o_Caudovirales.f_Siphoviridae.g_Iebhlikevirus
k_Viruses.o_Caudovirales.f_Myoviridae.g_Mulikevirus
k_Viruses.o_Picornavirales.g_Bacillarnavirus
k_Viruses.f_Megabirnaviridae.g_Megabirnavirus
k_Viruses.f_Corticoviridae.g_Corticovirus
k_Viruses.f_Bunyaviridae.g_Nairovirus
k_Viruses.f_Caulimoviridae.g_Tungrovirus
k_Viruses.f_Retroviridae.g_Spumavirus
k_Viruses.f_Reoviridae.g_Mycoreovirus
k_Viruses.o_Caudovirales.f_Myoviridae.g_Bcep78likevirus
k_Viruses.g_Idaeovirus
k_Viruses.f_Narnaviridae.g_Narnavirus
k_Viruses.f_Reoviridae.g_Coltivirus
k_Viruses.f_Totiviridae.g_Totivirus
k_Viroids.f_Pospiviroidae.g_Cocadviroid
k_Viruses.o_Caudovirales.f_Siphoviridae.g_Chilikevirus
k_Viruses.f_Potyviridae.g_Tritimovirus
k_Viruses.f_Papillomaviridae.g_Kappapapillomavirus
k_Viruses.f_Reoviridae.g_Dinovernavirus
k_Viruses.f_Anelloviridae.g_Kappatorquevirus
k_Viruses.f_Potyviridae.g_Poacevirus
k_Viruses.f_Iridoviridae.g_Megalocytivirus
k_Viruses.o_Mononegavirales.f_Rhabdoviridae.g_Nucleorhabdovirus
k_Viruses.o_Mononegavirales.f_Rhabdoviridae.g_Tibrovirus
k_Viruses.f_Closteroviridae.g_Velarivirus
k_Viruses.f_Papillomaviridae.g_Rhopapillomavirus
k_Viruses.o_Tymovirales.f_Betaflexiviridae.g_Capillovirus
k_Viruses.g_Varicosavirus
k_Viruses.f_Hepadnaviridae.g_Avihepadnavirus
k_Viruses.o_Caudovirales.f_Siphoviridae.g_Bronlikevirus
k_Viroids.f_Pospiviroidae.g_Coleviroid
k_Viroids.f_Avsunviroidae.g_Pelamoviroid
k_Viruses.o_Caudovirales.f_Podoviridae.g_Bppunalikevirus
k_Viruses.o_Caudovirales.f_Siphoviridae.g_D3likevirus
k_Viruses.f_Caliciviridae.g_Norovirus
k_Viruses.f_Leviviridae.g_Allolevivirus
k_Viruses.f_Microviridae.g_Bdellomicrovirus
k_Viruses.o_Caudovirales.f_Siphoviridae.g_Omegalikevirus
k_Viruses.o_Picornavirales.f_Picornaviridae.g_Sakobuvirus
k_Viruses.f_Plasmaviridae.g_Plasmavirus
k_Viroids.f_Pospiviroidae.g_Apscaviroid
k_Viruses.o_Caudovirales.f_Siphoviridae.g_Pbiunalikevirus
k_Viruses.f_Microviridae.g_Spiromicrovirus
k_Viruses.f_Papillomaviridae.g_Dyopipapillomavirus
k_Viruses.o_Caudovirales.f_Siphoviridae.g_Cjwunalikevirus
k_Bacteria.p_Proteobacteria.c_Gammaproteobacteria.o_Oceanospirillales.f_Halomonadaceae.g_Cobetia
k_Bacteria.p_Firmicutes.c_Clostridia.o_Thermoanaerobacterales.f_Thermoanaerobacteraceae.g_Ammonifex
k_Bacteria.p_Actinobacteria.c_Actinobacteria.o_Corynebacteriales.f_Tsukamurellaceae.g_Tsukamurella
k_Bacteria.p_Bacteroidetes.c_Flavobacteriia.o_Flavobacteriales.f_Cryomorphaceae.g_Owenweeksia
k_Bacteria.o_Haloplasmatales.f_Haloplasmataceae.g_Haloplasma
k_Bacteria.p_Proteobacteria.c_Deltaproteobacteria.o_Desulfobacterales.f_Desulfobacteraceae.g_Desulfosarcina
k_Bacteria.p_Proteobacteria.c_Gammaproteobacteria.o_Enterobacteriales.f_Enterobacteriaceae.g_Siccibacter
k_Bacteria.p_Proteobacteria.c_Alphaproteobacteria.o_Rhodospirillales.f_Acetobacteraceae.g_Asaia
k_Bacteria.p_Proteobacteria.c_Alphaproteobacteria.o_Rhizobiales.f_Rhodobiaceae.g_Parvibaculum
k_Bacteria.p_Cyanobacteria.o_Chroococcales.g_Neosynechococcus
k_Bacteria.p_Proteobacteria.c_Alphaproteobacteria.o_Sphingomonadales.f_Sphingomonadaceae.g_Zymomonas
k_Bacteria.p_Proteobacteria.c_Betaproteobacteria.o_Rhodocyclales.f_Rhodocyclaceae.g_Sulfuritalea
k_Bacteria.p_Actinobacteria.c_Actinobacteria.o_Pseudonocardiales.f_Pseudonocardiaceae.g_Kutzneria
k_Bacteria.p_Proteobacteria.c_Gammaproteobacteria.o_Chromatiales.f_Chromatiaceae.g_Marichromatium
k_Bacteria.p_Proteobacteria.c_Alphaproteobacteria.o_Rhizobiales.g_Methyloceanibacter
k_Bacteria.p_Firmicutes.c_Bacilli.o_Bacillales.f_Bacillaceae.g_Anoxybacillus
k_Bacteria.p_Proteobacteria.c_Alphaproteobacteria.o_Rhodobacterales.f_Rhodobacteraceae.g_Roseivivax
k_Archaea.p_Euryarchaeota.c_Halobacteria.o_Natrialbales.f_Natrialbaceae.g_Haloterrigena
k_Archaea.p_Euryarchaeota.c_Halobacteria.o_Natrialbales.f_Natrialbaceae.g_Halobiforma
k_Bacteria.p_Proteobacteria.c_Gammaproteobacteria.o_Enterobacteriales.f_Enterobacteriaceae.g_Pragia
k_Bacteria.p_Firmicutes.c_Clostridia.o_Clostridiales.f_Clostridiaceae.g_Fervidicella
k_Bacteria.p_Chloroflexi.c_Anaerolineae.o_Anaerolineales.f_Anaerolineaceae.g_Ornatilinea
k_Bacteria.p_Proteobacteria.c_Gammaproteobacteria.o_Enterobacteriales.f_Enterobacteriaceae.g_Wigglesworthia
k_Bacteria.p_Cyanobacteria.o_Nostocales.f_Nostocaceae.g_Dolichospermum
k_Bacteria.p_Proteobacteria.c_Deltaproteobacteria.o_Desulfarculales.f_Desulfarculaceae.g_Desulfocarbo
k_Bacteria.p_Bacteroidetes.c_Bacteroidia.o_Bacteroidales.f_Porphyromonadaceae.g_Barnesiella
k_Bacteria.p_Proteobacteria.c_Alphaproteobacteria.o_Rhizobiales.f_Phyllobacteriaceae.g_Aliihoeflea
k_Bacteria.p_Gemmatimonadetes.c_Gemmatimonadetes.o_Gemmatimonadales.f_Gemmatimonadaceae.g_Gemmatimonas
k_Bacteria.p_Proteobacteria.c_Gammaproteobacteria.o_Pasteurellales.f_Pasteurellaceae.g_Actinobacillus
k_Bacteria.p_Proteobacteria.c_Gammaproteobacteria.o_Chromatiales.f_Ectothiorhodospiraceae.g_Ectothiorhodospira
k_Bacteria.p_Proteobacteria.c_Gammaproteobacteria.o_Enterobacteriales.f_Enterobacteriaceae.g_Proteus
k_Bacteria.p_Firmicutes.c_Clostridia.o_Clostridiales.f_Eubacteriaceae.g_Eubacterium
k_Bacteria.p_Proteobacteria.c_Betaproteobacteria.o_Burkholderiales.f_Comamonadaceae.g_Polaromonas
k_Bacteria.p_Cyanobacteria.o_Chroococcales.g_Cyanobacterium
k_Bacteria.p_Proteobacteria.c_Gammaproteobacteria.o_Enterobacteriales.f_Enterobacteriaceae.g_Cronobacter
k_Bacteria.p_Bacteroidetes.c_Flavobacteriia.o_Flavobacteriales.f_Flavobacteriaceae.g_Leeuwenhoekiella
k_Bacteria.p_Actinobacteria.c_Actinobacteria.o_Micrococcales.f_Dermabacteraceae.g_Dermabacter
k_Bacteria.p_Proteobacteria.c_Betaproteobacteria.o_Rhodocyclales.f_Rhodocyclaceae.g_Azoarcus
k_Bacteria.p_Bacteroidetes.c_Chitinophagia.o_Chitinophagales.f_Saprospiraceae.g_Phaeodactylibacter
k_Bacteria.p_Proteobacteria.c_Betaproteobacteria.o_Burkholderiales.f_Oxalobacteraceae.g_Duganella
k_Bacteria.p_Proteobacteria.c_Gammaproteobacteria.o_Enterobacteriales.f_Enterobacteriaceae.g_Tatumella
k_Bacteria.p_Aquificae.c_Aquificae.o_Aquificales.f_Aquificaceae.g_Hydrogenivirga
k_Bacteria.p_Firmicutes.c_Bacilli.o_Bacillales.f_Bacillaceae.g_Thalassobacillus
k_Bacteria.p_Firmicutes.c_Clostridia.o_Clostridiales.f_Clostridiaceae.g_Candidatus_Arthromitus
k_Bacteria.p_Proteobacteria.c_Gammaproteobacteria.o_Oceanospirillales.f_Oceanospirillaceae.g_Neptunomonas
k_Bacteria.p_Proteobacteria.c_Gammaproteobacteria.o_Alteromonadales.f_Alteromonadaceae.g_Salinimonas
k_Bacteria.p_Bacteroidetes.c_Flavobacteriia.o_Flavobacteriales.f_Flavobacteriaceae.g_Weeksella
k_Bacteria.p_Bacteroidetes.c_Cytophagia.o_Cytophagales.f_Cytophagaceae.g_Runella
k_Bacteria.p_Actinobacteria.c_Actinobacteria.o_Micrococcales.f_Micrococcaceae.g_Enteractinococcus
k_Bacteria.p_Actinobacteria.c_Actinobacteria.o_Micrococcales.f_Ruaniaceae.g_Ruania
k_Bacteria.p_Proteobacteria.c_Betaproteobacteria.o_Burkholderiales.f_Burkholderiaceae.g_Pandoraea
k_Bacteria.p_Acidobacteria.c_Holophagae.o_Holophagales.f_Holophagaceae.g_Geothrix
k_Bacteria.p_Firmicutes.c_Clostridia.o_Thermoanaerobacterales.f_Thermoanaerobacterales_Family_III._Incertae_Sedis.
g_Thermoanaerobacterium
k_Bacteria.p_Firmicutes.c_Negativicutes.o_Selenomonadales.f_Sporomusaceae.g_Anaeroarcus
k_Bacteria.p_Actinobacteria.c_Actinobacteria.o_Micrococcales.f_Demequinaceae.g_Lysinimicrobium
k_Bacteria.p_Proteobacteria.c_Gammaproteobacteria.o_Pasteurellales.f_Pasteurellaceae.g_Bibersteinia
k_Bacteria.p_Firmicutes.c_Clostridia.o_Clostridiales.f_Lachnospiraceae.g_Dorea
k_Bacteria.p_Actinobacteria.c_Actinobacteria.o_Micrococcales.f_Microbacteriaceae.g_Cryocola
k_Bacteria.p_Firmicutes.c_Clostridia.o_Clostridiales.f_Peptococcaceae.g_Candidatus_Desulforudis
k_Bacteria.p_Thermotogae.c_Thermotogae.o_Petrotogales.g_Defluviitoga
k_Bacteria.p_Firmicutes.c_Clostridia.o_Clostridiales.f_Peptococcaceae.g_Desulfitobacterium
k_Bacteria.p_Actinobacteria.c_Actinobacteria.o_Streptosporangiales.f_Streptosporangiaceae.g_Streptosporangium
k_Bacteria.p_Chlamydiae.c_Chlamydiia.o_Chlamydiales.f_Parachlamydiaceae.g_Neochlamydia
k_Bacteria.p_Bacteroidetes.c_Flavobacteriia.o_Flavobacteriales.f_Flavobacteriaceae.g_Croceibacter
k_Bacteria.p_Deinococcus.Thermus.c_Deinococci.o_Deinococcales.f_Trueperaceae.g_Truepera
k_Bacteria.p_Proteobacteria.c_Alphaproteobacteria.o_Rhodospirillales.f_Acetobacteraceae.g_Kozakia
k_Bacteria.p_Proteobacteria.c_Alphaproteobacteria.o_Rhodobacterales.f_Hyphomonadaceae.g_Ponticaulis
k_Bacteria.p_Firmicutes.c_Bacilli.o_Bacillales.f_Thermoactinomycetaceae.g_Risungbinella
k_Bacteria.p_Cyanobacteria.o_Oscillatoriales.g_Arthrospira
k_Bacteria.p_Proteobacteria.c_Gammaproteobacteria.o_Legionellales.f_Coxiellaceae.g_Rickettsiella
k_Bacteria.p_Firmicutes.c_Bacilli.o_Lactobacillales.f_Carnobacteriaceae.g_Carnobacterium
k_Bacteria.p_Proteobacteria.c_Gammaproteobacteria.o_Orbales.f_Orbaceae.g_Gilliamella
k_Bacteria.p_Proteobacteria.c_Betaproteobacteria.o_Neisseriales.f_Chromobacteriaceae.g_Laribacter
k_Bacteria.p_Proteobacteria.c_Gammaproteobacteria.o_Vibrionales.f_Vibrionaceae.g_Saltnivibrio
k_Bacteria.p_Bacteroidetes.c_Flavobacteriia.o_Flavobacteriales.f_Flavobacteriaceae.g_Formosa
k_Bacteria.p_Proteobacteria.c_Gammaproteobacteria.o_Enterobacteriales.f_Enterobacteriaceae.g_Cedecea
k_Bacteria.p_Actinobacteria.c_Actinobacteria.o_Acidothermales.f_Acidothermaceae.g_Acidothermus
k_Bacteria.p_Actinobacteria.c_Actinobacteria.o_Micromonosporales.f_Micromonosporaceae.g_Micromonospora
k_Bacteria.p_Cyanobacteria.o_Pleurocapsales.g_Stanieria
k_Bacteria.p_Proteobacteria.c_Deltaproteobacteria.o_Myxococcales.f_Cystobacteraceae.g_Hyalangium
k_Bacteria.p_Bacteroidetes.c_Flavobacteriia.o_Flavobacteriales.f_Flavobacteriaceae.g_Imtechella
k_Bacteria.p_Firmicutes.c_Bacilli.o_Bacillales.f_Bacillaceae.g_Caldibacillus
k_Bacteria.p_Proteobacteria.c_Gammaproteobacteria.o_Enterobacteriales.f_Enterobacteriaceae.g_Hafnia
k_Bacteria.p_Proteobacteria.c_Gammaproteobacteria.o_Enterobacteriales.f_Enterobacteriaceae.g_Yersinia
k_Bacteria.p_Firmicutes.c_Clostridia.o_Clostridiales.f_Eubacteriaceae.g_Acetobacterium
k_Bacteria.p_Actinobacteria.c_Actinobacteria.o_Corynebacteriales.f_Nocardiaceae.g_Nocardia
k_Bacteria.p_Firmicutes.c_Bacilli.o_Lactobacillales.f_Aerococcaceae.g_Facklamia
k_Bacteria.p_Proteobacteria.c_Betaproteobacteria.o_Rhodocyclales.f_Rhodocyclaceae.g_Aromatoleum
k_Bacteria.p_Firmicutes.c_Clostridia.o_Clostridiales.f_Peptostreptococcaceae.g_Clostridioides
k_Bacteria.p_Acidobacteria.c_Acidobacteriia.o_Acidobacteriales.f_Acidobacteriaceae.g_Terracidiphilus
k_Bacteria.p_Proteobacteria.c_Gammaproteobacteria.o_Xanthomonadales.f_Xanthomonadaceae.g_Thermomonas
k_Bacteria.p_Cyanobacteria.o_Chroococcales.g_Crocosphaera
k_Bacteria.p_Actinobacteria.c_Actinobacteria.o_Jiangellales.f_Jiangellaceae.g_Jiangella
k_Bacteria.p_Proteobacteria.c_Alphaproteobacteria.o_Rhodospirillales.f_Acetobacteraceae.g_Roseomonas
k_Bacteria.p_Synergistetes.c_Synergistia.o_Synergistales.f_Synergistaceae.g_Aminiphilus
k_Bacteria.p_Bacteroidetes.c_Flavobacteriia.o_Flavobacteriales.f_Flavobacteriaceae.g_Lacinutrix
k_Bacteria.p_Actinobacteria.c_Actinobacteria.o_Actinomycetales.f_Actinomycetaceae.g_Mobiluncus
k_Bacteria.p_Thermotogae.c_Thermotogae.o_Petrotogales.g_Marinitoga
k_Bacteria.p_Proteobacteria.c_Gammaproteobacteria.o_Enterobacteriales.f_Enterobacteriaceae.g_Rouxiella
k_Bacteria.p_Cyanobacteria.o_Chroococcales.g_Thermosynechococcus
k_Bacteria.p_Proteobacteria.c_Alphaproteobacteria.o_Rhodobacterales.f_Rhodobacteraceae.g_Tropicibacter
k_Bacteria.p_Firmicutes.c_Clostridia.o_Clostridiales.f_Syntrophomonadaceae.g_Syntrophothermus
k_Archaea.p_Crenarchaeota.c_Thermoprotei.o_Desulfurococcales.f_Pyrodictiaceae.g_Hyperthermus
k_Bacteria.p_Planctomycetes.c_Planctomycetia.o_Planctomycetales.f_Planctomycetaceae.g_Pirellula
k_Bacteria.p_Proteobacteria.c_Alphaproteobacteria.o_Rhodobacterales.f_Rhodobacteraceae.g_Maritimibacter
k_Bacteria.p_Firmicutes.c_Bacilli.o_Bacillales.f_Bacillaceae.g_Salsuginibacillus
k_Bacteria.p_Synergistetes.c_Synergistia.o_Synergistales.f_Synergistaceae.g_Thermanaerovibrio
k_Bacteria.p_Proteobacteria.c_Gammaproteobacteria.o_Cardiobacteriales.f_Cardiobacteriaceae.g_Dichelobacter
k_Bacteria.p_Actinobacteria.c_Actinobacteria.o_Bifidobacteriales.f_Bifidobacteriaceae.g_Bifidobacterium
k_Bacteria.p_Actinobacteria.c_Coriobacteriia.o_Coriobacteriales.f_Coriobacteriaceae.g_Collinsella
k_Bacteria.p_Proteobacteria.c_Alphaproteobacteria.o_Caulobacterales.f_Caulobacteraceae.g_Brevundimonas
k_Bacteria.p_Proteobacteria.c_Gammaproteobacteria.o_Alteromonadales.f_Alteromonadaceae.g_Aliiglaciecola
k_Bacteria.p_Bacteroidetes.c_Cytophagia.o_Cytophagales.f_Cytophagaceae.g_Anditalea
k_Bacteria.p_Actinobacteria.c_Actinobacteria.o_Micrococcales.f_Microbacteriaceae.g_Clavibacter
k_Bacteria.p_Proteobacteria.c_Gammaproteobacteria.o_Nevskiales.f_Sinobacteraceae.g_Polycyclovorans
k_Bacteria.p_Bacteroidetes.c_Flavobacteriia.o_Flavobacteriales.f_Flavobacteriaceae.g_Epilithonimonas
k_Bacteria.p_Actinobacteria.c_Actinobacteria.o_Bifidobacteriales.f_Bifidobacteriaceae.g_Gardnerella
k_Bacteria.p_Actinobacteria.c_Actinobacteria.o_Corynebacteriales.f_Corynebacteriaceae.g_Turicella
k_Bacteria.p_Proteobacteria.c_Alphaproteobacteria.o_Rhodobacterales.f_Rhodobacteraceae.g_Oceanicola
k_Bacteria.p_Deinococcus.Thermus.c_Deinococci.o_Thermales.f_Thermaceae.g_Meiothermus
k_Bacteria.p_Firmicutes.c_Negativicutes.o_Acidaminococcales.f_Acidaminococcaceae.g_Phascolarctobacterium
k_Bacteria.p_Bacteroidetes.c_Sphingobacteriia.o_Sphingobacteriales.f_Sphingobacteriaceae.g_Arcticibacter
k_Bacteria.p_Bacteroidetes.c_Cytophagia.o_Cytophagales.f_Cyclobacteriaceae.g_Echinicola
k_Bacteria.p_Actinobacteria.c_Coriobacteriia.o_Eggerthellales.f_Eggerthellaceae.g_Eggerthella
k_Bacteria.p_Actinobacteria.c_Actinobacteria.o_Micrococcales.f_Micrococcaceae.g_Pseudoglutamicibacter
k_Bacteria.p_Bacteroidetes.c_Flavobacteriia.o_Flavobacteriales.f_Flavobacteriaceae.g_Altibacter
k_Bacteria.p_Proteobacteria.c_Deltaproteobacteria.o_Myxococcales.f_Labilitrichaceae.g_Labilithrix
k_Bacteria.p_Firmicutes.c_Clostridia.o_Clostridiales.f_Catabacteriaceae.g_Catabacter
k_Archaea.p_Euryarchaeota.c_Thermoplasmata.o_Methanomassiliicoccales.f_Methanomassiliicoccaceae.g_Candidatus_Methanomethylophilus
k_Archaea.p_Euryarchaeota.c_Halobacteria.o_Halobacteriales.f_Halobacteriaceae.g_Haloarcula
k_Archaea.p_Euryarchaeota.c_Methanobacteria.o_Methanobacteriales.f_Methanobacteriaceae.g_Methanothermobacter
k_Bacteria.p_Bacteroidetes.c_Cytophagia.o_Cytophagales.f_Cytophagaceae.g_Fibrisoma
k_Bacteria.p_Bacteroidetes.c_Flavobacteriia.o_Flavobacteriales.g_Candidatus_Sulcia
k_Bacteria.p_Firmicutes.c_Clostridia.o_Clostridiales.f_Peptococcaceae.g_Desulfosporosinus
k_Bacteria.p_Proteobacteria.c_Betaproteobacteria.o_Burkholderiales.f_Burkholderiaceae.g_Caballeronia
k_Bacteria.p_Fibrobacteres.c_Chitinispirillia.o_Chitinispirillales.f_Chitinispirillaceae.g_Chitinispirillum
k_Bacteria.p_Actinobacteria.c_Actinobacteria.o_Micrococcales.f_Micrococcaceae.g_Sinomonas
k_Bacteria.p_Proteobacteria.c_Alphaproteobacteria.o_Rhodobacterales.f_Rhodobacteraceae.g_Ruegeria
k_Bacteria.p_Proteobacteria.c_Gammaproteobacteria.o_Alteromonadales.f_Shewanellaceae.g_Shewanella
k_Bacteria.p_Proteobacteria.c_Gammaproteobacteria.o_Enterobacteriales.f_Enterobacteriaceae.g_Serratia
k_Bacteria.p_Proteobacteria.c_Betaproteobacteria.o_Burkholderiales.g_Tepidimonas
k_Bacteria.p_Firmicutes.c_Negativicutes.g_Anaerosporomusa
k_Bacteria.p_Proteobacteria.c_Gammaproteobacteria.o_Alteromonadales.f_Pseudoalteromonadaceae.g_Pseudoalteromonas
k_Bacteria.p_Bacteroidetes.c_Cytophagia.o_Cytophagales.f_Cyclobacteriaceae.g_Nitritalea
k_Bacteria.p_Firmicutes.c_Negativicutes.o_Veillonellales.f_Veillonellaceae.g_Veillonella
k_Bacteria.p_Firmicutes.c_Bacilli.o_Bacillales.f_Bacillaceae.g_Gracilibacillus
k_Bacteria.p_Proteobacteria.c_Gammaproteobacteria.o_Chromatiales.f_Ectothiorhodospiraceae.g_Nitrococcus
k_Bacteria.p_Proteobacteria.c_Alphaproteobacteria.o_Rhizobiales.f_Bradyrhizobiaceae.g_Rhodopseudomonas
k_Bacteria.p_Proteobacteria.c_Gammaproteobacteria.o_Legionellales.f_Coxiellaceae.g_Diplorickettsia
k_Bacteria.p_Proteobacteria.c_Deltaproteobacteria.o_Desulfuromonadales.f_Desulfuromonadaceae.g_Desulfuromonas
k_Archaea.p_Euryarchaeota.c_Methanomicrobia.o_Methanocellales.f_Methanocellaceae.g_Methanocella
k_Bacteria.p_Tenericutes.c_Mollicutes.o_Entomoplasmatales.f_Spiroplasmataceae.g_Spiroplasma
k_Bacteria.p_Proteobacteria.c_Epsilonproteobacteria.g_Sulfurovum
k_Bacteria.p_Proteobacteria.c_Gammaproteobacteria.o_Enterobacteriales.f_Enterobacteriaceae.g_Candidatus_Tachikawaea
k_Bacteria.p_Proteobacteria.c_Alphaproteobacteria.o_Rhizobiales.f_Bradyrhizobiaceae.g_Oligotropha
k_Bacteria.p_Proteobacteria.c_Gammaproteobacteria.o_Enterobacteriales.f_Enterobacteriaceae.g_Mangrovibacter
k_Bacteria.p_Proteobacteria.c_Deltaproteobacteria.o_Desulfovibrionales.f_Desulfonatronaceae.g_Desulfonatronum
k_Bacteria.p_Cyanobacteria.o_Oscillatoriales.g_Moorea
k_Bacteria.p_Proteobacteria.c_Alphaproteobacteria.o_Rhodospirillales.f_Rhodospirillaceae.g_Pararhodospirillum
k_Bacteria.p_Proteobacteria.c_Betaproteobacteria.o_Burkholderiales.f_Burkholderiaceae.g_Burkholderia
k_Bacteria.p_Bacteroidetes.c_Cytophagia.o_Cytophagales.f_Flammeovirgaceae.g_Cesiribacter
k_Archaea.p_Euryarchaeota.c_Methanomicrobia.o_Methanomicrobiales.f_Methanocorpusculaceae.g_Methanocorpusculum
k_Bacteria.p_Tenericutes.c_Mollicutes.o_Acholeplasmatales.f_Acholeplasmataceae.g_Candidatus_Phytoplasma
k_Archaea.p_Euryarchaeota.c_Halobacteria.o_Natrialbales.f_Natrialbaceae.g_Natronolimnobius
k_Bacteria.p_Firmicutes.c_Bacilli.o_Bacillales.f_Bacillaceae.g_Caldalkalibacillus
k_Bacteria.p_Proteobacteria.c_Gammaproteobacteria.g_Ignatzschineria
k_Bacteria.p_Proteobacteria.c_Gammaproteobacteria.o_Thiotrichales.f_Thiotrichaceae.g_Beggiatoa
k_Bacteria.p_Actinobacteria.c_Actinobacteria.o_Kineosporiales.f_Kineosporiaceae.g_Kineococcus
k_Bacteria.p_Bacteroidetes.c_Flavobacteriia.o_Flavobacteriales.f_Flavobacteriaceae.g_Aquimarina
k_Archaea.p_Crenarchaeota.c_Thermoprotei.o_Desulfurococcales.f_Desulfurococcaceae.g_Staphylothermus
k_Archaea.p_Crenarchaeota.c_Thermoprotei.o_Acidilobales.f_Caldisphaeraceae.g_Caldisphaera
k_Bacteria.p_Actinobacteria.c_Actinobacteria.o_Micrococcales.f_Microbacteriaceae.g_Frondihabitans
k_Bacteria.p_Actinobacteria.c_Actinobacteria.o_Micromonosporales.f_Micromonosporaceae.g_Salinispora
k_Bacteria.p_Actinobacteria.c_Actinobacteria.o_Micrococcales.f_Micrococcaceae.g_Pseudarthrobacter
k_Bacteria.p_Proteobacteria.c_Betaproteobacteria.o_Burkholderiales.f_Burkholderiaceae.g_Paraburkholderia
k_Archaea.p_Euryarchaeota.c_Methanobacteria.o_Methanobacteriales.f_Methanobacteriaceae.g_Methanosphaera
k_Bacteria.p_Proteobacteria.c_Gammaproteobacteria.o_Alteromonadales.f_Colwelliaceae.g_Thalassotalea
k_Bacteria.p_Proteobacteria.c_Gammaproteobacteria.o_Enterobacteriales.f_Enterobacteriaceae.g_Morganella
k_Bacteria.p_Firmicutes.c_Erysipelotrichia.o_Erysipelotrichales.f_Erysipelotrichaceae.g_Erysipelothrix
k_Bacteria.p_Spirochaetes.c_Spirochaetia.o_Spirochaetales.f_Borreliaceae.g_Borrelia
k_Bacteria.p_Planctomycetes.c_Planctomycetia.o_Planctomycetales.f_Isosphaeraceae.g_Isosphaera
k_Bacteria.p_Proteobacteria.c_Alphaproteobacteria.o_Rhodobacterales.f_Rhodobacteraceae.g_Shimia
k_Bacteria.p_Firmicutes.c_Bacilli.o_Bacillales.f_Planococcaceae.g_Planococcus
k_Bacteria.p_Firmicutes.c_Bacilli.o_Bacillales.g_Acidibacillus
k_Bacteria.p_Proteobacteria.c_Gammaproteobacteria.o_Enterobacteriales.f_Enterobacteriaceae.g_Kluyvera
k_Bacteria.p_Proteobacteria.c_Betaproteobacteria.o_Neisseriales.f_Chromobacteriaceae.g_Aquitalea
k_Bacteria.p_Firmicutes.c_Clostridia.o_Clostridiales.f_Syntrophomonadaceae.g_Dethiobacter
k_Bacteria.p_Bacteroidetes.c_Bacteroidia.o_Bacteroidales.f_Rikenellaceae.g_Rikenella
k_Bacteria.p_Firmicutes.c_Erysipelotrichia.o_Erysipelotrichales.f_Erysipelotrichaceae.g_Erysipelatoclostridium
k_Bacteria.p_Cyanobacteria.o_Nostocales.f_Rivulariaceae.g_Calothrix
k_Bacteria.p_Proteobacteria.c_Betaproteobacteria.o_Rhodocyclales.f_Rhodocyclaceae.g_Uliginosibacterium
k_Bacteria.p_Actinobacteria.c_Actinobacteria.o_Propionibacteriales.f_Nocardioidaceae.g_Marmoricola
k_Bacteria.p_Bacteroidetes.c_Cytophagia.o_Cytophagales.f_Flammeovirgaceae.g_Fulvivirga
k_Bacteria.p_Chloroflexi.c_Chloroflexia.o_Chloroflexales.f_Roseiflexaceae.g_RoseiflexuS
k_Bacteria.p_Proteobacteria.c_Betaproteobacteria.o_Neisseriales.f_Chromobacteriaceae.g_Vogesella
k_Bacteria.p_Proteobacteria.c_Alphaproteobacteria.o_Rhodobacterales.f_Rhodobacteraceae.g_Falsirhodobacter
k_Bacteria.p_Firmicutes.c_Clostridia.o_Clostridiales.f_Eubacteriaceae.g_Pseudoramibacter
k_Bacteria.p_Proteobacteria.c_Gammaproteobacteria.o_Oceanospirillales.f_Saccharospirillaceae.g_Reinekea
k_Bacteria.p_Proteobacteria.c_Alphaproteobacteria.o_Rhodobacterales.f_Hyphomonadaceae.g_Robiginitomaculum
k_Bacteria.p_Firmicutes.c_Clostridia.o_Clostridiales.f_Peptostreptococcaceae.g_Peptoclostridium
k_Bacteria.p_Tenericutes.c_Mollicutes.o_Entomoplasmatales.f_Entomoplasmataceae.g_Mesoplasma
k_Bacteria.p_Proteobacteria.c_Epsilonproteobacteria.o_Nautiliales.f_Nautiliaceae.g_Caminibacter
k_Bacteria.p_Proteobacteria.c_Alphaproteobacteria.o_Rhizobiales.f_Rhizobiaceae.g_Sinorhizobium
k_Bacteria.p_Verrucomicrobia.c_Verrucomicrobiae.o_Verrucomicrobiales.f_Akkermansiaceae.g_Akkermansia
k_Bacteria.p_Firmicutes.c_Tissierellia.o_Tissierellales.f_Peptoniphilaceae.g_Finegoldia
k_Archaea.p_Euryarchaeota.c_Halobacteria.o_Halobacteriales.f_Halobacteriaceae.g_Natronomonas
k_Bacteria.p_Proteobacteria.c_Betaproteobacteria.o_Burkholderiales.f_Oxalobacteraceae.g_Massilia
k_Bacteria.p_Proteobacteria.c_Alphaproteobacteria.o_Rhizobiales.f_Aurantimonadaceae.g_Fulvimarina
k_Bacteria.p_Bacteroidetes.c_Cytophagia.o_Cytophagales.f_Amoebophilaceae.g_Candidatus_Amoebophilus
k_Bacteria.p_Tenericutes.c_Mollicutes.o_Acholeplasmatales.f_Acholeplasmataceae.g_Acholeplasma
k_Bacteria.p_Proteobacteria.c_Alphaproteobacteria.o_Rhizobiales.f_Phyllobacteriaceae.g_Pseudaminobacter
k_Bacteria.p_Proteobacteria.c_Gammaproteobacteria.o_Enterobacteriales.f_Enterobacteriaceae.g_Phaseolibacter
k_Bacteria.p_Bacteroidetes.c_Flavobacteriia.o_Flavobacteriales.f_Flavobacteriaceae.g_Salinimicrobium
k_Bacteria.p_Bacteroidetes.c_Bacteroidia.o_Bacteroidales.f_Prolixibacteraceae.g_Draconibacterium
k_Bacteria.p_Firmicutes.c_Bacilli.o_Lactobacillales.f_Aerococcaceae.g_Abiotrophia
k_Bacteria.p_Proteobacteria.c_Gammaproteobacteria.o_Enterobacteriales.f_Enterobacteriaceae.g_Dickeya
k_Bacteria.p_Proteobacteria.c_Alphaproteobacteria.o_Rhodospirillales.g_Reyranella
k_Bacteria.p_Bacteroidetes.c_Cytophagia.o_Cytophagales.f_Cyclobacteriaceae.g_Belliella
k_Bacteria.p_Proteobacteria.c_Alphaproteobacteria.o_Rhodobacterales.f_Rhodobacteraceae.g_Rhodovulum
k_Bacteria.p_Firmicutes.c_Clostridia.o_Halanaerobiales.f_Halanaerobiaceae.g_Halanaerobium
k_Bacteria.p_Actinobacteria.c_Actinobacteria.o_Micromonosporales.f_Micromonosporaceae.g_Actinocatenispora
k_Bacteria.p_Proteobacteria.c_Alphaproteobacteria.o_Rhodobacterales.f_Rhodobacteraceae.g_Aestuariivita
k_Bacteria.p_Actinobacteria.c_Actinobacteria.o_Pseudonocardiales.f_Pseudonocardiaceae.g_Actinosynnema
k_Archaea.p_Euryarchaeota.c_Halobacteria.o_Natrialbales.f_Natrialbaceae.g_Natronobacterium
k_Bacteria.p_Proteobacteria.c_Gammaproteobacteria.o_Alteromonadales.f_Alteromonadaceae.g_Aliagarivorans
k_Bacteria.p_Bacteroidetes.c_Cytophagia.o_Cytophagales.f_Cytophagaceae.g_Leadbetterella
k_Bacteria.p_Proteobacteria.c_Betaproteobacteria.o_Methylophilales.f_Methylophilaceae.g_Methylotenera
k_Bacteria.p_Nitrospirae.c_Nitrospira.o_Nitrospirales.f_Nitrospiraceae.g_Leptospirillum
k_Bacteria.p_Fusobacteria.c_Fusobacteriia.o_Fusobacteriales.f_Leptotrichiaceae.g_Sneathia
k_Bacteria.p_Proteobacteria.c_Betaproteobacteria.o_Burkholderiales.f_Burkholderiaceae.g_Limnobacter
k_Bacteria.p_Proteobacteria.c_Gammaproteobacteria.o_Enterobacteriales.f_Enterobacteriaceae.g_Providencia
k_Bacteria.p_Proteobacteria.c_Alphaproteobacteria.o_Rhizobiales.f_Hyphomicrobiaceae.g_Maritalea
k_Bacteria.p_Actinobacteria.c_Actinobacteria.o_Corynebacteriales.f_Nocardiaceae.g_Rhodococcus
k_Bacteria.p_Proteobacteria.c_Alphaproteobacteria.o_Rhodobacterales.f_Rhodobacteraceae.g_Wenxinia
k_Bacteria.p_Proteobacteria.c_Alphaproteobacteria.o_Rhizobiales.f_Phyllobacteriaceae.g_Chelativorans
k_Bacteria.p_Bacteroidetes.c_Flavobacteriia.o_Flavobacteriales.f_Schleiferiaceae.g_Schleiferia
k_Bacteria.p_Proteobacteria.c_Gammaproteobacteria.o_Enterobacteriales.f_Enterobacteriaceae.g_Pectobacterium
k_Bacteria.p_Proteobacteria.c_Alphaproteobacteria.o_Pelagibacterales.f_Pelagibacteraceae.g_Candidatus_Pelagibacter
k_Bacteria.p_Proteobacteria.c_Betaproteobacteria.o_Burkholderiales.g_Roseateles
k_Bacteria.p_Firmicutes.c_Bacilli.o_Bacillales.f_Staphylococcaceae.g_Staphylococcus
k_Bacteria.p_Proteobacteria.c_Betaproteobacteria.o_Burkholderiales.f_Comamonadaceae.g_Limnohabitans
k_Bacteria.p_Proteobacteria.c_Deltaproteobacteria.o_Desulfobacterales.f_Desulfobacteraceae.g_Desulfospira
k_Bacteria.p_Proteobacteria.c_Alphaproteobacteria.o_Rhodobacterales.f_Rhodobacteraceae.g_Maribius
k_Bacteria.p_Proteobacteria.c_Betaproteobacteria.o_Neisseriales.f_Neisseriaceae.g_Simonsiella
k_Bacteria.p_Actinobacteria.c_Actinobacteria.o_Micrococcales.f_Micrococcaceae.g_Glutamicibacter
k_Bacteria.p_Planctomycetes.c_Planctomycetia.o_Planctomycetales.f_Planctomycetaceae.g_Schlesneria
k_Bacteria.p_Firmicutes.c_Bacilli.o_Lactobacillales.f_Leuconostocaceae.g_Fructobacillus
k_Bacteria.p_Proteobacteria.c_Alphaproteobacteria.o_Rhodobacterales.f_Hyphomonadaceae.g_Oceanicaulis
k_Bacteria.p_Proteobacteria.c_Deltaproteobacteria.o_Desulfobacterales.f_Desulfobacteraceae.g_Desulfobacterium
k_Bacteria.p_Proteobacteria.c_Deltaproteobacteria.o_Desulfobacterales.f_Desulfobulbaceae.g_Desulfobulbus
k_Bacteria.p_Firmicutes.c_Bacilli.o_Lactobacillales.f_Lactobacillaceae.g_Sharpea
k_Bacteria.p_Chlamydiae.c_Chlamydiia.o_Chlamydiales.f_Parachlamydiaceae.g_Parachlamydia
k_Bacteria.p_Thermodesulfobacteria.c_Thermodesulfobacteria.o_Thermodesulfobacteriales.f_Thermodesulfobacteriaceae.g_Thermosulfurimonas
k_Bacteria.p_Proteobacteria.c_Gammaproteobacteria.o_Enterobacteriales.f_Enterobacteriaceae.g_Sodalis
k_Bacteria.p_Actinobacteria.c_Actinobacteria.o_Streptosporangiales.f_Streptosporangiaceae.g_Nonomuraea
k_Bacteria.p_Proteobacteria.c_Betaproteobacteria.o_Methylophilales.f_Methylophilaceae.g_Methylobacillus
k_Bacteria.p_Proteobacteria.c_Deltaproteobacteria.o_Desulfovibrionales.f_Desulfohalobiaceae.g_Desulfohalobium
k_Bacteria.p_Bacteroidetes.c_Cytophagia.o_Cytophagales.f_Flammeovirgaceae.g_Flammeovirga
k_Archaea.p_Euryarchaeota.c_Methanobacteria.o_Methanobacteriales.f_Methanobacteriaceae.g_Methanobrevibacter
k_Bacteria.p_Firmicutes.c_Clostridia.o_Clostridiales.f_Peptococcaceae.g_Dehalobacter
k_Bacteria.p_Firmicutes.c_Clostridia.o_Clostridiales.f_Lachnospiraceae.g_Lachnoclostridium
k_Bacteria.p_Actinobacteria.c_Actinobacteria.o_Micrococcales.f_Microbacteriaceae.g_Leucobacter
k_Bacteria.p_Cyanobacteria.o_Pleurocapsales.g_Chroococcidiopsis
k_Bacteria.p_Bacteroidetes.c_Bacteroidia.o_Bacteroidales.f_Porphyromonadaceae.g_Fermentimonas
k_Bacteria.p_Proteobacteria.c_Gammaproteobacteria.o_Xanthomonadales.f_Xanthomonadaceae.g_Silanimonas
k_Bacteria.p_Cyanobacteria.o_Nostocales.f_Nostocaceae.g_Nostoc
k_Bacteria.p_Bacteroidetes.c_Cytophagia.o_Cytophagales.f_Cytophagaceae.g_Rhodonellum
k_Bacteria.p_Firmicutes.c_Tissierellia.o_Tissierellales.f_Peptoniphilaceae.g_Anaerococcus
k_Bacteria.p_Proteobacteria.c_Gammaproteobacteria.o_Alteromonadales.f_Psychromonadaceae.g_Psychromonas
k_Bacteria.p_Bacteroidetes.c_Flavobacteriia.o_Flavobacteriales.f_Flavobacteriaceae.g_Arenitalea
k_Bacteria.p_Actinobacteria.c_Actinobacteria.o_Micromonosporales.f_Micromonosporaceae.g_Longispora
k_Bacteria.p_Actinobacteria.c_Actinobacteria.o_Propionibacteriales.f_Propionibacteriaceae.g_Granulicoccus
k_Bacteria.p_Proteobacteria.c_Gammaproteobacteria.o_Enterobacteriales.f_Enterobacteriaceae.g_Ewingella
k_Bacteria.p_Proteobacteria.c_Betaproteobacteria.o_Burkholderiales.f_Alcaligenaceae.g_Azohydromonas
k_Bacteria.p_Firmicutes.c_Bacilli.o_Lactobacillales.f_Streptococcaceae.g_Lactococcus
k_Archaea.p_Thaumarchaeota.c_Nitrososphaeria.o_Nitrososphaerales.f_Nitrososphaeraceae.g_Nitrososphaera
k_Bacteria.p_Armatimonadetes.c_Chthonomonadetes.o_Chthonomonadales.f_Chthonomonadaceae.g_Chthonomonas
k_Bacteria.p_Proteobacteria.c_Gammaproteobacteria.o_Alteromonadales.f_Ferrimonadaceae.g_Ferrimonas
k_Bacteria.p_Actinobacteria.c_Actinobacteria.o_Streptomycetales.f_Streptomycetaceae.g_Streptomyces
k_Bacteria.p_Firmicutes.c_Bacilli.o_Bacillales.f_Thermoactinomycetaceae.g_Shimazuella
k_Bacteria.p_Cyanobacteria.o_Stigonematales.g_Fischerella
k_Bacteria.p_Cyanobacteria.o_Pleurocapsales.g_Aliterella
k_Bacteria.p_Actinobacteria.c_Actinobacteria.o_Micrococcales.f_Microbacteriaceae.g_Salinibacterium
k_Bacteria.p_Proteobacteria.c_Gammaproteobacteria.o_Cellvibrionales.f_Halieaceae.g_Halioglobus
k_Bacteria.p_Proteobacteria.c_Betaproteobacteria.o_Neisseriales.f_Chromobacteriaceae.g_Chitiniphilus
k_Bacteria.p_Firmicutes.c_Bacilli.o_Bacillales.f_Planococcaceae.g_Planomicrobium
k_Bacteria.p_Proteobacteria.c_Gammaproteobacteria.o_Oceanospirillales.f_Alcanivoracaceae.g_Alcanivorax
k_Bacteria.p_Actinobacteria.c_Actinobacteria.o_Micrococcales.f_Intrasporangiaceae.g_Intrasporangium
k_Bacteria.p_Proteobacteria.c_Betaproteobacteria.o_Neisseriales.f_Neisseriaceae.g_Crenobacter
k_Bacteria.p_Firmicutes.c_Clostridia.o_Clostridiales.f_Lachnospiraceae.g_Oribacterium
k_Bacteria.p_Proteobacteria.c_Alphaproteobacteria.o_Rhodobacterales.f_Hyphomonadaceae.g_Henriciella
k_Bacteria.p_Fusobacteria.c_Fusobacteriia.o_Fusobacteriales.f_Fusobacteriaceae.g_Fusobacterium
k_Bacteria.p_Actinobacteria.c_Actinobacteria.o_Bifidobacteriales.f_Bifidobacteriaceae.g_Alloscardovia
k_Bacteria.p_Actinobacteria.c_Actinobacteria.o_Micromonosporales.f_Micromonosporaceae.g_Dactylosporangium
k_Bacteria.p_Actinobacteria.c_Actinobacteria.o_Micrococcales.f_Micrococcaceae.g_Acaricomes
k_Bacteria.p_Proteobacteria.c_Alphaproteobacteria.o_Rhodobacterales.f_Rhodobacteraceae.g_Citreicella
k_Bacteria.p_Proteobacteria.c_Betaproteobacteria.o_Burkholderiales.f_Alcaligenaceae.g_Achromobacter
k_Bacteria.p_Proteobacteria.c_Betaproteobacteria.o_Burkholderiales.f_Comamonadaceae.g_Candidatus_Symbiobacter
k_Bacteria.p_Ignavibacteriae.c_Ignavibacteria.o_Ignavibacteriales.f_Melioribacteraceae.g_Melioribacter
k_Bacteria.p_Proteobacteria.c_Alphaproteobacteria.o_Rhodobacterales.f_Rhodobacteraceae.g_Ahrensia
k_Bacteria.p_Proteobacteria.c_Gammaproteobacteria.o_Aeromonadales.f_Aeromonadaceae.g_Oceanimonas
k_Bacteria.p_Firmicutes.c_Clostridia.o_Clostridiales.g_Pseudoflavonifractor
k_Bacteria.p_Firmicutes.c_Bacilli.o_Bacillales.f_Bacillaceae.g_Domibacillus
k_Bacteria.p_Actinobacteria.c_Actinobacteria.o_Micrococcales.f_Microbacteriaceae.g_Rathayibacter
k_Bacteria.p_Proteobacteria.c_Betaproteobacteria.o_Burkholderiales.g_Rhizobacter
k_Bacteria.p_Proteobacteria.c_Gammaproteobacteria.o_Pasteurellales.f_Pasteurellaceae.g_Aggregatibacter
k_Bacteria.p_Firmicutes.c_Bacilli.o_Lactobacillales.f_Carnobacteriaceae.g_Allofustis
k_Bacteria.p_Fusobacteria.c_Fusobacteriia.o_Fusobacteriales.f_Fusobacteriaceae.g_Cetobacterium
k_Bacteria.p_Firmicutes.c_Clostridia.o_Halanaerobiales.f_Halobacteroidaceae.g_Acetohalobium
k_Bacteria.p_Proteobacteria.c_Gammaproteobacteria.o_Oceanospirillales.f_Oceanospirillaceae.g_Oceanobacter
k_Bacteria.p_Proteobacteria.c_Gammaproteobacteria.o_Legionellales.f_Legionellaceae.g_Legionella
k_Bacteria.p_Proteobacteria.c_Gammaproteobacteria.o_Cellvibrionales.f_Porticoccaceae.g_Porticoccus
k_Bacteria.p_Verrucomicrobia.c_Opitutae.o_Opitutales.f_Opitutaceae.g_Diplosphaera
k_Bacteria.p_Proteobacteria.c_Gammaproteobacteria.o_Enterobacteriales.f_Enterobacteriaceae.g_Buchnera
k_Bacteria.p_Firmicutes.c_Negativicutes.o_Acidaminococcales.f_Acidaminococcaceae.g_Succinispira
k_Bacteria.p_Chlorobi.c_Chlorobia.o_Chlorobiales.f_Chlorobiaceae.g_Chlorobaculum
k_Bacteria.p_Proteobacteria.c_Gammaproteobacteria.o_Oceanospirillales.f_Oceanospirillaceae.g_Marinospirillum
k_Bacteria.p_Fusobacteria.c_Fusobacteriia.o_Fusobacteriales.f_Leptotrichiaceae.g_Streptobacillus
k_Bacteria.p_Proteobacteria.c_Alphaproteobacteria.o_Rhodobacterales.f_Rhodobacteraceae.g_Oceanibulbus
k_Bacteria.p_Proteobacteria.c_Betaproteobacteria.o_Burkholderiales.f_Comamonadaceae.g_Pelomonas
k_Archaea.p_Euryarchaeota.c_Halobacteria.o_Halobacteriales.f_Halobacteriaceae.g_Halorhabdus
k_Bacteria.p_Bacteroidetes.c_Cytophagia.o_Cytophagales.f_Thermonemataceae.g_Thermonema
k_Bacteria.p_Proteobacteria.c_Betaproteobacteria.o_Nitrosomonadales.f_Nitrosomonadaceae.g_Nitrosospira
k_Bacteria.p_Bacteroidetes.c_Bacteroidia.o_Bacteroidales.f_Bacteroidaceae.g_BacteroideS
k_Bacteria.p_Caldiserica.c_Caldisericia.o_Caldisericales.f_Caldisericaceae.g_Caldisericum
k_Bacteria.p_Cyanobacteria.o_Chroococcales.g_Synechococcus
k_Bacteria.p_Proteobacteria.c_Betaproteobacteria.o_Burkholderiales.g_Aquabacterium
k_Bacteria.p_Proteobacteria.c_Gammaproteobacteria.o_Oceanospirillales.f_Hahellaceae.g_Endozoicomonas
k_Bacteria.p_Proteobacteria.c_Alphaproteobacteria.o_Rhodobacterales.f_Rhodobacteraceae.g_Roseobacter
k_Bacteria.p_Actinobacteria.c_Actinobacteria.o_Micrococcales.f_Promicromonosporaceae.g_Promicromonospora
k_Bacteria.p_Bacteroidetes.c_Flavobacteriia.o_Flavobacteriales.f_Flavobacteriaceae.g_Joostella
k_Bacteria.p_Proteobacteria.c_Gammaproteobacteria.o_Chromatiales.f_Ectothiorhodospiraceae.g_Thioalkalivibrio
k_Bacteria.p_Proteobacteria.c_Betaproteobacteria.o_Burkholderiales.f_Oxalobacteraceae.g_Noviherbaspirillum
k_Bacteria.p_Planctomycetes.c_Planctomycetia.o_Planctomycetales.f_Planctomycetaceae.g_Planctomyces
k_Bacteria.p_Fusobacteria.c_Fusobacteriia.o_Fusobacteriales.f_Leptotrichiaceae.g_Leptotrichia
k_Bacteria.p_Proteobacteria.c_Gammaproteobacteria.o_Nevskiales.f_Sinobacteraceae.g_Solimonas
k_Bacteria.p_Nitrospirae.c_Nitrospira.o_Nitrospirales.f_Nitrospiraceae.g_Candidatus_Magnetobacterium
k_Bacteria.p_Proteobacteria.c_Gammaproteobacteria.o_Enterobacteriales.f_Enterobacteriaceae.g_Photorhabdus
k_Archaea.p_Euryarchaeota.c_Thermoplasmata.o_Thermoplasmatales.f_Ferroplasmaceae.g_Acidiplasma
k_Bacteria.p_Cyanobacteria.o_Chroococcales.g_Halothece
k_Bacteria.p_Proteobacteria.c_Alphaproteobacteria.o_Rhodospirillales.f_Acetobacteraceae.g_Granulibacter
k_Bacteria.p_Proteobacteria.c_Gammaproteobacteria.o_Enterobacteriales.f_Enterobacteriaceae.g_Trabulsiella
k_Bacteria.p_Proteobacteria.c_Betaproteobacteria.o_Burkholderiales.g_Mitsuaria
k_Bacteria.p_Proteobacteria.c_Alphaproteobacteria.o_Caulobacterales.f_Caulobacteraceae.g_Woodsholea
k_Bacteria.p_Proteobacteria.c_Alphaproteobacteria.o_Rickettsiales.f_Anaplasmataceae.g_Anaplasma
k_Bacteria.p_Deinococcus.Thermus.c_Deinococci.o_Deinococcales.f_Deinococcaceae.g_Deinococcus
k_Archaea.p_Euryarchaeota.c_Halobacteria.o_Halobacteriales.f_Halobacteriaceae.g_Candidatus_Halobonum
k_Bacteria.p_Proteobacteria.c_Gammaproteobacteria.o_Enterobacteriales.f_Enterobacteriaceae.g_Arsenophonus
k_Bacteria.p_Proteobacteria.c_Gammaproteobacteria.o_Thiotrichales.f_Francisellaceae.g_Francisella
k_Bacteria.p_Proteobacteria.c_Betaproteobacteria.o_Neisseriales.f_Neisseriaceae.g_Kingella
k_Bacteria.p_Proteobacteria.c_Alphaproteobacteria.o_Rhodospirillales.f_Rhodospirillaceae.g_Rhodospirillum
k_Bacteria.p_Proteobacteria.c_Gammaproteobacteria.o_Xanthomonadales.f_Xanthomonadaceae.g_Xanthomonas
k_Archaea.p_Crenarchaeota.c_Thermoprotei.o_Desulfurococcales.f_Desulfurococcaceae.g_Desulfurococcus
k_Bacteria.p_Actinobacteria.c_Actinobacteria.o_Micrococcales.f_Microbacteriaceae.g_Herbiconiux
k_Bacteria.p_Proteobacteria.c_Gammaproteobacteria.o_Pseudomonadales.f_Pseudomonadaceae.g_Oblitimonas
k_Bacteria.p_Firmicutes.c_Clostridia.o_Clostridiales.f_Lachnospiraceae.g_Butyrivibrio
k_Bacteria.p_Proteobacteria.c_Gammaproteobacteria.o_Alteromonadales.f_Alteromonadaceae.g_Agarivorans
k_Bacteria.p_Firmicutes.c_Negativicutes.o_Veillonellales.f_Veillonellaceae.g_Megasphaera
k_Bacteria.p_Bacteroidetes.c_Bacteroidia.o_Bacteroidales.f_Lentimicrobiaceae.g_Lentimicrobium
k_Bacteria.p_Firmicutes.c_Clostridia.o_Clostridiales.f_Heliobacteriaceae.g_Heliobacterium
k_Bacteria.p_Proteobacteria.c_Gammaproteobacteria.o_Orbales.f_Orbaceae.g_Frischella
k_Bacteria.p_Proteobacteria.c_Alphaproteobacteria.o_Rhizobiales.f_Bradyrhizobiaceae.g_Afipia
k_Archaea.p_Crenarchaeota.c_Thermoprotei.o_Desulfurococcales.f_Desulfurococcaceae.g_Thermogladius
k_Bacteria.p_Lentisphaerae.c_Lentisphaeria.o_Lentisphaerales.f_Lentisphaeraceae.g_Lentisphaera
k_Bacteria.p_Proteobacteria.c_Alphaproteobacteria.o_Rhizobiales.f_Phyllobacteriaceae.g_Nitratireductor
k_Bacteria.p_Proteobacteria.c_Zetaproteobacteria.o_Mariprofundales.f_Mariprofundaceae.g_Mariprofundus
k_Bacteria.p_Proteobacteria.c_Gammaproteobacteria.o_Enterobacteriales.f_Enterobacteriaceae.g_Yokenella
k_Bacteria.p_Bacteroidetes.c_Flavobacteriia.o_Flavobacteriales.f_Flavobacteriaceae.g_Gaetbulibacter
k_Bacteria.p_Firmicutes.c_Clostridia.o_Clostridiales.f_Symbiobacteriaceae.g_Symbiobacterium
k_Bacteria.p_Proteobacteria.c_Gammaproteobacteria.o_Pasteurellales.f_Pasteurellaceae.g_Histophilus
k_Bacteria.p_Proteobacteria.c_Deltaproteobacteria.o_Myxococcales.f_Cystobacteraceae.g_Archangium
k_Bacteria.p_Acidobacteria.c_Acidobacteriia.o_Acidobacteriales.f_Acidobacteriaceae.g_Edaphobacter
k_Archaea.p_Euryarchaeota.c_Halobacteria.o_Halobacteriales.f_Halobacteriaceae.g_Halosimplex
k_Bacteria.p_Actinobacteria.c_Actinobacteria.o_Streptosporangiales.f_Thermomonosporaceae.g_Spirillospora
k_Bacteria.p_Proteobacteria.c_Betaproteobacteria.o_Burkholderiales.g_Paucibacter
k_Bacteria.p_Cyanobacteria.o_Chroococcales.g_Synechocystis
k_Bacteria.p_Proteobacteria.c_Betaproteobacteria.o_Burkholderiales.f_Alcaligenaceae.g_Basilea
k_Bacteria.p_Firmicutes.c_Bacilli.o_Bacillales.f_Alicyclobacillaceae.g_Effusibacillus
k_Bacteria.p_Firmicutes.c_Clostridia.o_Clostridiales.f_Lachnospiraceae.g_Lachnoanaerobaculum
k_Bacteria.p_Proteobacteria.c_Gammaproteobacteria.o_Pasteurellales.f_Pasteurellaceae.g_Avibacterium
k_Bacteria.p_Firmicutes.c_Bacilli.o_Lactobacillales.f_Carnobacteriaceae.g_Jeotgalibaca
k_Bacteria.p_Proteobacteria.c_Alphaproteobacteria.o_Rhizobiales.f_Rhizobiaceae.g_Neorhizobium
k_Bacteria.p_Proteobacteria.c_Betaproteobacteria.o_Neisseriales.f_Chromobacteriaceae.g_Chitinibacter
k_Bacteria.p_Verrucomicrobia.c_Verrucomicrobiae.o_Verrucomicrobiales.f_Verrucomicrobiaceae.g_Verrucomicrobium
k_Bacteria.p_Proteobacteria.c_Betaproteobacteria.o_Burkholderiales.f_Burkholderiaceae.g_Ralstonia
k_Bacteria.p_Proteobacteria.c_Acidithiobacillia.o_Acidithiobacillales.f_Thermithiobacillaceae.g_Thermithiobacillus
k_Bacteria.p_Bacteroidetes.c_Chitinophagia.o_Chitinophagales.f_Chitinophagaceae.g_Flavisolibacter
k_Bacteria.p_Actinobacteria.c_Actinobacteria.o_Corynebacteriales.f_Segniliparaceae.g_Segniliparus
k_Bacteria.p_Firmicutes.c_Clostridia.o_Thermoanaerobacterales.f_Thermoanaerobacterales_Family_III._Incertae_Sedis.g_Syntrophaceticus
k_Bacteria.p_Synergistetes.c_Synergistia.o_Synergistales.f_Synergistaceae.g_Anaerobaculum
k_Bacteria.p_Bacteroidetes.c_Cytophagia.o_Cytophagales.f_Cytophagaceae.g_Fibrella
k_Bacteria.p_Bacteroidetes.c_Flavobacteriia.o_Flavobacteriales.f_Flavobacteriaceae.g_Zunongwangia
k_Bacteria.p_Firmicutes.c_Bacilli.o_Bacillales.f_Planococcaceae.g_Kurthia
k_Bacteria.p_Bacteroidetes.c_Flavobacteriia.o_Flavobacteriales.f_Flavobacteriaceae.g_Gramella
k_Bacteria.p_Chloroflexi.c_Dehalococcoidia.g_Dehalogenimonas
k_Bacteria.p_Candidatus_Cloacimonetes.g_Candidatus_Cloacimonas
k_Bacteria.p_Firmicutes.c_Negativicutes.o_Selenomonadales.f_Selenomonadaceae.g_Anaerovibrio
k_Archaea.p_Euryarchaeota.c_Methanomicrobia.o_Methanomicrobiales.f_Methanoregulaceae.g_Methanolinea
k_Archaea.p_Euryarchaeota.c_Methanomicrobia.o_Methanomicrobiales.f_Methanomicrobiaceae.g_Methanolacinia
k_Bacteria.p_Proteobacteria.c_Alphaproteobacteria.o_Rhodospirillales.f_Rhodospirillaceae.g_Inquilinus
k_Bacteria.p_Synergistetes.c_Synergistia.o_Synergistales.f_Synergistaceae.g_Jonquetella
k_Bacteria.p_Proteobacteria.c_Gammaproteobacteria.o_Alteromonadales.f_Idiomarinaceae.g_Idiomarina
k_Archaea.p_Euryarchaeota.c_Methanococci.o_Methanococcales.f_Methanocaldococcaceae.g_Methanotorris
k_Bacteria.p_Cyanobacteria.o_Pleurocapsales.g_Myxosarcina
k_Bacteria.p_Proteobacteria.c_Betaproteobacteria.o_Neisseriales.f_Chromobacteriaceae.g_Pseudogulbenkiania
k_Bacteria.p_Chloroflexi.c_Anaerolineae.o_Anaerolineales.f_Anaerolineaceae.g_Anaerolinea
k_Bacteria.p_Proteobacteria.c_Gammaproteobacteria.o_Enterobacteriales.f_Enterobacteriaceae.g_Pantoea
k_Bacteria.p_Proteobacteria.c_Alphaproteobacteria.g_Thermopetrobacter
k_Bacteria.p_Actinobacteria.c_Acidimicrobiia.o_Acidimicrobiales.f_Acidimicrobiaceae.g_Ferrimicrobium
k_Bacteria.p_Firmicutes.c_Negativicutes.o_Selenomonadales.f_Selenomonadaceae.g_Setenomonas
k_Bacteria.p_Proteobacteria.c_Gammaproteobacteria.o_Pasteurellales.f_Pasteurellaceae.g_Gallibacterium
k_Bacteria.p_Proteobacteria.c_Gammaproteobacteria.o_Thiotrichales.f_Piscirickettsiaceae.g_Thiomicrospira
k_Bacteria.p_Proteobacteria.c_Gammaproteobacteria.o_Alteromonadales.f_Alteromonadaceae.g_Gayadomonas
k_Bacteria.p_Proteobacteria.c_Alphaproteobacteria.o_Rhodospirillales.f_Rhodospirillaceae.g_Oceanibaculum
k_Bacteria.p_Proteobacteria.c_Gammaproteobacteria.o_Chromatiales.f_Chromatiaceae.g_Thiohalocapsa
k_Bacteria.p_Proteobacteria.c_Alphaproteobacteria.o_Rhizobiales.f_Methylocystaceae.g_Terasakiella
k_Bacteria.p_Proteobacteria.c_Alphaproteobacteria.o_Rhodobacterales.f_Rhodobacteraceae.g_Rubellimicrobium
k_Archaea.p_Euryarchaeota.c_Methanomicrobia.o_Methanosarcinales.f_Methanosarcinaceae.g_Methanohalophilus
k_Bacteria.p_Firmicutes.c_Bacilli.o_Lactobacillales.f_Aerococcaceae.g_Eremococcus
k_Bacteria.p_Actinobacteria.c_Actinobacteria.o_Micrococcales.g_Tropheryma
k_Bacteria.p_Firmicutes.c_Clostridia.o_Clostridiales.f_Lachnospiraceae.g_Blautia
k_Bacteria.p_Proteobacteria.c_Gammaproteobacteria.o_Chromatiales.f_Ectothiorhodospiraceae.g_Acidihalobacter
k_Bacteria.p_Bacteroidetes.c_Flavobacteriia.o_Flavobacteriales.f_Flavobacteriaceae.g_Ornithobacterium
k_Bacteria.p_Proteobacteria.c_Alphaproteobacteria.o_Rhodobacterales.f_Rhodobacteraceae.g_Halocynthiibacter
k_Archaea.p_Thaumarchaeota.c_Nitrososphaeria.o_Nitrososphaerales.f_Nitrososphaeraceae.g_Candidatus_Nitrosocosmicus
k_Bacteria.p_Firmicutes.c_Erysipelotrichia.o_Erysipelotrichales.f_Erysipelotrichaceae.g_Coprobacillus
k_Bacteria.p_Proteobacteria.c_Gammaproteobacteria.o_Enterobacteriales.f_Enterobacteriaceae.g_Buttiauxella
k_Bacteria.p_Proteobacteria.c_Gammaproteobacteria.o_Enterobacteriales.f_Enterobacteriaceae.g_Candidatus_Hamiltonella
k_Bacteria.p_Proteobacteria.c_Betaproteobacteria.o_Burkholderiales.f_Comamonadaceae.g_Pseudacidovorax
k_Bacteria.p_Firmicutes.c_Clostridia.o_Thermoanaerobacterales.f_Thermoanaerobacteraceae.g_Moorella
k_Bacteria.p_Proteobacteria.c_Gammaproteobacteria.o_Methylococcales.f_Methylococcaceae.g_Methylomarinum
k_Archaea.p_Euryarchaeota.c_Halobacteria.o_Natrialbales.f_Natrialbaceae.g_Natronorubrum
k_Archaea.p_Euryarchaeota.c_Halobacteria.o_Haloferacales.f_Haloferacaceae.g_Haloferax
k_Bacteria.p_Firmicutes.c_Bacilli.o_Bacillales.f_Bacillales_Family_X._Incertae_Sedis.g_Thermicanus
k_Bacteria.p_Thermotogae.c_Thermotogae.o_Thermotogales.f_Fervidobacteriaceae.g_Thermosipho
k_Bacteria.p_Proteobacteria.c_Alphaproteobacteria.o_Rhodobacterales.f_Hyphomonadaceae.g_Maricaulis
k_Bacteria.p_Proteobacteria.c_Gammaproteobacteria.o_Enterobacteriales.f_Enterobacteriaceae.g_Candidatus_Riesia
k_Bacteria.p_Proteobacteria.c_Gammaproteobacteria.o_Xanthomonadales.f_Rhodanobacteraceae.g_Rhodanobacter
k_Bacteria.p_Proteobacteria.c_Gammaproteobacteria.o_Alteromonadales.f_Alteromonadaceae.g_Glaciecola
k_Bacteria.p_Proteobacteria.c_Gammaproteobacteria.o_Chromatiales.f_Chromatiaceae.g_Arsukibacterium
k_Bacteria.p_Proteobacteria.c_Alphaproteobacteria.o_Rhodobacterales.f_Rhodobacteraceae.g_Roseibium
k_Bacteria.p_Proteobacteria.c_Gammaproteobacteria.g_Gallaecimonas
k_Bacteria.p_Firmicutes.c_Clostridia.o_Clostridiales.f_Peptococcaceae.g_Desulfotomaculum
k_Bacteria.p_Proteobacteria.c_Gammaproteobacteria.o_Enterobacteriales.f_Enterobacteriaceae.g_Pluralibacter
k_Bacteria.p_Proteobacteria.c_Alphaproteobacteria.o_Rhodobacterales.f_Rhodobacteraceae.g_Pseudophaeobacter
k_Archaea.p_Euryarchaeota.c_Methanobacteria.o_Methanobacteriales.f_Methanobacteriaceae.g_Methanobacterium
k_Bacteria.p_Firmicutes.c_Bacilli.o_Bacillales.f_Planococcaceae.g_Viridibacillus
k_Bacteria.p_Actinobacteria.c_Actinobacteria.o_Streptomycetales.f_Streptomycetaceae.g_Streptacidiphilus
k_Bacteria.p_Chloroflexi.c_Chloroflexia.o_Chloroflexales.f_Chloroflexaceae.g_Chloroflexus
k_Bacteria.p_Aquificae.c_Aquificae.o_Aquificales.f_Hydrogenothermaceae.g_Persephoneha
k_Bacteria.p_Proteobacteria.c_Gammaproteobacteria.o_Cellvibrionales.f_Cellvibrionaceae.g_Teredinibacter
k_Bacteria.p_Firmicutes.c_Erysipelotrichia.o_Erysipelotrichales.f_Erysipelotrichaceae.g_Candidatus_Stoquefichus
k_Bacteria.p_Firmicutes.c_Clostridia.o_Clostridiales.f_Lachnospiraceae.g_Robinsoniella
k_Bacteria.p_Proteobacteria.c_Deltaproteobacteria.g_Deferrisoma
k_Bacteria.p_Chloroflexi.c_Ktedonobacteria.o_Ktedonobacterales.f_Ktedonobacteraceae.g_Ktedonobacter
k_Bacteria.p_Proteobacteria.c_Deltaproteobacteria.o_Myxococcales.f_Sandaracinaceae.g_Sandaracinus
k_Bacteria.p_Proteobacteria.c_Alphaproteobacteria.o_Rhizobiales.f_Aurantimonadaceae.g_Aureimonas
k_Bacteria.p_Firmicutes.c_Clostridia.o_Clostridiales.f_Lachnospiraceae.g_Catonella
k_Bacteria.p_Firmicutes.c_Bacilli.o_Lactobacillales.f_Lactobacillaceae.g_Lactobacillus
k_Bacteria.p_Chrysiogenetes.c_Chrysiogenetes.o_Chrysiogenales.f_Chrysiogenaceae.g_Desulfurispirillum
k_Bacteria.p_Actinobacteria.c_Actinobacteria.o_Streptosporangiales.f_Nocardiopsaceae.g_Streptomonospora
k_Bacteria.p_Firmicutes.c_Clostridia.o_Clostridiales.f_Ruminococcaceae.g_Pseudobacteroides
k_Archaea.p_Euryarchaeota.c_Methanococci.o_Methanococcales.f_Methanococcaceae.g_Methanothermococcus
k_Bacteria.p_Proteobacteria.c_Betaproteobacteria.g_Kinetoplastibacterium
k_Bacteria.p_Proteobacteria.c_Gammaproteobacteria.o_Chromatiales.f_Ectothiorhodospiraceae.g_Thiorhodospira
k_Bacteria.p_Bacteroidetes.c_Cytophagia.o_Cytophagales.f_Hymenobacteraceae.g_Pontibacter
k_Bacteria.p_Proteobacteria.c_Gammaproteobacteria.o_Chromatiales.f_Chromatiaceae.g_Thiocapsa
k_Bacteria.p_Actinobacteria.c_Actinobacteria.o_Micrococcales.f_Microbacteriaceae.g_Agromyces
k_Bacteria.p_Actinobacteria.c_Actinobacteria.o_Pseudonocardiales.f_Pseudonocardiaceae.g_Prauserella
k_Bacteria.p_Bacteroidetes.c_Chitinophagia.o_Chitinophagales.f_Chitinophagaceae.g_Sediminibacterium
k_Bacteria.p_Actinobacteria.c_Actinobacteria.o_Micrococcales.f_Brevibacteriaceae.g_Brevibacterium
k_Bacteria.p_Proteobacteria.c_Alphaproteobacteria.o_Rhizobiales.f_Methylocystaceae.g_Methylopila
k_Bacteria.p_Proteobacteria.c_Alphaproteobacteria.o_Kiloniellales.f_Kiloniellaceae.g_Kiloniella
k_Bacteria.p_Actinobacteria.c_Acidimicrobiia.o_Acidimicrobiales.f_Acidimicrobiaceae.g_Acidithrix
k_Bacteria.p_Proteobacteria.c_Betaproteobacteria.o_Neisseriales.f_Neisseriaceae.g_Alysiella
k_Archaea.p_Euryarchaeota.c_Thermoplasmata.o_Methanomassiliicoccales.f_Methanomassiliicoccaceae.g_Methanomassiliicoccus
k_Bacteria.p_Spirochaetes.c_Spirochaetia.o_Spirochaetales.f_Spirochaetaceae.g_Salinispira
k_Bacteria.p_Firmicutes.c_Clostridia.o_Thermoanaerobacterales.f_Thermodesulfobiaceae.g_Coprothermobacter
k_Bacteria.p_Actinobacteria.c_Coriobacteriia.o_Eggerthellales.f_Eggerthellaceae.g_Cryptobacterium
k_Bacteria.p_Bacteroidetes.c_Cytophagia.o_Cytophagales.f_Cytophagaceae.g_Emticicia
k_Bacteria.p_Proteobacteria.c_Alphaproteobacteria.o_Rhodobacterales.f_Rhodobacteraceae.g_Pelagibaca
k_Bacteria.p_Proteobacteria.c_Betaproteobacteria.g_Candidatus_Accumulibacter
k_Bacteria.p_Proteobacteria.c_Gammaproteobacteria.o_Cardiobacteriales.f_Cardiobacteriaceae.g_Cardiobacterium
k_Bacteria.p_Proteobacteria.c_Deltaproteobacteria.o_Desulfobacterales.f_Desulfobulbaceae.g_Desulfocapsa
k_Bacteria.p_Proteobacteria.c_Alphaproteobacteria.o_Rhizobiales.f_Bradyrhizobiaceae.g_Bosea
k_Bacteria.p_Bacteroidetes.c_Flavobacteriia.o_Flavobacteriales.f_Flavobacteriaceae.g_Psychroflexus
k_Archaea.p_Euryarchaeota.c_Thermococci.o_Thermococcales.f_Thermococcaceae.g_Palaeococcus
k_Archaea.p_Euryarchaeota.c_Methanococci.o_Methanococcales.f_Methanocaldococcaceae.g_Methanocaldococcus
k_Bacteria.p_Cyanobacteria.o_Oscillatoriales.g_Kamptonema
k_Bacteria.p_Actinobacteria.c_Actinobacteria.o_Actinomycetales.f_Actinomycetaceae.g_Flaviflexus
k_Bacteria.p_Firmicutes.c_Bacilli.o_Bacillales.f_Planococcaceae.g_Jeotgalibacillus
k_Bacteria.p_Firmicutes.c_Bacilli.o_Bacillales.f_Paenibacillaceae.g_Brevibacillus
k_Bacteria.p_Proteobacteria.c_Alphaproteobacteria.o_Rhodospirillales.f_Acetobacteraceae.g_Acidiphilium
k_Bacteria.p_Proteobacteria.c_Gammaproteobacteria.o_Enterobacteriales.f_Enterobacteriaceae.g_Chania
k_Bacteria.p_Proteobacteria.c_Gammaproteobacteria.o_Oceanospirillales.f_Halomonadaceae.g_Zymobacter
k_Bacteria.p_Firmicutes.c_Clostridia.o_Clostridiales.f_Clostridiaceae.g_Clostridium
k_Bacteria.p_Proteobacteria.c_Alphaproteobacteria.g_Micavibrio
k_Bacteria.p_Proteobacteria.c_Betaproteobacteria.o_Burkholderiales.f_Sutterellaceae.g_Sutterella
k_Bacteria.p_Actinobacteria.c_Actinobacteria.o_Pseudonocardiales.f_Pseudonocardiaceae.g_Actinomycetospora
k_Bacteria.p_Proteobacteria.c_Gammaproteobacteria.o_Vibrionales.f_Vibrionaceae.g_Photobacterium
k_Bacteria.p_Bacteroidetes.c_Flavobacteriia.o_Flavobacteriales.f_Flavobacteriaceae.g_Aequorivita
k_Bacteria.p_Cyanobacteria.o_Oscillatoriales.g_Trichodesmium
k_Bacteria.p_Proteobacteria.c_Gammaproteobacteria.o_Chromatiales.f_Chromatiaceae.g_Thiorhodococcus
k_Bacteria.p_Proteobacteria.c_Gammaproteobacteria.o_Methylococcales.f_Methylococcaceae.g_Methylobacter
k_Bacteria.p_Chlorobi.c_Chlorobia.o_Chlorobiales.f_Chlorobiaceae.g_Prosthecochloris
k_Bacteria.p_Actinobacteria.c_Actinobacteria.o_Micromonosporales.f_Micromonosporaceae.g_Verrucosispora
k_Bacteria.p_Bacteroidetes.c_Cytophagia.o_Cytophagales.f_Cyclobacteriaceae.g_Algoriphagus
k_Bacteria.p_Bacteroidetes.c_Cytophagia.o_Cytophagales.f_Amoebophilaceae.g_Candidatus_Cardinium
k_Bacteria.p_Proteobacteria.c_Gammaproteobacteria.o_Nevskiales.f_Sinobacteraceae.g_Hydrocarboniphaga
k_Bacteria.p_Cyanobacteria.o_Nostocales.f_Nostocaceae.g_Raphidiopsis
k_Bacteria.p_Actinobacteria.c_Coriobacteriia.o_Eggerthellales.f_Eggerthellaceae.g_Slackia
k_Bacteria.p_Proteobacteria.c_Betaproteobacteria.o_Hydrogenophilales.f_Hydrogenophilaceae.g_Tepidiphilus
k_Bacteria.p_Proteobacteria.c_Alphaproteobacteria.o_Rhizobiales.f_Rhizobiaceae.g_Agrobacterium
k_Bacteria.p_Proteobacteria.c_Alphaproteobacteria.o_Rhizobiales.f_Brucellaceae.g_Ochrobactrum
k_Bacteria.p_Actinobacteria.c_Actinobacteria.o_Micrococcales.f_Micrococcaceae.g_Paeniglutamicibacter
k_Bacteria.p_Proteobacteria.c_Deltaproteobacteria.o_Myxococcales.f_Myxococcaceae.g_Myxococcus
k_Archaea.p_Euryarchaeota.c_Halobacteria.o_Natrialbales.f_Natrialbaceae.g_Natronococcus
k_Archaea.p_Euryarchaeota.c_Halobacteria.o_Haloferacales.f_Haloferacaceae.g_Halobellus
k_Bacteria.p_Proteobacteria.c_Alphaproteobacteria.o_Rhodobacterales.f_Rhodobacteraceae.g_Leisingera
k_Bacteria.p_Proteobacteria.c_Gammaproteobacteria.o_Xanthomonadales.f_Xanthomonadaceae.g_Lysobacter
k_Bacteria.p_Firmicutes.c_Clostridia.o_Clostridiales.f_Lachnospiraceae.g_Stomatobaculum
k_Bacteria.p_Bacteroidetes.c_Flavobacteriia.o_Flavobacteriales.f_Flavobacteriaceae.g_Flaviramulus
k_Bacteria.p_Proteobacteria.c_Alphaproteobacteria.o_Sphingomonadales.f_Sphingomonadaceae.g_Novosphingobium
k_Bacteria.p_Bacteroidetes.c_Bacteroidia.o_Bacteroidales.f_Rikenellaceae.g_Mucinivorans
k_Bacteria.p_Actinobacteria.c_Actinobacteria.o_Micrococcales.f_Dermacoccaceae.g_Luteipulveratus
k_Bacteria.p_Proteobacteria.c_Alphaproteobacteria.o_Rickettsiales.f_Rickettsiaceae.g_Rickettsia
k_Bacteria.p_Firmicutes.c_Bacilli.o_Bacillales.f_Bacillaceae.g_Oceanobacillus
k_Bacteria.p_Firmicutes.c_Clostridia.o_Clostridiales.f_Ruminococcaceae.g_Ethanoligenens
k_Bacteria.g_Thermobaculum
k_Bacteria.p_Firmicutes.c_Clostridia.o_Clostridiales.f_Syntrophomonadaceae.g_Syntrophomonas
k_Bacteria.p_Proteobacteria.c_Gammaproteobacteria.g_Pseudohongiella
k_Archaea.p_Euryarchaeota.c_Archaeoglobi.o_Archaeoglobales.f_Archaeoglobaceae.g_Ferroglobus
k_Bacteria.p_Actinobacteria.c_Actinobacteria.o_Pseudonocardiales.f_Pseudonocardiaceae.g_Kibdelosporangium
k_Bacteria.p_Fusobacteria.c_Fusobacteriia.o_Fusobacteriales.f_Leptotrichiaceae.g_Sebaldella
k_Bacteria.p_Proteobacteria.c_Betaproteobacteria.o_Neisseriales.f_Neisseriaceae.g_Amantichitinum
k_Bacteria.p_Bacteroidetes.c_Cytophagia.o_Cytophagales.f_Cytophagaceae.g_Cytophaga
k_Bacteria.p_Proteobacteria.c_Gammaproteobacteria.o_Xanthomonadales.f_Rhodanobacteraceae.g_Rudaea
k_Bacteria.p_Proteobacteria.c_Gammaproteobacteria.o_Cellvibrionales.f_Cellvibrionaceae.g_Marinimicrobium
k_Bacteria.p_Bacteroidetes.c_Cytophagia.o_Cytophagales.f_Cyclobacteriaceae.g_Cyclobacterium
k_Bacteria.p_Actinobacteria.c_Actinobacteria.o_Catenulisporales.f_Catenulisporaceae.g_Catenulispora
k_Bacteria.p_Firmicutes.c_Clostridia.o_Clostridiales.f_Peptostreptococcaceae.g_Filifactor
k_Bacteria.p_Proteobacteria.c_Gammaproteobacteria.o_Enterobacteriales.f_Enterobacteriaceae.g_Candidatus_Moranella
k_Bacteria.p_Bacteroidetes.c_Sphingobacteriia.o_Sphingobacteriales.f_Sphingobacteriaceae.g_Olivibacter
k_Bacteria.p_Chloroflexi.c_Thermomicrobia.o_Sphaerobacterales.f_Sphaerobacteraceae.g_Sphaerobacter
k_Bacteria.p_Proteobacteria.c_Gammaproteobacteria.o_Oceanospirillales.f_Oceanospirillaceae.g_Marinobacterium
k_Bacteria.p_Proteobacteria.c_Deltaproteobacteria.o_Desulfuromonadales.f_Geobacteraceae.g_Geopsychrobacter
k_Bacteria.p_Proteobacteria.c_Betaproteobacteria.o_Burkholderiales.g_Ideonella
k_Bacteria.p_Proteobacteria.c_Gammaproteobacteria.o_Nevskiales.f_Algiphilaceae.g_Algiphilus
k_Bacteria.p_Proteobacteria.c_Gammaproteobacteria.o_Enterobacteriales.f_Enterobacteriaceae.g_Candidatus_Blochmannia
k_Archaea.p_Euryarchaeota.c_Halobacteria.o_Natrialbales.f_Natrialbaceae.g_Halovivax
k_Bacteria.p_Firmicutes.c_Clostridia.o_Clostridiales.f_Ruminococcaceae.g_Ruminococcus
k_Bacteria.p_Proteobacteria.c_Betaproteobacteria.o_Gallionellales.f_Gallionellaceae.g_Sideroxydans
k_Bacteria.p_Chlorobi.c_Chlorobia.o_Chlorobiales.f_Chlorobiaceae.g_Chloroherpeton
k_Archaea.p_Euryarchaeota.g_Aciduliprofundum
k_Archaea.p_Crenarchaeota.c_Thermoprotei.o_Desulfurococcales.f_Desulfurococcaceae.g_Ignicoccus
k_Bacteria.p_Firmicutes.c_Bacilli.o_Bacillales.f_Bacillaceae.g_Sediminibacillus
k_Bacteria.p_Actinobacteria.c_Coriobacteriia.o_Eggerthellales.f_Eggerthellaceae.g_Enterorhabdus
k_Bacteria.p_Actinobacteria.c_Actinobacteria.o_Micrococcales.f_Promicromonosporaceae.g_Isoptericola
k_Bacteria.p_Bacteroidetes.c_Flavobacteriia.o_Flavobacteriales.f_Flavobacteriaceae.g_Cellulophaga
k_Bacteria.p_Proteobacteria.c_Deltaproteobacteria.o_Myxococcales.f_Vulgatibacteraceae.g_Vulgatibacter
k_Bacteria.p_Proteobacteria.c_Alphaproteobacteria.o_Rhizobiales.f_Rhodobiaceae.g_Lutibaculum
k_Bacteria.p_Bacteroidetes.c_Flavobacteriia.o_Flavobacteriales.f_Flavobacteriaceae.g_Tamlana
k_Archaea.p_Euryarchaeota.c_Halobacteria.o_Natrialbales.f_Natrialbaceae.g_Natrialba
k_Bacteria.p_Firmicutes.c_Bacilli.o_Lactobacillales.f_Leuconostocaceae.g_Leuconostoc
k_Bacteria.p_Firmicutes.c_Tissierellia.o_Tissierellales.f_Peptoniphilaceae.g_Helcococcus
k_Bacteria.p_Proteobacteria.c_Alphaproteobacteria.o_Sphingomonadales.f_Sphingomonadaceae.g_Citromicrobium
k_Bacteria.p_Actinobacteria.c_Coriobacteriia.o_Coriobacteriales.f_Coriobacteriaceae.g_Enorma
k_Bacteria.p_Firmicutes.c_Bacilli.o_Bacillales.f_Alicyclobacillaceae.g_Alicyclobacillus
k_Bacteria.p_Proteobacteria.c_Alphaproteobacteria.o_Rhodobacterales.f_Rhodobacteraceae.g_Litoreibacter
k_Bacteria.p_Proteobacteria.c_Alphaproteobacteria.o_Rhodospirillales.f_Rhodospirillaceae.g_Magnetospira
k_Bacteria.p_Firmicutes.c_Clostridia.o_Clostridiales.f_Ruminococcaceae.g_Mageeibacillus
k_Bacteria.p_Proteobacteria.c_Gammaproteobacteria.o_Oceanospirillales.f_Saccharospirillaceae.g_Gynuella
k_Bacteria.p_Actinobacteria.c_Actinobacteria.o_Frankiales.f_Cryptosporangiaceae.g_Cryptosporangium
k_Bacteria.p_Spirochaetes.c_Spirochaetia.o_Spirochaetales.f_Spirochaetaceae.g_Sphaerochaeta
k_Bacteria.p_Proteobacteria.c_Alphaproteobacteria.o_Rickettsiales.f_Candidatus_Paracaedibacteraceae.g_Candidatus_Paracaedibacter
k_Bacteria.p_Bacteroidetes.c_Bacteroidia.o_Bacteroidales.f_Porphyromonadaceae.g_Dysgonomonas
k_Bacteria.p_Spirochaetes.c_Spirochaetia.o_Spirochaetales.f_Borreliaceae.g_Borreliella
k_Bacteria.p_Proteobacteria.c_Betaproteobacteria.o_Ferrovales.f_Ferrovaceae.g_Ferrovum
k_Bacteria.p_Firmicutes.c_Clostridia.o_Clostridiales.f_Peptostreptococcaceae.g_Paraclostridium
k_Bacteria.p_Proteobacteria.c_Deltaproteobacteria.o_Myxococcales.f_Cystobacteraceae.g_Stigmatella
k_Bacteria.p_Firmicutes.c_Bacilli.o_Lactobacillales.f_Carnobacteriaceae.g_Alloiococcus
k_Bacteria.p_Bacteroidetes.c_Cytophagia.o_Cytophagales.f_Flammeovirgaceae.g_Nafulseha
k_Archaea.p_Euryarchaeota.c_Methanomicrobia.o_Methanosarcinales.f_Methanosarcinaceae.g_Methanohalobium
k_Bacteria.p_Proteobacteria.c_Gammaproteobacteria.o_Aeromonadales.f_Succinivibrionaceae.g_Succinimonas
k_Bacteria.p_Proteobacteria.c_Gammaproteobacteria.o_Thiotrichales.f_Piscirickettsiaceae.g_Thioalkalimicrobium
k_Bacteria.p_Proteobacteria.c_Gammaproteobacteria.o_Chromatiales.f_Halothiobacillaceae.g_Halothiobacillus
k_Bacteria.p_Actinobacteria.c_Actinobacteria.o_Micrococcales.f_Microbacteriaceae.g_Microterricola
k_Bacteria.p_Fusobacteria.c_Fusobacteriia.o_Fusobacteriales.f_Fusobacteriaceae.g_Ilyobacter
k_Bacteria.p_Proteobacteria.c_Gammaproteobacteria.o_Oceanospirillales.f_Saccharospirillaceae.g_Saccharospirillum
k_Bacteria.p_Planctomycetes.c_Planctomycetia.o_Planctomycetales.f_Planctomycetaceae.g_Planctopirus
k_Bacteria.p_Proteobacteria.c_Betaproteobacteria.o_Burkholderiales.g_Xylophilus
k_Bacteria.p_Bacteroidetes.c_Bacteroidia.o_Bacteroidales.g_Phocaeicola
k_Bacteria.p_Proteobacteria.c_Gammaproteobacteria.o_Oceanospirillales.f_Oceanospirillaceae.g_Nitrincola
k_Bacteria.p_Firmicutes.c_Bacilli.o_Bacillales.f_Paenibacillaceae.g_Aneurinibacillus
k_Bacteria.p_Proteobacteria.c_Gammaproteobacteria.f_Competibacteraceae.g_Candidatus_Contendobacter
k_Bacteria.p_Firmicutes.c_Clostridia.o_Clostridiales.g_Epulopiscium
k_Bacteria.p_Firmicutes.c_Bacilli.o_Bacillales.f_Paenibacillaceae.g_Gorillibacterium
k_Bacteria.p_Actinobacteria.c_Actinobacteria.o_Micrococcales.f_Microbacteriaceae.g_Candidatus_Aquiluna
k_Bacteria.p_Proteobacteria.c_Gammaproteobacteria.o_Cellvibrionales.f_Cellvibrionaceae.g_Saccharophagus
k_Bacteria.p_Acidobacteria.c_Blastocatellia.g_Pyrinomonas
k_Bacteria.p_Proteobacteria.c_Gammaproteobacteria.o_Thiotrichales.f_Piscirickettsiaceae.g_Piscirickettsia
k_Archaea.p_Euryarchaeota.c_Thermococci.o_Thermococcales.f_Thermococcaceae.g_Thermococcus
k_Bacteria.p_Proteobacteria.c_Betaproteobacteria.o_Gallionellales.f_Gallionellaceae.g_Ferriphaselus
k_Bacteria.p_Proteobacteria.c_Alphaproteobacteria.o_Rhodobacterales.f_Rhodobacteraceae.g_Rhodobacter
k_Bacteria.p_Proteobacteria.c_Epsilonproteobacteria.o_Campylobacterales.f_Campylobacteraceae.g_Campylobacter
k_Bacteria.p_Bacteroidetes.c_Flavobacteriia.o_Flavobacteriales.f_Flavobacteriaceae.g_Robiginitalea
k_Bacteria.p_Proteobacteria.c_Deltaproteobacteria.g_Candidatus_Babela
k_Bacteria.p_Cyanobacteria.o_Nostocales.f_Nostocaceae.g_Cylindrospermum
k_Bacteria.p_Proteobacteria.c_Gammaproteobacteria.o_Enterobacteriales.f_Enterobacteriaceae.g_Shimwellia
k_Bacteria.p_Bacteroidetes.c_Flavobacteriia.o_Flavobacteriales.f_Flavobacteriaceae.g_Elizabethkingia
k_Bacteria.p_Armatimonadetes.c_Fimbriimonadia.o_Fimbriimonadales.f_Fimbriimonadaceae.g_Fimbriimonas
k_Bacteria.p_Actinobacteria.c_Actinobacteria.o_Micrococcales.f_Cellulomonadaceae.g_Actinotalea
k_Bacteria.p_Cyanobacteria.o_Stigonematales.g_Mastigocoleus
k_Bacteria.p_Ignavibacteriae.c_Ignavibacteria.o_Ignavibacteriales.f_Ignavibacteriaceae.g_Ignavibacterium
k_Bacteria.p_Bacteroidetes.c_Flavobacteriia.o_Flavobacteriales.f_Flavobacteriaceae.g_Nonlabens
k_Bacteria.p_Bacteroidetes.c_Cytophagia.o_Cytophagales.f_Cyclobacteriaceae.g_Cecembia
k_Bacteria.p_Proteobacteria.c_Alphaproteobacteria.o_Rhizobiales.f_Methylobacteriaceae.g_Methylobacterium
k_Bacteria.p_Actinobacteria.c_Actinobacteria.o_Kineosporiales.f_Kineosporiaceae.g_Angustibacter
k_Bacteria.p_Proteobacteria.c_Gammaproteobacteria.o_Methylococcales.f_Methylococcaceae.g_Methylomonas
k_Bacteria.p_Proteobacteria.c_Alphaproteobacteria.o_Rhodospirillales.f_Acetobacteraceae.g_Gluconobacter
k_Bacteria.p_Firmicutes.c_Bacilli.o_Bacillales.f_Staphylococcaceae.g_Macrococcus
k_Bacteria.p_Proteobacteria.c_Gammaproteobacteria.o_Alteromonadales.f_Alteromonadaceae.g_Paraglaciecola
k_Bacteria.p_Cyanobacteria.o_Prochlorales.f_Prochlorotrichaceae.g_Prochlorothrix
k_Bacteria.p_Proteobacteria.c_Gammaproteobacteria.o_Methylococcales.f_Methylococcaceae.g_ethylococcus
k_Bacteria.p_Deferribacteres.c_Deferribacteres.o_Deferribacterales.f_Deferribacteraceae.g_Calditerrivibrio
k_Bacteria.p_Proteobacteria.c_Betaproteobacteria.o_Burkholderiales.f_Alcaligenaceae.g_Oligella
k_Bacteria.p_Proteobacteria.c_Gammaproteobacteria.o_Cellvibrionales.f_Halieaceae.g_Luminiphilus
k_Bacteria.p_Thermodesulfobacteria.c_Thermodesulfobacteria.o_Thermodesulfobacteriales.f_Thermodesulfobacteriaceae.g_Caldimicrobium
k_Bacteria.p_Proteobacteria.c_Betaproteobacteria.o_Neisseriales.f_Neisseriaceae.g_Stenoxybacter
k_Bacteria.p_Cyanobacteria.o_Prochlorales.f_Prochlorococcaceae.g_Prochlorococcus
k_Bacteria.p_Proteobacteria.c_Alphaproteobacteria.o_Rhodospirillales.f_Rhodospirillaceae.g_Niveispirillum
k_Bacteria.p_Firmicutes.c_Clostridia.o_Clostridiales.f_Clostridiales_Family_XVII._Incertae_Sedis.g_Sulfobacillus
k_Bacteria.p_Bacteroidetes.c_Cytophagia.o_Cytophagales.f_Hymenobacteraceae.g_Rufibacter
k_Bacteria.p_Proteobacteria.c_Gammaproteobacteria.o_Alteromonadales.f_Alteromonadaceae.g_Aestuariibacter
k_Bacteria.p_Firmicutes.c_Bacilli.o_Bacillales.f_Listeriaceae.g_Brochothrix
k_Bacteria.p_Proteobacteria.c_Gammaproteobacteria.o_Oceanospirillales.f_Oceanospirillaceae.g_Oceanospirillum
k_Bacteria.p_Actinobacteria.c_Actinobacteria.o_Propionibacteriales.f_Nocardioidaceae.g_Actinopolymorpha
k_Bacteria.p_Proteobacteria.c_Alphaproteobacteria.o_Rhodospirillales.g_Elioraea
k_Bacteria.p_Proteobacteria.c_Betaproteobacteria.o_Neisseriales.f_Chromobacteriaceae.g_Microvirgula
k_Bacteria.p_Actinobacteria.c_Actinobacteria.o_Actinomycetales.f_Actinomycetaceae.g_Actinomyces
k_Bacteria.p_Proteobacteria.c_Gammaproteobacteria.o_Pseudomonadales.f_Moraxellaceae.g_Acinetobacter
k_Bacteria.p_Proteobacteria.c_Alphaproteobacteria.o_Rhodobacterales.f_Rhodobacteraceae.g_Sediminimonas
k_Bacteria.p_Proteobacteria.c_Gammaproteobacteria.o_Legionellales.f_Legionellaceae.g_Fluoribacter
k_Bacteria.p_Cyanobacteria.o_Oscillatoriales.g_Spirulina
k_Bacteria.p_Proteobacteria.c_Gammaproteobacteria.g_Candidatus_Ruthia
k_Bacteria.p_Actinobacteria.c_Actinobacteria.o_Micrococcales.f_Micrococcaceae.g_Nesterenkonia
k_Bacteria.p_Proteobacteria.c_Gammaproteobacteria.o_Thiotrichales.f_Piscirickettsiaceae.g_Cycloclasticus
k_Bacteria.p_Proteobacteria.c_Alphaproteobacteria.o_Rhodospirillales.f_Rhodospirillaceae.g_Haematospirillum
k_Bacteria.p_Bacteroidetes.c_Cytophagia.o_Cytophagales.f_Cytophagaceae.g_Flexibacter
k_Bacteria.p_Proteobacteria.c_Alphaproteobacteria.o_Rhodobacterales.f_Rhodobacteraceae.g_Defluviimonas
k_Bacteria.p_Proteobacteria.c_Alphaproteobacteria.o_Rhizobiales.f_Rhodobiaceae.g_Candidatus_Phaeomarinobacter
k_Bacteria.p_Proteobacteria.c_Alphaproteobacteria.o_Rhodospirillales.f_Rhodospirillaceae.g_Novispirillum
k_Bacteria.p_Acidobacteria.c_Acidobacteriia.o_Acidobacteriales.f_Acidobacteriaceae.g_Acidobacterium
k_Bacteria.p_Proteobacteria.c_Gammaproteobacteria.f_Competibacteraceae.g_Candidatus_Competibacter
k_Bacteria.p_Firmicutes.c_Bacilli.o_Lactobacillales.f_Lactobacillaceae.g_Pediococcus
k_Bacteria.p_Firmicutes.c_Clostridia.o_Clostridiales.f_Lachnospiraceae.g_Lachnobacterium
k_Archaea.p_Euryarchaeota.c_Thermoplasmata.o_Methanomassiliicoccales.f_Methanomassiliicoccaceae.g_Candidatus_Methanoplasma
k_Bacteria.p_Firmicutes.c_Clostridia.o_Clostridiales.f_Eubacteriaceae.g_Anaerofustis
k_Bacteria.p_Proteobacteria.c_Gammaproteobacteria.o_Thiotrichales.g_Fangia
k_Bacteria.p_Proteobacteria.c_Gammaproteobacteria.o_Nevskiales.f_Sinobacteraceae.g_Nevskia
k_Bacteria.p_Proteobacteria.c_Gammaproteobacteria.o_Enterobacteriales.f_Enterobacteriaceae.g_Xenorhabdus
k_Bacteria.p_Proteobacteria.c_Gammaproteobacteria.o_Xanthomonadales.f_Xanthomonadaceae.g_Stenotrophomonas
k_Bacteria.p_Firmicutes.c_Bacilli.o_Bacillales.f_Planococcaceae.g_Bhargavaea
k_Bacteria.p_Proteobacteria.c_Alphaproteobacteria.o_Rhizobiales.f_Beijerinckiaceae.g_Methylocella
k_Bacteria.p_Proteobacteria.c_Betaproteobacteria.g_Candidatus_Profftella
k_Bacteria.p_Proteobacteria.c_Alphaproteobacteria.o_Sphingomonadales.f_Erythrobacteraceae.g_Croceicoccus
k_Bacteria.p_Proteobacteria.c_Gammaproteobacteria.o_Enterobacteriales.f_Enterobacteriaceae.g_Plesiomonas
k_Bacteria.p_Actinobacteria.c_Actinobacteria.o_Corynebacteriales.f_Mycobacteriaceae.g_Amycolicicoccus
k_Bacteria.p_Proteobacteria.c_Betaproteobacteria.o_Neisseriales.f_Neisseriaceae.g_Snodgrassella
k_Archaea.p_Crenarchaeota.c_Thermoprotei.o_Desulfurococcales.f_Pyrodictiaceae.g_Pyrodictium
k_Bacteria.p_Proteobacteria.c_Alphaproteobacteria.o_Rhodobacterales.f_Rhodobacteraceae.g_Sulfitobacter
k_Bacteria.p_Actinobacteria.c_Coriobacteriia.o_Coriobacteriales.f_Coriobacteriaceae.g_Senegalimassilia
k_Bacteria.p_Proteobacteria.c_Gammaproteobacteria.o_Xanthomonadales.f_Rhodanobacteraceae.g_Mizugakiibacter
k_Bacteria.p_Actinobacteria.c_Actinobacteria.o_Micrococcales.f_Dermatophilaceae.g_Austwickia
k_Bacteria.p_Firmicutes.c_Bacilli.o_Bacillales.f_Bacillaceae.g_Fictibacillus
k_Bacteria.p_Proteobacteria.c_Betaproteobacteria.o_Neisseriales.f_Neisseriaceae.g_Vitreoscilla
k_Bacteria.p_Firmicutes.c_Bacilli.o_Bacillales.f_Sporolactobacillaceae.g_Sporolactobacillus
k_Bacteria.p_Proteobacteria.c_Gammaproteobacteria.o_Oceanospirillales.f_Hahellaceae.g_Hahella
k_Bacteria.p_Bacteroidetes.c_Cytophagia.o_Cytophagales.f_Cyclobacteriaceae.g_Lunatimonas
k_Bacteria.p_Proteobacteria.c_Gammaproteobacteria.o_Enterobacteriales.f_Enterobacteriaceae.g_Erwinia
k_Bacteria.p_Bacteroidetes.c_Flavobacteriia.o_Flavobacteriales.f_Flavobacteriaceae.g_Muricauda
k_Bacteria.p_Verrucomicrobia.c_Spartobacteria.g_Terrimicrobium
k_Bacteria.p_Firmicutes.c_Erysipelotrichia.o_Erysipelotrichales.f_Erysipelotrichaceae.g_Turicibacter
k_Bacteria.p_Acidobacteria.c_Acidobacteriia.o_Acidobacteriales.f_Acidobacteriaceae.g_Candidatus_Koribacter
k_Bacteria.p_Proteobacteria.c_Betaproteobacteria.o_Burkholderiales.f_Comamonadaceae.g_Pseudorhodoferax
k_Bacteria.p_Actinobacteria.c_Actinobacteria.o_Micrococcales.f_Intrasporangiaceae.g_Janibacter
k_Bacteria.p_Actinobacteria.c_Actinobacteria.o_Micrococcales.f_Dermabacteraceae.g_Helcobacillus
k_Bacteria.p_Proteobacteria.c_Betaproteobacteria.o_Gallionellales.f_Gallionellaceae.g_Gallionella
k_Archaea.p_Crenarchaeota.c_Thermoprotei.o_Desulfurococcales.f_Pyrodictiaceae.g_Pyrolobus
k_Bacteria.p_Firmicutes.c_Negativicutes.o_Selenomonadales.f_Selenomonadaceae.g_Centipeda
k_Bacteria.p_Actinobacteria.c_Actinobacteria.o_Corynebacteriales.f_Mycobacteriaceae.g_Mycobacterium
k_Archaea.p_Euryarchaeota.c_Methanococci.o_Methanococcales.f_Methanococcaceae.g_Methanococcus
k_Bacteria.p_Proteobacteria.c_Alphaproteobacteria.g_Candidatus_Puniceispirillum
k_Bacteria.p_Firmicutes.c_Bacilli.o_Bacillales.f_Bacillaceae.g_Bacillus
k_Bacteria.p_Actinobacteria.c_Actinobacteria.o_Streptosporangiales.f_Streptosporangiaceae.g_Planomonospora
k_Bacteria.p_Firmicutes.c_Clostridia.o_Clostridiales.f_Clostridiales_Family_XIII._Incertae_Sedis.g_Anaerovorax
k_Bacteria.p_Bacteroidetes.c_Bacteroidia.o_Bacteroidales.f_Marinilabiliaceae.g_Alkaliflexus
k_Bacteria.p_Verrucomicrobia.c_Verrucomicrobiae.o_Verrucomicrobiales.f_Rubritaleaceae.g_Rubritalea
k_Bacteria.p_Bacteroidetes.c_Flavobacteriia.o_Flavobacteriales.f_Flavobacteriaceae.g_Mangrovimonas
k_Bacteria.p_Chloroflexi.c_Ktedonobacteria.o_Thermogemmatisporales.f_Thermogemmatisporaceae.g_Thermogemmatispora
k_Bacteria.p_Firmicutes.c_Clostridia.o_Clostridiales.f_Ruminococcaceae.g_Anaerotruncus
k_Bacteria.p_Bacteroidetes.c_Flavobacteriia.o_Flavobacteriales.f_Flavobacteriaceae.g_Empedobacter
k_Bacteria.p_Proteobacteria.c_Gammaproteobacteria.o_Enterobacteriales.f_Enterobacteriaceae.g_Kosakonia
k_Bacteria.p_Firmicutes.c_Negativicutes.o_Selenomonadales.f_Selenomonadaceae.g_Propionispira
k_Bacteria.p_Proteobacteria.c_Gammaproteobacteria.o_Oceanospirillales.f_Halomonadaceae.g_Candidatus_Evansia
k_Archaea.p_Euryarchaeota.c_Halobacteria.o_Halobacteriales.f_Halobacteriaceae.g_Hatapricum
k_Bacteria.p_Actinobacteria.c_Actinobacteria.o_Micrococcales.f_Microbacteriaceae.g_Rhodoluna
k_Bacteria.p_Actinobacteria.c_Actinobacteria.o_Micrococcales.f_Microbacteriaceae.g_Gryllotalpicola
k_Bacteria.p_Proteobacteria.c_Alphaproteobacteria.o_Rhodospirillales.f_Rhodospirillaceae.g_Thalassobaculum
k_Bacteria.p_Proteobacteria.c_Deltaproteobacteria.o_Bdellovibrionales.f_Halobacteriovoraceae.g_Halobacteriovorax
k_Bacteria.p_Firmicutes.c_Negativicutes.o_Veillonellales.f_Veillonellaceae.g_Dialister
k_Bacteria.p_Verrucomicrobia.o_Methylacidiphilales.f_Methylacidiphilaceae.g_Methylacidiphilum
k_Bacteria.p_Actinobacteria.c_Actinobacteria.o_Micrococcales.f_Dermatophilaceae.g_Kineosphaera
k_Bacteria.p_Actinobacteria.c_Actinobacteria.o_Streptosporangiales.f_Nocardiopsaceae.g_Allosalinactinospora
k_Bacteria.p_Firmicutes.c_Erysipelotrichia.o_Erysipelotrichales.f_Erysipelotrichaceae.g_Faecalicoccus
k_Bacteria.p_Verrucomicrobia.c_Opitutae.o_Opitutales.f_Opitutaceae.g_Opitutus
k_Bacteria.p_Planctomycetes.c_Planctomycetia.o_Planctomycetales.f_Planctomycetaceae.g_Gimesia
k_Bacteria.p_Firmicutes.c_Bacilli.o_Bacillales.f_Planococcaceae.g_Sporosarcina
k_Bacteria.p_Proteobacteria.c_Gammaproteobacteria.o_Chromatiales.f_Wenzhouxiangellaceae.g_Wenzhouxiangella
k_Bacteria.p_Actinobacteria.c_Actinobacteria.o_Corynebacteriales.g_Lawsonella
k_Bacteria.p_Proteobacteria.c_Gammaproteobacteria.o_Oceanospirillales.f_Halomonadaceae.g_Candidatus_Portiera
k_Bacteria.p_Firmicutes.c_Erysipelotrichia.o_Erysipelotrichales.f_Erysipelotrichaceae.g_Holdemanella
k_Archaea.p_Euryarchaeota.c_Methanomicrobia.o_Methanomicrobiales.f_Methanomicrobiaceae.g_Methanomicrobium
k_Bacteria.p_Bacteroidetes.c_Bacteroidia.o_Bacteroidales.f_Porphyromonadaceae.g_Sanguibacteroides
k_Bacteria.p_Proteobacteria.c_Alphaproteobacteria.o_Rhizobiales.f_Hyphomicrobiaceae.g_Hyphomicrobium
k_Bacteria.p_Proteobacteria.c_Alphaproteobacteria.o_Rhizobiales.f_Bradyrhizobiaceae.g_Tardiphaga
k_Bacteria.p_Firmicutes.c_Negativicutes.o_Acidaminococcales.f_Acidaminococcaceae.g_Acidaminococcus
k_Bacteria.p_Proteobacteria.c_Alphaproteobacteria.o_Sphingomonadales.f_Sphingomonadaceae.g_Sphingopyxis
k_Bacteria.p_Actinobacteria.c_Actinobacteria.o_Micrococcales.f_Intrasporangiaceae.g_Terracoccus
k_Bacteria.p_Proteobacteria.c_Gammaproteobacteria.o_Xanthomonadales.f_Xanthomonadaceae.g_Arenimonas
k_Bacteria.p_Proteobacteria.c_Alphaproteobacteria.o_Rhodobacterales.f_Rhodobacteraceae.g_Ponticoccus
k_Bacteria.p_Actinobacteria.c_Actinobacteria.o_Propionibacteriales.f_Propionibacteriaceae.g_Microlunatus
k_Bacteria.p_Actinobacteria.c_Actinobacteria.o_Micrococcales.f_Intrasporangiaceae.g_Knoellia
k_Bacteria.p_Actinobacteria.c_Actinobacteria.o_Pseudonocardiales.f_Pseudonocardiaceae.g_Sciscionella
k_Bacteria.p_Proteobacteria.c_Deltaproteobacteria.o_Desulfobacterales.f_Desulfobulbaceae.g_Desulfotalea
k_Bacteria.p_Proteobacteria.c_Betaproteobacteria.o_Burkholderiales.f_Burkholderiaceae.g_Chitinimonas
k_Bacteria.p_Firmicutes.c_Clostridia.o_Clostridiales.f_Lachnospiraceae.g_Tyzzerella
k_Bacteria.p_Cyanobacteria.o_Nostocales.f_Scytonemataceae.g_Scytonema
k_Bacteria.p_Proteobacteria.c_Gammaproteobacteria.o_Pseudomonadales.f_Moraxellaceae.g_Moraxella
k_Bacteria.p_Proteobacteria.c_Alphaproteobacteria.o_Magnetococcales.f_Magnetococcaceae.g_Magnetococcus
k_Archaea.p_Euryarchaeota.c_Halobacteria.o_Haloferacales.f_Haloferacaceae.g_Halogranum
k_Bacteria.p_Firmicutes.c_Clostridia.o_Thermoanaerobacterales.f_Thermoanaerobacteraceae.g_Tepidanaerobacter
k_Bacteria.p_Bacteroidetes.c_Cytophagia.o_Cytophagales.f_Flammeovirgaceae.g_Roseivirga
k_Bacteria.p_Proteobacteria.c_Alphaproteobacteria.o_Rhizobiales.f_Hyphomicrobiaceae.g_Rhodoplanes
k_Bacteria.p_Proteobacteria.c_Gammaproteobacteria.g_Thiolapillus
k_Bacteria.p_Actinobacteria.c_Actinobacteria.o_Micrococcales.f_Cellulomonadaceae.g_Paraoerskovia
k_Bacteria.p_Cyanobacteria.o_Pleurocapsales.g_Xenococcus
k_Bacteria.p_Chloroflexi.c_Caldilineae.o_Caldilineales.f_Caldilineaceae.g_Caldilinea
k_Bacteria.p_Proteobacteria.c_Gammaproteobacteria.o_Xanthomonadales.f_Rhodanobacteraceae.g_Luteibacter
k_Bacteria.p_Proteobacteria.c_Betaproteobacteria.o_Neisseriales.f_Chromobacteriaceae.g_Andreprevotia
k_Bacteria.p_Bacteroidetes.c_Cytophagia.o_Cytophagales.f_Flammeovirgaceae.g_Flexithrix
k_Bacteria.p_Cyanobacteria.o_Oscillatoriales.g_Coleofasciculus
k_Bacteria.p_Proteobacteria.c_Gammaproteobacteria.o_Thiotrichales.f_Thiotrichaceae.g_Thioploca
k_Bacteria.p_Firmicutes.c_Clostridia.o_Halanaerobiales.f_Halobacteroidaceae.g_Orenia
k_Bacteria.p_Actinobacteria.c_Actinobacteria.o_Pseudonocardiales.f_Pseudonocardiaceae.g_Actinokineospora
k_Archaea.p_Euryarchaeota.c_Thermococci.o_Thermococcales.f_Thermococcaceae.g_Pyrococcus
k_Bacteria.p_Actinobacteria.c_Actinobacteria.o_Streptosporangiales.f_Nocardiopsaceae.g_Thermobifida
k_Bacteria.p_Proteobacteria.c_Epsilonproteobacteria.o_Nautiliales.f_Nautiliaceae.g_Nautiha
k_Archaea.p_Euryarchaeota.c_Methanomicrobia.o_Methanosarcinales.f_Methanosarcinaceae.g_Methanosalsum
k_Bacteria.p_Proteobacteria.c_Betaproteobacteria.o_Burkholderiales.f_Alcaligenaceae.g_Derxia
k_Archaea.p_Thaumarchaeota.g_Candidatus_Nitrosopelagicus
k_Bacteria.p_Bacteroidetes.c_Flavobacteriia.o_Flavobacteriales.f_Crocinitomicaceae.g_Fluviicola
k_Bacteria.p_Firmicutes.c_Clostridia.o_Clostridiales.g_Intestinimonas
k_Bacteria.p_Firmicutes.c_Bacilli.o_Bacillales.f_Staphylococcaceae.g_Salinicoccus
k_Archaea.p_Euryarchaeota.c_Halobacteria.o_Halobacteriales.f_Halobacteriaceae.g_Halomicrobium
k_Bacteria.p_Proteobacteria.c_Deltaproteobacteria.o_Syntrophobacterales.f_Syntrophaceae.g_Syntrophus
k_Bacteria.p_Firmicutes.c_Negativicutes.o_Selenomonadales.f_Sporomusaceae.g_Sporomusa
k_Bacteria.p_Actinobacteria.c_Actinobacteria.o_Micrococcales.f_Dermatophilaceae.g_Mobilicoccus
k_Bacteria.p_Proteobacteria.c_Alphaproteobacteria.o_Rhodospirillales.f_Rhodospirillaceae.g_Rhodovibrio
k_Bacteria.p_Proteobacteria.c_Gammaproteobacteria.o_Enterobacteriales.f_Enterobacteriaceae.g_Obesumbacterium
k_Bacteria.p_Aquificae.c_Aquificae.o_Desulfurobacteriales.f_Desulfurobacteriaceae.g_Thermovibrio
k_Bacteria.p_Bacteroidetes.c_Flavobacteriia.o_Flavobacteriales.f_Flavobacteriaceae.g_Mesoflavibacter
k_Bacteria.p_Bacteroidetes.c_Sphingobacteriia.o_Sphingobacteriales.f_Sphingobacteriaceae.g_Pedobacter
k_Bacteria.p_Proteobacteria.c_Deltaproteobacteria.o_Myxococcales.f_Polyangiaceae.g_Chondromyces
k_Bacteria.p_Firmicutes.c_Clostridia.o_Clostridiales.g_Levyella
k_Bacteria.p_Actinobacteria.c_Actinobacteria.o_Propionibacteriales.f_Propionibacteriaceae.g_Propionibacterium
k_Bacteria.p_Proteobacteria.c_Gammaproteobacteria.o_Chromatiales.f_Ectothiorhodospiraceae.g_Alkalilimnicola
k_Bacteria.p_Firmicutes.c_Erysipelotrichia.o_Erysipelotrichales.f_Erysipelotrichaceae.g_Kandleria
k_Bacteria.p_Bacteroidetes.c_Cytophagia.o_Cytophagales.f_Cytophagaceae.g_Spirosoma
k_Bacteria.p_Proteobacteria.c_Gammaproteobacteria.o_Enterobacteriales.f_Enterobacteriaceae.g_Shigella
k_Bacteria.p_Deferribacteres.c_Deferribacteres.o_Deferribacterales.f_Deferribacteraceae.g_Denitrovibrio
k_Bacteria.p_Proteobacteria.c_Gammaproteobacteria.o_Chromatiales.f_Ectothiorhodospiraceae.g_Spiribacter
k_Bacteria.p_Bacteroidetes.c_Chitinophagia.o_Chitinophagales.f_Saprospiraceae.g_Aureispira
k_Bacteria.p_Proteobacteria.c_Gammaproteobacteria.o_Xanthomonadales.f_Rhodanobacteraceae.g_Dyella
k_Bacteria.p_Proteobacteria.c_Alphaproteobacteria.o_Rhodobacterales.f_Rhodobacteraceae.g_Roseovarius
k_Bacteria.p_Proteobacteria.c_Gammaproteobacteria.o_Aeromonadales.f_Succinivibrionaceae.g_Succinivibrio
k_Bacteria.p_Proteobacteria.c_Gammaproteobacteria.o_Oceanospirillales.f_Oceanospirillaceae.g_Oleispira
k_Bacteria.p_Proteobacteria.c_Alphaproteobacteria.o_Rhodobacterales.f_Rhodobacteraceae.g_Aliiroseovarius
k_Bacteria.p_Bacteroidetes.c_Chitinophagia.o_Chitinophagales.f_Chitinophagaceae.g_Flavihumibacter
k_Bacteria.p_Bacteroidetes.c_Flavobacteriia.o_Flavobacteriales.f_Flavobacteriaceae.g_Polaribacter
k_Bacteria.p_Proteobacteria.c_Deltaproteobacteria.o_Desulfovibrionales.f_Desulfovibrionaceae.g_Bilophila
k_Bacteria.p_Proteobacteria.c_Alphaproteobacteria.o_Rhizobiales.f_Hyphomicrobiaceae.g_Rhodomicrobium
k_Bacteria.p_Bacteroidetes.c_Flavobacteriia.o_Flavobacteriales.f_Flavobacteriaceae.g_Winogradskyella
k_Bacteria.p_Proteobacteria.c_Alphaproteobacteria.o_Rhodobacterales.f_Hyphomonadaceae.g_Euryhalocaulis
k_Bacteria.p_Actinobacteria.c_Actinobacteria.o_Actinomycetales.f_Actinomycetaceae.g_Trueperella
k_Bacteria.p_Actinobacteria.c_Actinobacteria.o_Micrococcales.f_Dermacoccaceae.g_Dermacoccus
k_Bacteria.p_Spirochaetes.c_Spirochaetia.o_Spirochaetales.f_Spirochaetaceae.g_Treponema
k_Bacteria.p_Actinobacteria.c_Actinobacteria.o_Pseudonocardiales.f_Pseudonocardiaceae.g_Pseudonocardia
k_Bacteria.p_Bacteroidetes.c_Flavobacteriia.o_Flavobacteriales.f_Flavobacteriaceae.g_Vitellibacter
k_Bacteria.p_Actinobacteria.c_Actinobacteria.o_Corynebacteriales.f_Nocardiaceae.g_Smaragdicoccus
k_Bacteria.p_Actinobacteria.c_Actinobacteria.o_Corynebacteriales.f_Williamsiaceae.g_Williamsia
k_Bacteria.p_Proteobacteria.c_Alphaproteobacteria.o_Rhodobacterales.f_Rhodobacteraceae.g_Actibacterium
k_Bacteria.p_Spirochaetes.c_Spirochaetia.o_Brachyspirales.f_Brachyspiraceae.g_Brachyspira
k_Bacteria.p_Firmicutes.c_Clostridia.o_Clostridiales.f_Ruminococcaceae.g_Ruminiclostridium
k_Bacteria.p_Proteobacteria.c_Betaproteobacteria.o_Burkholderiales.f_Comamonadaceae.g_Simplicispira
k_Bacteria.p_Proteobacteria.c_Alphaproteobacteria.o_Rhodobacterales.f_Rhodobacteraceae.g_Nesiotobacter
k_Bacteria.p_Bacteroidetes.c_Flavobacteriia.o_Flavobacteriales.f_Flavobacteriaceae.g_Xanthomarina
k_Bacteria.p_Actinobacteria.c_Actinobacteria.o_Micrococcales.f_Sanguibacteraceae.g_Sanguibacter
k_Archaea.p_Euryarchaeota.c_Halobacteria.o_Halobacteriales.f_Halobacteriaceae.g_Halobacterium
k_Bacteria.p_Proteobacteria.c_Deltaproteobacteria.o_Syntrophobacterales.f_Syntrophaceae.g_Desulfomonile
k_Bacteria.p_Proteobacteria.c_Gammaproteobacteria.o_Pseudomonadales.f_Moraxellaceae.g_Enhydrobacter
k_Bacteria.p_Firmicutes.c_Erysipelotrichia.o_Erysipelotrichales.f_Erysipelotrichaceae.g_Eggerthia
k_Bacteria.p_Cyanobacteria.o_Nostocales.f_Nostocaceae.g_Cylindrospermopsis
k_Bacteria.p_Actinobacteria.c_Actinobacteria.o_Propionibacteriales.f_Propionibacteriaceae.g_Tessaracoccus
k_Bacteria.p_Proteobacteria.c_Alphaproteobacteria.o_Rhodobacterales.f_Rhodobacteraceae.g_Sagittula
k_Bacteria.p_Bacteroidetes.c_Flavobacteriia.o_Flavobacteriales.f_Flavobacteriaceae.g_Kordia
k_Bacteria.p_Proteobacteria.c_Gammaproteobacteria.o_Enterobacteriales.f_Enterobacteriaceae.g_Brenneria
k_Bacteria.p_Proteobacteria.c_Alphaproteobacteria.o_Rhizobiales.f_Aurantimonadaceae.g_Aurantimonas
k_Bacteria.p_Verrucomicrobia.c_Verrucomicrobiae.o_Verrucomicrobiales.f_Verrucomicrobiaceae.g_Haloferula
k_Bacteria.p_Aquificae.c_Aquificae.o_Aquificales.g_Thermosulfidibacter
k_Bacteria.p_Cyanobacteria.o_Stigonematales.g_Mastigocladopsis
k_Bacteria.p_Proteobacteria.c_Deltaproteobacteria.o_Desulfobacterales.f_Desulfobacteraceae.g_Desulforegula
k_Bacteria.p_Proteobacteria.c_Gammaproteobacteria.o_Pasteurellales.f_Pasteurellaceae.g_Chelonobacter
k_Bacteria.p_Actinobacteria.c_Actinobacteria.o_Propionibacteriales.f_Nocardioidaceae.g_Kribbella
k_Bacteria.p_Proteobacteria.c_Alphaproteobacteria.o_Rhizobiales.f_Hyphomicrobiaceae.g_Devosia
k_Bacteria.p_Bacteroidetes.c_Flavobacteriia.o_Flavobacteriales.f_Flavobacteriaceae.g_Lutibacter
k_Bacteria.p_Cyanobacteria.o_Nostocales.f_Nostocaceae.g_Richelia
k_Bacteria.p_Thermodesulfobacteria.c_Thermodesulfobacteria.o_Thermodesulfobacteriales.f_Thermodesulfobacteriaceae.g_Thermodesulfatator
k_Bacteria.p_Thermotogae.c_Thermotogae.o_Thermotogales.f_Thermotogaceae.g_Thermotoga
k_Bacteria.p_Actinobacteria.c_Actinobacteria.o_Micromonosporales.f_Micromonosporaceae.g_Pilimelia
k_Bacteria.p_Proteobacteria.c_Betaproteobacteria.o_Burkholderiales.f_Comamonadaceae.g_Diaphorobacter
k_Bacteria.p_Proteobacteria.c_Alphaproteobacteria.o_Rhodobacterales.f_Rhodobacteraceae.g_Confluentimicrobium
k_Archaea.p_Thaumarchaeota.o_Nitrosopumilales.f_Nitrosopumilaceae.g_Candidatus_Nitrosoarchaeum
k_Bacteria.p_Proteobacteria.c_Betaproteobacteria.o_Burkholderiales.f_Comamonadaceae.g_Brachymonas
k_Bacteria.p_Proteobacteria.c_Alphaproteobacteria.o_Sphingomonadales.f_Erythrobacteraceae.g_Altererythrobacter
k_Bacteria.p_Bacteroidetes.c_Bacteroidia.o_Bacteroidales.f_Marinilabiliaceae.g_Saccharicrinis
k_Bacteria.p_Actinobacteria.c_Actinobacteria.o_Pseudonocardiales.f_Pseudonocardiaceae.g_Actinoalloteichus
k_Bacteria.p_Actinobacteria.c_Thermoleophilia.o_Solirubrobacterales.f_Patulibacteraceae.g_Patulibacter
k_Bacteria.p_Synergistetes.c_Synergistia.o_Synergistales.f_Synergistaceae.g_AminomonaS
k_Bacteria.p_Actinobacteria.c_Actinobacteria.o_Actinomycetales.f_Actinomycetaceae.g_Actinobaculum
k_Bacteria.p_Actinobacteria.c_Actinobacteria.o_Micrococcales.f_Microbacteriaceae.g_Curtobacterium
k_Bacteria.p_Firmicutes.c_Clostridia.o_Clostridiales.f_Clostridiales_Family_XIII._Incertae_Sedis.g_Mogibacterium
k_Bacteria.p_Proteobacteria.c_Alphaproteobacteria.o_Rhodospirillales.f_Acetobacteraceae.g_Saccharibacter
k_Bacteria.p_Proteobacteria.c_Betaproteobacteria.o_Burkholderiales.f_Comamonadaceae.g_Lampropedia
k_Bacteria.p_Thermotogae.c_Thermotogae.o_Mesoaciditogales.f_Mesoaciditogaceae.g_Mesoaciditoga
k_Bacteria.p_Proteobacteria.c_Alphaproteobacteria.o_Rhodobacterales.f_Rhodobacteraceae.g_Phaeobacter
k_Bacteria.p_Acidobacteria.c_Acidobacteriia.o_Acidobacteriales.f_Acidobacteriaceae.g_Silvibacterium
k_Bacteria.p_Thermotogae.c_Thermotogae.o_Thermotogales.f_Fervidobacteriaceae.g_Fervidobacterium
k_Bacteria.p_Proteobacteria.c_Alphaproteobacteria.o_Rhizobiales.f_Xanthobacteraceae.g_Starkeya
k_Bacteria.p_Bacteroidetes.c_Chitinophagia.o_Chitinophagales.f_Chitinophagaceae.g_Terrimonas
k_Bacteria.p_Firmicutes.c_Bacilli.o_Bacillales.f_Alicyclobacillaceae.g_Kyrpidia
k_Bacteria.p_Firmicutes.c_Bacilli.o_Bacillales.f_Sporolactobacillaceae.g_Tuberibacillus
k_Bacteria.p_Proteobacteria.c_Alphaproteobacteria.o_Kordiimonadales.f_Kordiimonadaceae.g_Kordiimonas
k_Bacteria.p_Firmicutes.c_Negativicutes.o_Selenomonadales.f_Sporomusaceae.g_Acetonema
k_Bacteria.p_Proteobacteria.c_Deltaproteobacteria.o_Bdellovibrionales.f_Bdellovibrionaceae.g_Bdellovibrio
k_Bacteria.p_Proteobacteria.c_Betaproteobacteria.o_Burkholderiales.f_Comamonadaceae.g_Hydrogenophaga
k_Bacteria.p_Bacteroidetes.c_Cytophagia.o_Cytophagales.f_Cytophagaceae.g_Sporocytophaga
k_Bacteria.p_Proteobacteria.c_Deltaproteobacteria.o_Syntrophobacterales.f_Syntrophaceae.g_Desulfobacca
k_Bacteria.p_Bacteroidetes.c_Bacteroidia.o_Bacteroidales.f_Prolixibacteraceae.g_Prolixibacter
k_Bacteria.p_Bacteroidetes.c_Bacteroidia.o_Bacteroidales.f_Marinilabiliaceae.g_Marinilabilia
k_Bacteria.p_Proteobacteria.c_Betaproteobacteria.o_Burkholderiales.f_Comamonadaceae.g_Ramlibacter
k_Bacteria.p_Proteobacteria.c_Epsilonproteobacteria.o_Campylobacterales.f_Helicobacteraceae.g_Wolinella
k_Bacteria.p_Proteobacteria.c_Alphaproteobacteria.o_Rhodobacterales.f_Rhodobacteraceae.g_Labrenzia
k_Bacteria.p_Proteobacteria.c_Alphaproteobacteria.o_Rhodospirillales.f_Acetobacteraceae.g_Komagataeibacter
k_Bacteria.p_Proteobacteria.c_Gammaproteobacteria.o_Methylococcales.f_Methylococcaceae.g_Methylosarcina
k_Bacteria.p_Bacteroidetes.c_Flavobacteriia.o_Flavobacteriales.f_Flavobacteriaceae.g_Croceitalea
k_Bacteria.p_Proteobacteria.c_Alphaproteobacteria.o_Rhizobiales.f_Rhizobiaceae.g_Candidatus_Liberibacter
k_Bacteria.p_Actinobacteria.c_Actinobacteria.o_Micrococcales.f_Microbacteriaceae.g_Pseudoclavibacter
k_Bacteria.p_Proteobacteria.c_Betaproteobacteria.o_Burkholderiales.g_Aquincola
k_Bacteria.p_Actinobacteria.c_Actinobacteria.o_Micrococcales.g_Timonella
k_Archaea.p_Crenarchaeota.c_Thermoprotei.o_Thermoproteales.f_Thermoproteaceae.g_Thermoproteus
k_Bacteria.p_Proteobacteria.c_Alphaproteobacteria.o_Rickettsiales.f_Candidatus_Midichloriaceae.g_Candidatus_Midichloria
k_Bacteria.p_Firmicutes.c_Bacilli.o_Bacillales.f_Bacillaceae.g_Geobacillus
k_Bacteria.p_Firmicutes.c_Clostridia.o_Clostridiales.f_Peptostreptococcaceae.g_Peptostreptococcus
k_Bacteria.p_Proteobacteria.c_Betaproteobacteria.o_Burkholderiales.f_Comamonadaceae.g_Curvibacter
k_Bacteria.p_Proteobacteria.c_Epsilonproteobacteria.g_Nitratiruptor
k_Bacteria.p_Firmicutes.c_Bacilli.o_Lactobacillales.f_Aerococcaceae.g_Aerococcus
k_Bacteria.p_Planctomycetes.c_Planctomycetia.o_Planctomycetales.f_Planctomycetaceae.g_Rhodopirellula
k_Bacteria.p_Proteobacteria.c_Deltaproteobacteria.o_Desulfobacterales.f_Desulfobacteraceae.g_Desulfatirhabdium
k_Bacteria.p_Proteobacteria.c_Betaproteobacteria.o_Burkholderiales.f_Alcaligenaceae.g_Castellaniella
k_Bacteria.p_Chrysiogenetes.c_Chrysiogenetes.o_Chrysiogenales.f_Chrysiogenaceae.g_Chrysiogenes
k_Bacteria.p_Proteobacteria.c_Alphaproteobacteria.o_Rhodospirillales.f_Rhodospirillaceae.g_Azospirillum
k_Bacteria.p_Proteobacteria.c_Alphaproteobacteria.o_Caulobacterales.f_Caulobacteraceae.g_Phenylobacterium
k_Bacteria.p_Firmicutes.c_Bacilli.o_Bacillales.f_Bacillaceae.g_Aeribacillus
k_Bacteria.p_Actinobacteria.c_Acidimicrobiia.o_Acidimicrobiales.f_Microthrixaceae.g_Candidatus_Microthrix
k_Bacteria.p_Thermotogae.c_Thermotogae.o_Thermotogales.f_Thermotogaceae.g_Pseudothermotoga
k_Bacteria.p_Nitrospirae.c_Nitrospira.o_Nitrospirales.f_Nitrospiraceae.g_Nitrospira
k_Bacteria.p_Proteobacteria.c_Gammaproteobacteria.g_Sedimenticola
k_Bacteria.p_Firmicutes.c_Clostridia.o_Clostridiales.g_Gottschalkia
k_Bacteria.p_Spirochaetes.c_Spirochaetia.f_Leptospiraceae.g_Turneriella
k_Bacteria.p_Bacteroidetes.c_Bacteroidia.o_Bacteroidales.f_Porphyromonadaceae.g_Porphyromonas
k_Bacteria.p_Proteobacteria.c_Alphaproteobacteria.o_Rhizobiales.f_Rhizobiaceae.g_Rhizobium
k_Bacteria.p_Proteobacteria.c_Alphaproteobacteria.o_Rhizobiales.f_Phyllobacteriaceae.g_Paramesorhizobium
k_Bacteria.p_Firmicutes.c_Clostridia.o_Clostridiales.f_Peptococcaceae.g_Syntrophobotulus
k_Bacteria.p_Firmicutes.c_Bacilli.o_Lactobacillales.f_Enterococcaceae.g_TetragenococcuS
k_Bacteria.p_Actinobacteria.c_Actinobacteria.o_Micrococcales.f_Micrococcaceae.g_Renibacterium
k_Bacteria.p_Firmicutes.c_Negativicutes.o_Selenomonadales.f_Sporomusaceae.g_Anaeromusa
k_Bacteria.p_Firmicutes.c_Clostridia.o_Clostridiales.f_Ruminococcaceae.g_Candidatus_Soleaferrea
k_Bacteria.p_Actinobacteria.c_Actinobacteria.o_Micrococcales.f_Microbacteriaceae.g_Frigoribacterium
k_Archaea.p_Euryarchaeota.c_Halobacteria.o_Natrialbales.f_Natrialbaceae.g_Halopiger
k_Bacteria.p_Firmicutes.c_Clostridia.o_Halanaerobiales.f_Halobacteroidaceae.g_Halobacteroides
k_Bacteria.p_Bacteroidetes.c_Flavobacteriia.o_Flavobacteriales.f_Flavobacteriaceae.g_Sediminicola
k_Bacteria.p_Actinobacteria.c_Actinobacteria.o_Micrococcales.f_Dermatophilaceae.g_Dermatophilus
k_Bacteria.p_Proteobacteria.c_Alphaproteobacteria.o_Rhodospirillales.f_Rhodospirillaceae.g_Dongia
k_Archaea.p_Thaumarchaeota.o_Nitrosopumilales.f_Nitrosopumilaceae.g_Nitrosopumilus
k_Bacteria.p_Firmicutes.c_Bacilli.o_Bacillales.f_Bacillaceae.g_Marinococcus
k_Bacteria.p_Proteobacteria.c_Gammaproteobacteria.o_Pasteurellales.f_Pasteurellaceae.g_Muribacter
k_Bacteria.p_Spirochaetes.c_Spirochaetia.f_Leptospiraceae.g_Leptospira
k_Bacteria.p_Proteobacteria.c_Deltaproteobacteria.o_Desulfobacterales.f_Desulfobacteraceae.g_Desulfobacula
k_Bacteria.p_Firmicutes.c_Bacilli.o_Bacillales.f_Paenibacillaceae.g_Cohnella
k_Archaea.p_Euryarchaeota.c_Methanomicrobia.o_Methanomicrobiales.f_Methanomicrobiaceae.g_Methanoculleus
k_Bacteria.p_Bacteroidetes.o_Bacteroidetes_Order_II._Incertae_sedis.f_Rhodothermaceae.g_Salinibacter
k_Bacteria.p_Proteobacteria.c_Gammaproteobacteria.o_Enterobacteriales.f_Enterobacteriaceae.g_Enterobacter
k_Bacteria.p_Proteobacteria.c_Alphaproteobacteria.o_Rhodobacterales.f_Rhodobacteraceae.g_Oceaniovalibus
k_Bacteria.p_Proteobacteria.c_Gammaproteobacteria.o_Cellvibrionales.f_Spongiibacteraceae.g_Dasania
k_Bacteria.p_Proteobacteria.c_Betaproteobacteria.o_Burkholderiales.f_Comamonadaceae.g_Acidovorax
k_Bacteria.p_Aquificae.c_Aquificae.o_Aquificales.f_Aquificaceae.g_Aquifex
k_Bacteria.p_Bacteroidetes.c_Cytophagia.o_Cytophagales.f_Hymenobacteraceae.g_Adhaeribacter
k_Bacteria.p_Proteobacteria.c_Gammaproteobacteria.o_Alteromonadales.f_Moritellaceae.g_Moritella
k_Bacteria.p_Bacteroidetes.c_Flavobacteriia.o_Flavobacteriales.f_Flavobacteriaceae.g_Myroides
k_Bacteria.p_Proteobacteria.c_Gammaproteobacteria.o_Alteromonadales.f_Alteromonadaceae.g_Alishewanella
k_Bacteria.p_Firmicutes.c_Bacilli.o_Bacillales.g_Exiguobacterium
k_Bacteria.p_Proteobacteria.c_Gammaproteobacteria.o_Methylococcales.f_Methylococcaceae.g_Methylomicrobium
k_Bacteria.p_Proteobacteria.c_Alphaproteobacteria.o_Rhodobacterales.f_Rhodobacteraceae.g_Haematobacter
k_Bacteria.p_Proteobacteria.c_Deltaproteobacteria.o_Myxococcales.f_Anaeromyxobacteraceae.g_Anaeromyxobacter
k_Bacteria.p_Proteobacteria.c_Alphaproteobacteria.o_Rhodobacterales.f_Rhodobacteraceae.g_Nautella
k_Bacteria.p_Proteobacteria.c_Betaproteobacteria.o_Burkholderiales.f_Burkholderiaceae.g_Candidatus_Glomeribacter
k_Bacteria.p_Firmicutes.c_Clostridia.o_Clostridiales.f_Peptococcaceae.g_Thermincola
k_Bacteria.p_Bacteroidetes.c_Chitinophagia.o_Chitinophagales.f_Chitinophagaceae.g_Segetibacter
k_Bacteria.p_Actinobacteria.c_Actinobacteria.o_Micrococcales.f_Microbacteriaceae.g_Glaciibacter
k_Bacteria.p_Firmicutes.c_Bacilli.o_Bacillales.f_Bacillaceae.g_Amphibacillus
k_Bacteria.p_Actinobacteria.c_Actinobacteria.o_Glycomycetales.f_Glycomycetaceae.g_Stackebrandtia
k_Bacteria.p_Spirochaetes.c_Spirochaetia.f_Leptospiraceae.g_Leptonema
k_Bacteria.p_Proteobacteria.c_Alphaproteobacteria.o_Rickettsiales.f_Anaplasmataceae.g_Candidatus_Neoehrlichia
k_Bacteria.p_Proteobacteria.c_Gammaproteobacteria.o_Alteromonadales.f_Colwelliaceae.g_Thalassomonas
k_Bacteria.p_Chlorobi.c_Chlorobia.o_Chlorobiales.f_Chlorobiaceae.g_Chlorobium
k_Bacteria.p_Firmicutes.c_Bacilli.o_Bacillales.f_Planococcaceae.g_Paenisporosarcina
k_Bacteria.p_Planctomycetes.c_Planctomycetia.o_Planctomycetales.f_Planctomycetaceae.g_Gemmata
k_Bacteria.p_Actinobacteria.c_Actinobacteria.o_Frankiales.f_Frankiaceae.g_Frankia
k_Bacteria.p_Bacteroidetes.c_Flavobacteriia.o_Flavobacteriales.f_Flavobacteriaceae.g_Gillista
k_Bacteria.p_Proteobacteria.c_Alphaproteobacteria.o_Sphingomonadales.f_Erythrobacteraceae.g_Porphyrobacter
k_Bacteria.p_Acidobacteria.c_Acidobacteriia.o_Acidobacteriales.f_Acidobacteriaceae.g_Terriglobus
k_Bacteria.p_Firmicutes.c_Tissierellia.o_Tissierellales.f_Peptoniphilaceae.g_Parvimonas
k_Bacteria.p_Elusimicrobia.c_Elusimicrobia.o_Elusimicrobiales.f_Elusimicrobiaceae.g_Elusimicrobium
k_Bacteria.p_Proteobacteria.c_Alphaproteobacteria.o_Rhizobiales.f_Beijerinckiaceae.g_Methylocapsa
k_Bacteria.p_Actinobacteria.c_Actinobacteria.o_Micrococcales.f_Microbacteriaceae.g_Plantibacter
k_Bacteria.p_Proteobacteria.c_Alphaproteobacteria.o_Sphingomonadales.f_Sphingomonadaceae.g_Sphingomonas
k_Bacteria.p_Firmicutes.c_Clostridia.o_Clostridiales.f_Clostridiaceae.g_Thermobrachium
k_Bacteria.p_Planctomycetes.c_Planctomycetia.o_Candidatus_Brocadiales.f_Candidatus_Brocadiaceae.g_Candidatus_Jettenia
k_Bacteria.p_Bacteroidetes.c_Bacteroidia.o_Bacteroidales.f_Odoribacteraceae.g_Odoribacter
k_Bacteria.p_Proteobacteria.c_Gammaproteobacteria.o_Pseudomonadales.f_Ventosimonadaceae.g_Ventosimonas
k_Bacteria.p_Actinobacteria.c_Coriobacteriia.o_Coriobacteriales.f_Atopobiaceae.g_Olsenella
k_Bacteria.p_Firmicutes.c_Clostridia.o_Clostridiales.f_Lachnospiraceae.g_Shuttleworthia
k_Bacteria.p_Proteobacteria.c_Gammaproteobacteria.o_Alteromonadales.f_Alteromonadaceae.g_Marinobacter
k_Bacteria.p_Proteobacteria.c_Gammaproteobacteria.o_Aeromonadales.f_Aeromonadaceae.g_Tolumonas
k_Bacteria.p_Cyanobacteria.o_Oscillatoriales.g_Geitlerinema
k_Bacteria.p_Proteobacteria.c_Alphaproteobacteria.o_Rickettsiales.g_Candidatus_Hepatobacter
k_Bacteria.p_Actinobacteria.c_Actinobacteria.o_Micrococcales.f_Microbacteriaceae.g_Gulosibacter
k_Bacteria.p_Proteobacteria.c_Alphaproteobacteria.o_Rhodospirillales.f_Rhodospirillaceae.g_Magnetospirillum
k_Bacteria.p_Firmicutes.c_Bacilli.o_Lactobacillales.f_Leuconostocaceae.g_Weissella
k_Bacteria.p_Firmicutes.c_Bacilli.o_Bacillales.f_Staphylococcaceae.g_Jeotgalicoccus
k_Bacteria.p_Proteobacteria.c_Deltaproteobacteria.o_Desulfovibrionales.f_Desulfovibrionaceae.g_Desulfovibrio
k_Bacteria.p_Proteobacteria.c_Gammaproteobacteria.o_Xanthomonadales.f_Xanthomonadaceae.g_Xylella
k_Bacteria.p_Proteobacteria.c_Deltaproteobacteria.o_Desulfovibrionales.f_Desulfohalobiaceae.g_Desulfovermiculus
k_Bacteria.p_Proteobacteria.c_Alphaproteobacteria.o_Rhodobacterales.f_Rhodobacteraceae.g_Paenirhodobacter
k_Bacteria.p_Firmicutes.c_Bacilli.o_Bacillales.f_Paenibacillaceae.g_Saccharibacillus
k_Bacteria.p_Proteobacteria.c_Alphaproteobacteria.o_Rhodobacterales.f_Rhodobacteraceae.g_Puniceibacterium
k_Bacteria.p_Bacteroidetes.c_Bacteroidia.o_Bacteroidales.f_Rikenellaceae.g_Alistipes
k_Bacteria.p_Cyanobacteria.o_Chroococcales.g_Rubidibacter
k_Bacteria.p_Actinobacteria.c_Actinobacteria.o_Micromonosporales.f_Micromonosporaceae.g_Asanoa
k_Bacteria.p_Deferribacteres.c_Deferribacteres.o_Deferribacterales.f_Deferribacteraceae.g_Caldithrix
k_Bacteria.p_Actinobacteria.c_Actinobacteria.o_Pseudonocardiales.f_Pseudonocardiaceae.g_Thermocrispum
k_Bacteria.p_Actinobacteria.c_Actinobacteria.o_Actinomycetales.f_Actinomycetaceae.g_Arcanobacterium
k_Bacteria.p_Cyanobacteria.o_Oscillatoriales.g_Nodosilinea
k_Bacteria.p_Actinobacteria.c_Actinobacteria.o_Propionibacteriales.f_Nocardioidaceae.g_Pimelobacter
k_Bacteria.p_Actinobacteria.c_Actinobacteria.o_Micrococcales.f_Dermacoccaceae.g_Kytococcus
k_Bacteria.p_Proteobacteria.c_Betaproteobacteria.o_Methylophilales.f_Methylophilaceae.g_Methylovorus
k_Bacteria.p_Proteobacteria.c_Alphaproteobacteria.o_Rhizobiales.f_Bartonellaceae.g_Bartonella
k_Archaea.p_Euryarchaeota.c_Methanomicrobia.o_Methanosarcinales.f_Candidatus_Methanoperedenaceae.g_Candidatus_Methanoperedens
k_Bacteria.p_Actinobacteria.c_Actinobacteria.o_Micrococcales.f_Micrococcaceae.g_Mierococcus
k_Bacteria.p_Chloroflexi.c_Anaerolineae.o_Anaerolineales.f_Anaerolineaceae.g_Bellilinea
k_Bacteria.p_Proteobacteria.c_Betaproteobacteria.o_Burkholderiales.f_Alcaligenaceae.g_Bordetella
k_Bacteria.p_Proteobacteria.c_Deltaproteobacteria.o_Desulfuromonadales.f_Desulfuromonadaceae.g_Pelobacter
k_Bacteria.p_Proteobacteria.c_Alphaproteobacteria.o_Rhizobiales.f_Hyphomicrobiaceae.g_Cucumibacter
k_Bacteria.p_Proteobacteria.c_Alphaproteobacteria.o_Rhodobacterales.f_Rhodobacteraceae.g_Pseudooceanicola
k_Bacteria.p_Proteobacteria.c_Alphaproteobacteria.o_Rhodobacterales.f_Rhodobacteraceae.g_Sedimentitalea
k_Bacteria.p_Proteobacteria.c_Betaproteobacteria.o_Burkholderiales.f_Comamonadaceae.g_Xenophilus
k_Bacteria.p_Chloroflexi.c_Anaerolineae.o_Anaerolineales.f_Anaerolineaceae.g_Thermanaerothrix
k_Bacteria.p_Proteobacteria.c_Deltaproteobacteria.o_Desulfovibrionales.f_Desulfovibrionaceae.g_Lawsonia
k_Archaea.p_Euryarchaeota.c_Halobacteria.o_Halobacteriales.f_Halobacteriaceae.g_Hatanaeroarchaeum
k_Bacteria.p_Proteobacteria.c_Alphaproteobacteria.o_Rhizobiales.f_Hyphomicrobiaceae.g_Blastochloris
k_Bacteria.p_Proteobacteria.c_Gammaproteobacteria.o_Enterobacteriales.f_Enterobacteriaceae.g_Moellerella
k_Bacteria.p_Proteobacteria.c_Gammaproteobacteria.o_Oceanospirillales.f_Oceanospirillaceae.g_Marinomonas
k_Bacteria.p_Firmicutes.c_Clostridia.o_Clostridiales.f_Christensenellaceae.g_Christensenella
k_Bacteria.p_Bacteroidetes.c_Flavobacteriia.o_Flavobacteriales.f_Flavobacteriaceae.g_Arenibacter
k_Bacteria.p_Thermotogae.c_Thermotogae.o_Kosmotogales.f_Kosmotogaceae.g_Kosmotoga
k_Bacteria.p_Verrucomicrobia.c_Opitutae.o_Puniceicoccales.f_Puniceicoccaceae.g_Coraliomargarita
k_Bacteria.p_Actinobacteria.c_Actinobacteria.o_Kineosporiales.f_Kineosporiaceae.g_Kineosporia
k_Bacteria.p_Proteobacteria.c_Alphaproteobacteria.o_Rhizobiales.f_Beijerinckiaceae.g_Chelatococcus
k_Bacteria.p_Proteobacteria.c_Gammaproteobacteria.o_Cellvibrionales.f_Cellvibrionaceae.g_Cellvibrio
k_Bacteria.p_Actinobacteria.c_Rubrobacteria.o_Rubrobacterales.f_Rubrobacteraceae.g_Rubrobacter
k_Bacteria.p_Proteobacteria.c_Alphaproteobacteria.o_Rhodobacterales.f_Rhodobacteraceae.g_Thalassobium
k_Bacteria.p_Cyanobacteria.o_Nostocales.f_Microchaetaceae.g_Microchaete
k_Bacteria.p_Actinobacteria.c_Actinobacteria.o_Micrococcales.f_Intrasporangiaceae.g_Ornithinimicrobium
k_Bacteria.p_Bacteroidetes.c_Bacteroidia.o_Bacteroidales.f_Porphyromonadaceae.g_Tannerella
k_Bacteria.p_Chloroflexi.c_Thermomicrobia.o_Thermomicrobiales.f_Thermomicrobiaceae.g_Thermomicrobium
k_Bacteria.p_Planctomycetes.c_Planctomycetia.o_Planctomycetales.f_Planctomycetaceae.g_Blastopirellula
k_Bacteria.p_Firmicutes.c_Bacilli.o_Lactobacillales.f_Enterococcaceae.g_Enterococcus
k_Archaea.p_Euryarchaeota.c_Halobacteria.o_Haloferacales.f_Haloferacaceae.g_Halogeometricum
k_Bacteria.p_Firmicutes.c_Clostridia.o_Thermoanaerobacterales.f_Thermoanaerobacteraceae.g_Caldanaerobacter
k_Bacteria.p_Bacteroidetes.c_Cytophagia.o_Cytophagales.f_Cytophagaceae.g_Flectobacillus
k_Bacteria.p_Proteobacteria.c_Gammaproteobacteria.o_Enterobacteriales.f_Enterobacteriaceae.g_Cosenzaea
k_Bacteria.p_Actinobacteria.c_Actinobacteria.o_Pseudonocardiales.f_Pseudonocardiaceae.g_Allokutzneria
k_Bacteria.p_Firmicutes.c_Clostridia.o_Clostridiales.f_Clostridiaceae.g_Oxobacter
k_Bacteria.p_Actinobacteria.c_Actinobacteria.o_Micromonosporales.f_Micromonosporaceae.g_Actinoplanes
k_Bacteria.p_Bacteroidetes.c_Bacteroidia.o_Bacteroidales.f_Prolixibacteraceae.g_Sunxiuqinia
k_Bacteria.p_Cyanobacteria.o_Oscillatoriales.g_Crinalium
k_Bacteria.p_Firmicutes.c_Negativicutes.o_Selenomonadales.f_Selenomonadaceae.g_Mitsuokella
k_Bacteria.p_Cyanobacteria.o_Nostocales.f_Nostocaceae.g_Aphanizomenon
k_Bacteria.p_Cyanobacteria.o_Stigonematales.g_Chlorogloeopsis
k_Bacteria.p_Firmicutes.c_Bacilli.o_Lactobacillales.f_Enterococcaceae.g_Vagococcus
k_Bacteria.p_Firmicutes.c_Clostridia.o_Clostridiales.f_Lachnospiraceae.g_Coprococcus
k_Bacteria.p_Actinobacteria.c_Actinobacteria.o_Propionibacteriales.f_Nocardioidaceae.g_Nocardioides
k_Bacteria.p_Chlamydiae.c_Chlamydiia.o_Chlamydiales.f_Criblamydiaceae.g_Criblamydia
k_Bacteria.p_Proteobacteria.c_Alphaproteobacteria.o_Rhizobiales.f_Rhodobiaceae.g_Amorphus
k_Bacteria.p_Proteobacteria.c_Deltaproteobacteria.o_Syntrophobacterales.f_Syntrophobacteraceae.g_Syntrophobacter
k_Bacteria.p_Actinobacteria.c_Actinobacteria.o_Micrococcales.f_Intrasporangiaceae.g_Arsenicicoccus
k_Bacteria.p_Proteobacteria.c_Alphaproteobacteria.o_Rhizobiales.f_Rhizobiaceae.g_Ensifer
k_Bacteria.p_Proteobacteria.c_Betaproteobacteria.o_Burkholderiales.g_Leptothrix
k_Bacteria.p_Proteobacteria.c_Alphaproteobacteria.o_Rhizobiales.f_Phyllobacteriaceae.g_Aminobacter
k_Bacteria.p_Proteobacteria.c_Gammaproteobacteria.o_Enterobacteriales.f_Enterobacteriaceae.g_Raoultella
k_Bacteria.p_Actinobacteria.c_Actinobacteria.o_Micrococcales.f_Micrococcaceae.g_Kocuria
k_Bacteria.p_Actinobacteria.c_Actinobacteria.o_Propionibacteriales.f_Propionibacteriaceae.g_Aestuariimicrobium
k_Bacteria.p_Proteobacteria.c_Deltaproteobacteria.o_Desulfurellales.f_Desulfurellaceae.g_Hippea
k_Bacteria.p_Proteobacteria.c_Alphaproteobacteria.o_Rhodospirillales.f_Rhodospirillaceae.g_Fodinicurvata
k_Bacteria.p_Proteobacteria.c_Betaproteobacteria.o_Burkholderiales.f_Oxalobacteraceae.g_Pseudoduganella
k_Archaea.p_Crenarchaeota.c_Thermoprotei.o_Sulfolobales.f_Sulfolobaceae.g_SulfolobuS
k_Bacteria.p_Proteobacteria.c_Betaproteobacteria.o_Burkholderiales.f_Alcaligenaceae.g_Advenella
k_Bacteria.p_Firmicutes.c_Clostridia.o_Clostridiales.f_Clostridiales_Family_XVII._Incertae_Sedis.g_Thermaerobacter
k_Bacteria.p_Bacteroidetes.c_Flavobacteriia.o_Flavobacteriales.f_Flavobacteriaceae.g_Galbibacter
k_Bacteria.p_Actinobacteria.c_Actinobacteria.o_Micrococcales.f_Intrasporangiaceae.g_Serinicoccus
k_Bacteria.p_Bacteroidetes.c_Flavobacteriia.o_Flavobacteriales.f_Flavobacteriaceae.g_Chryseobacterium
k_Bacteria.p_Firmicutes.c_Clostridia.o_Clostridiales.f_Ruminococcaceae.g_Acetivibrio
k_Bacteria.p_Firmicutes.c_Clostridia.o_Thermoanaerobacterales.f_Thermoanaerobacterales_Family_III._Incertae_Sedis.g_Caldicellulosiruptor
k_Bacteria.p_Proteobacteria.c_Gammaproteobacteria.o_Thiotrichales.f_Thiotrichaceae.g_Thiothrix
k_Bacteria.p_Firmicutes.c_Clostridia.o_Clostridiales.f_Clostridiaceae.g_Caloramator
k_Bacteria.p_Proteobacteria.c_Alphaproteobacteria.o_Rhizobiales.f_Rhodobiaceae.g_Tepidicaulis
k_Bacteria.p_Proteobacteria.c_Alphaproteobacteria.o_Rhizobiales.f_Xanthobacteraceae.g_Pseudolabrys
k_Bacteria.p_Aquificae.c_Aquificae.o_Aquificales.f_Aquificaceae.g_Thermocrinis
k_Bacteria.p_Proteobacteria.c_Gammaproteobacteria.o_Chromatiales.f_Ectothiorhodospiraceae.g_Arhodomonas
k_Bacteria.p_Proteobacteria.c_Epsilonproteobacteria.o_Nautiliales.f_Nautiliaceae.g_Lebetimonas
k_Bacteria.p_Firmicutes.c_Clostridia.o_Clostridiales.f_Peptococcaceae.g_Desulfitibacter
k_Archaea.p_Euryarchaeota.c_Thermoplasmata.o_Thermoplasmatales.f_Thermoplasmataceae.g_Thermoplasma
k_Bacteria.p_Actinobacteria.c_Actinobacteria.o_Micrococcales.f_Jonesiaceae.g_Jonesia
k_Bacteria.p_Actinobacteria.c_Actinobacteria.o_Micrococcales.f_Dermacoccaceae.g_Demetria
k_Bacteria.p_Actinobacteria.c_Actinobacteria.o_Nakamurellales.f_Nakamurellaceae.g_Nakamurella
k_Bacteria.p_Bacteroidetes.c_Cytophagia.o_Cytophagales.f_Hymenobacteraceae.g_Hymenobacter
k_Bacteria.p_Firmicutes.c_Bacilli.o_Bacillales.f_Bacillaceae.g_Virgibacillus
k_Bacteria.p_Verrucomicrobia.c_Spartobacteria.g_Candidatus_Xiphinematobacter
k_Bacteria.p_Fibrobacteres.c_Chitinivibrionia.o_Chitinivibrionales.f_Chitinivibrionaceae.g_Chitinivibrio
k_Bacteria.p_Actinobacteria.c_Actinobacteria.o_Streptosporangiales.f_Nocardiopsaceae.g_Nocardiopsis
k_Bacteria.p_Firmicutes.c_Clostridia.o_Clostridiales.f_Lachnospiraceae.g_Johnsonella
k_Bacteria.p_Actinobacteria.c_Actinobacteria.o_Actinopolysporales.f_Actinopolysporaceae.g_Actinopolyspora
k_Bacteria.p_Proteobacteria.c_Gammaproteobacteria.o_Methylococcales.f_Methylococcaceae.g_Methylovulum
k_Archaea.p_Euryarchaeota.c_Methanomicrobia.o_Methanomicrobiales.f_Methanomicrobiaceae.g_Methanofollis
k_Bacteria.p_Proteobacteria.c_Alphaproteobacteria.o_Rhizobiales.f_Phyllobacteriaceae.g_Phyllobacterium
k_Bacteria.p_Bacteroidetes.c_Flavobacteriia.o_Flavobacteriales.f_Blattabacteriaceae.g_Blattabacterium
k_Bacteria.p_Actinobacteria.c_Actinobacteria.o_Geodermatophilales.f_Geodermatophilaceae.g_Blastococcus
k_Bacteria.p_Elusimicrobia.c_Endomicrobia.o_Endomicrobiales.f_Endomicrobiaceae.g_Endomicrobium
k_Bacteria.p_Cyanobacteria.o_Nostocales.f_Nostocaceae.g_Anabaena
k_Bacteria.p_Proteobacteria.c_Alphaproteobacteria.o_Rhodobacterales.f_Rhodobacteraceae.g_Thalassobacter
k_Bacteria.p_Proteobacteria.c_Alphaproteobacteria.o_Rhizobiales.f_Rhizobiaceae.g_Kaistia
k_Bacteria.p_Proteobacteria.c_Alphaproteobacteria.o_Rhizobiales.f_Bradyrhizobiaceae.g_Nitrobacter
k_Bacteria.p_Proteobacteria.c_Betaproteobacteria.o_Burkholderiales.f_Comamonadaceae.g_Alicycliphilus
k_Bacteria.p_Bacteroidetes.c_Sphingobacteriia.o_Sphingobacteriales.f_Sphingobacteriaceae.g_Sphingobacterium
k_Bacteria.p_Bacteroidetes.c_Sphingobacteriia.o_Sphingobacteriales.f_Sphingobacteriaceae.g_Pseudopedobacter
k_Bacteria.p_Proteobacteria.c_Gammaproteobacteria.o_Alteromonadales.f_Alteromonadaceae.g_Catenovulum
k_Bacteria.p_Proteobacteria.c_Alphaproteobacteria.o_Rickettsiales.f_Holosporaceae.g_Holospora
k_Bacteria.p_Bacteroidetes.c_Cytophagia.o_Cytophagales.f_Persicobacteraceae.g_Persicobacter
k_Bacteria.p_Proteobacteria.c_Gammaproteobacteria.o_Methylococcales.f_Methylothermaceae.g_Methylohalobius
k_Bacteria.p_Proteobacteria.c_Deltaproteobacteria.o_Desulfobacterales.f_Desulfobacteraceae.g_Desulfatiglans
k_Bacteria.p_Actinobacteria.c_Actinobacteria.o_Corynebacteriales.g_Hoyosella
k_Bacteria.p_Proteobacteria.c_Deltaproteobacteria.o_Desulfovibrionales.f_Desulfohalobiaceae.g_Desulfonatronovibrio
k_Bacteria.p_Proteobacteria.c_Betaproteobacteria.o_Neisseriales.f_Neisseriaceae.g_Conchiformibius
k_Bacteria.p_Proteobacteria.c_Gammaproteobacteria.o_Pseudomonadales.f_Pseudomonadaceae.g_Pseudomonas
k_Bacteria.p_Firmicutes.c_Clostridia.o_Natranaerobiales.f_Natranaerobiaceae.g_Natranaerobius
k_Bacteria.p_Actinobacteria.c_Actinobacteria.o_Micrococcales.f_Micrococcaceae.g_Paenarthrobacter
k_Bacteria.p_Proteobacteria.c_Betaproteobacteria.o_Neisseriales.f_Neisseriaceae.g_Morococcus
k_Bacteria.p_Actinobacteria.c_Actinobacteria.o_Corynebacteriales.f_Corynebacteriaceae.g_Corynebacterium
k_Bacteria.p_Actinobacteria.c_Actinobacteria.o_Micrococcales.f_Intrasporangiaceae.g_Terrabacter
k_Bacteria.p_Proteobacteria.c_Alphaproteobacteria.o_Rhizobiales.f_Rhizobiaceae.g_Shinella
k_Bacteria.p_Firmicutes.c_Clostridia.o_Clostridiales.f_Clostridiaceae.g_Youngiibacter
k_Archaea.p_Euryarchaeota.c_Archaeoglobi.o_Archaeoglobales.f_Archaeoglobaceae.g_Geoglobus
k_Bacteria.p_Proteobacteria.c_Alphaproteobacteria.o_Sphingomonadales.f_Sphingomonadaceae.g_Sandarakinorhabdus
k_Bacteria.p_Actinobacteria.c_Actinobacteria.o_Micrococcales.f_Promicromonosporaceae.g_Cellulosimicrobium
k_Bacteria.p_Bacteroidetes.c_Bacteroidia.o_Bacteroidales.f_Prevotellaceae.g_Prevotella
k_Bacteria.p_Bacteroidetes.c_Flavobacteriia.o_Flavobacteriales.f_Flavobacteriaceae.g_Mesonia
k_Bacteria.p_Proteobacteria.c_Gammaproteobacteria.o_Enterobacteriales.f_Enterobacteriaceae.g_Leclercia
k_Bacteria.p_Proteobacteria.c_Alphaproteobacteria.o_Rhodospirillales.f_Acetobacteraceae.g_Belnapia
k_Bacteria.p_Actinobacteria.c_Actinobacteria.o_Streptosporangiales.f_Thermomonosporaceae.g_Thermomonospora
k_Bacteria.p_Proteobacteria.c_Alphaproteobacteria.o_Sneathiellales.f_Sneathiellaceae.g_Sneathiella
k_Bacteria.p_Chloroflexi.c_Anaerolineae.o_Anaerolineales.f_Anaerolineaceae.g_Longilinea
k_Bacteria.p_Proteobacteria.c_Gammaproteobacteria.o_Enterobacteriales.f_Enterobacteriaceae.g_Rahnella
k_Bacteria.p_Bacteroidetes.c_Flavobacteriia.o_Flavobacteriales.f_Flavobacteriaceae.g_Ulvibacter
k_Bacteria.p_Proteobacteria.c_Betaproteobacteria.o_Burkholderiales.f_Comamonadaceae.g_Comamonas
k_Bacteria.p_Chloroflexi.c_Dehalococcoidia.o_Dehalococcoidales.f_Dehalococcoidaceae.g_Dehalococcoides
k_Bacteria.p_Firmicutes.c_Bacilli.o_Bacillales.f_Bacillaceae.g_Pontibacillus
k_Bacteria.p_Proteobacteria.c_Gammaproteobacteria.o_Pasteurellales.f_Pasteurellaceae.g_Basfia
k_Bacteria.p_Cyanobacteria.o_Chroococcales.g_Candidatus_Atelocyanobacterium
k_Bacteria.p_Proteobacteria.c_Gammaproteobacteria.o_Thiotrichales.f_Thiotrichaceae.g_Leucothrix
k_Bacteria.p_Proteobacteria.c_Alphaproteobacteria.o_Rhizobiales.f_Methylobacteriaceae.g_Meganema
k_Bacteria.p_Bacteroidetes.c_Chitinophagia.o_Chitinophagales.f_Saprospiraceae.g_Haliscomenobacter
k_Bacteria.p_Rhodothermaeota.c_Balneolia.o_Balneolales.f_Balneolaceae.g_Balneola
k_Bacteria.p_Firmicutes.c_Bacilli.o_Bacillales.f_Paenibacillaceae.g_Paenibacillus
k_Bacteria.p_Proteobacteria.c_Gammaproteobacteria.o_Chromatiales.f_Chromatiaceae.g_Lamprocystis
k_Archaea.p_Euryarchaeota.c_Methanomicrobia.o_Methanosarcinales.f_Methanosaetaceae.g_Methanosaeta
k_Bacteria.p_Proteobacteria.c_Gammaproteobacteria.o_Enterobacteriales.f_Enterobacteriaceae.g_Candidatus_Ishikawaella
k_Bacteria.p_Proteobacteria.c_Gammaproteobacteria.o_Chromatiales.f_Chromatiaceae.g_Thioflavicoccus
k_Bacteria.p_Proteobacteria.c_Deltaproteobacteria.o_Desulfobacterales.f_Desulfobacteraceae.g_Desulfobacter
k_Bacteria.p_Chloroflexi.c_Thermomicrobia.g_Thermorudis
k_Bacteria.p_Firmicutes.c_Clostridia.o_Clostridiales.f_Clostridiaceae.g_Butyricicoccus
k_Bacteria.p_Firmicutes.c_Clostridia.o_Clostridiales.f_Clostridiaceae.g_Alkaliphilus
k_Bacteria.p_Proteobacteria.c_Alphaproteobacteria.o_Rhodobacterales.f_Rhodobacteraceae.g_Marinovum
k_Bacteria.p_Gemmatimonadetes.c_Gemmatimonadetes.o_Gemmatimonadales.f_Gemmatimonadaceae.g_Gemmatirosa
k_Bacteria.p_Proteobacteria.c_Gammaproteobacteria.o_Xanthomonadales.f_Rhodanobacteraceae.g_Oleiagrimonas
k_Bacteria.p_Firmicutes.c_Erysipelotrichia.o_Erysipelotrichales.f_Erysipelotrichaceae.g_Solobacterium
k_Bacteria.p_Actinobacteria.c_Thermoleophilia.o_Solirubrobacterales.f_Solirubrobacteraceae.g_Solirubrobacter
k_Bacteria.p_Synergistetes.c_Synergistia.o_Synergistales.f_Synergistaceae.g_Dethiosulfovibrio
k_Bacteria.p_Proteobacteria.c_Alphaproteobacteria.o_Rhodobacterales.f_Rhodobacteraceae.g_Loktanella
k_Bacteria.p_Proteobacteria.c_Alphaproteobacteria.o_Caulobacterales.f_Caulobacteraceae.g_Asticcacaulis
k_Bacteria.p_Firmicutes.c_Clostridia.o_Clostridiales.f_Peptostreptococcaceae.g_Paeniclostridium
k_Bacteria.p_Firmicutes.c_Clostridia.o_Clostridiales.g_Flavonifractor
k_Bacteria.p_Actinobacteria.c_Actinobacteria.o_Micrococcales.f_Microbacteriaceae.g_Mycetocola
k_Bacteria.p_Proteobacteria.c_Betaproteobacteria.o_Burkholderiales.g_Rubrivivax
k_Bacteria.p_Firmicutes.c_Bacilli.o_Lactobacillales.f_Enterococcaceae.g_Melissococcus
k_Bacteria.p_Proteobacteria.c_Deltaproteobacteria.o_Myxococcales.f_Myxococcaceae.g_Corallococcus
k_Bacteria.p_Firmicutes.c_Clostridia.o_Thermoanaerobacterales.f_Thermoanaerobacteraceae.g_Caldanaerobius
k_Bacteria.p_Actinobacteria.c_Coriobacteriia.o_Eggerthellales.f_Eggerthellaceae.g_Gordonibacter
k_Bacteria.p_Proteobacteria.c_Deltaproteobacteria.o_Desulfarculales.f_Desulfarculaceae.g_Desulfarculus
k_Bacteria.p_Firmicutes.c_Clostridia.o_Thermoanaerobacterales.f_Thermoanaerobacteraceae.g_Thermoanaerobacter
k_Bacteria.p_Firmicutes.c_Clostridia.o_Clostridiales.f_Clostridiaceae.g_Anaerosalibacter
k_Bacteria.p_Proteobacteria.c_Gammaproteobacteria.o_Pasteurellales.f_Pasteurellaceae.g_Haemophilus
k_Bacteria.p_Bacteroidetes.c_Bacteroidia.o_Bacteroidales.f_Porphyromonadaceae.g_Proteiniphilum
k_Bacteria.p_Actinobacteria.c_Actinobacteria.o_Micrococcales.f_Cellulomonadaceae.g_Oerskovia
k_Bacteria.p_Proteobacteria.c_Gammaproteobacteria.o_Oceanospirillales.f_Oceanospirillaceae.g_Thalassolituus
k_Bacteria.p_Deferribacteres.c_Deferribacteres.o_Deferribacterales.f_Deferribacteraceae.g_Mucispirillum
k_Bacteria.p_Firmicutes.c_Erysipelotrichia.o_Erysipelotrichales.f_Erysipelotrichaceae.g_Allobaculum
k_Bacteria.p_Proteobacteria.c_Betaproteobacteria.o_Burkholderiales.f_Alcaligenaceae.g_Taylorella
k_Bacteria.p_Actinobacteria.c_Coriobacteriia.o_Coriobacteriales.f_Coriobacteriaceae.g_Coriobacterium
k_Archaea.p_Euryarchaeota.c_Methanomicrobia.o_Methanosarcinales.f_Methanosarcinaceae.g_Methanolobus
k_Bacteria.p_Proteobacteria.c_Gammaproteobacteria.o_Oceanospirillales.f_Oleiphilaceae.g_Oleiphilus
k_Bacteria.p_Proteobacteria.c_Gammaproteobacteria.o_Oceanospirillales.f_Oceanospirillaceae.g_Amphritea
k_Bacteria.p_Proteobacteria.c_Betaproteobacteria.o_Burkholderiales.f_Comamonadaceae.g_Delftia
k_Bacteria.p_Proteobacteria.c_Gammaproteobacteria.o_Cellvibrionales.f_Cellvibrionaceae.g_Marinagarivorans
k_Bacteria.p_Acidobacteria.g_Thermoanaerobaculum
k_Bacteria.p_Proteobacteria.c_Betaproteobacteria.o_Burkholderiales.f_Alcaligenaceae.g_Alcaligenes
k_Bacteria.p_Firmicutes.c_Erysipelotrichia.o_Erysipelotrichales.f_Erysipelotrichaceae.g_Bulleidia
k_Bacteria.p_Synergistetes.c_Synergistia.o_Synergistales.f_Synergistaceae.g_Aminobacterium
k_Archaea.p_Euryarchaeota.c_Halobacteria.o_Haloferacales.f_Haloferacaceae.g_Haloplanus
k_Bacteria.p_Firmicutes.c_Erysipelotrichia.o_Erysipelotrichales.f_Erysipelotrichaceae.g_Faecalibaculum
k_Bacteria.p_Proteobacteria.c_Gammaproteobacteria.o_Vibrionales.f_Vibrionaceae.g_Alitvibrio
k_Bacteria.p_Actinobacteria.c_Actinobacteria.o_Micrococcales.f_Demequinaceae.g_Demequina
k_Bacteria.p_Bacteroidetes.c_Flavobacteriia.o_Flavobacteriales.f_Flavobacteriaceae.g_Flagellimonas
k_Bacteria.p_Actinobacteria.c_Actinobacteria.o_Pseudonocardiales.f_Pseudonocardiaceae.g_Amycolatopsis
k_Bacteria.p_Proteobacteria.c_Alphaproteobacteria.o_Rhodobacterales.f_Rhodobacteraceae.g_Paracoccus
k_Bacteria.p_Bacteroidetes.c_Sphingobacteriia.o_Sphingobacteriales.f_Sphingobacteriaceae.g_Solitalea
k_Bacteria.p_Fusobacteria.c_Fusobacteriia.o_Fusobacteriales.f_Fusobacteriaceae.g_Psychrilyobacter
k_Archaea.p_Euryarchaeota.c_Thermoplasmata.o_Thermoplasmatales.f_Ferroplasmaceae.g_Ferroplasma
k_Bacteria.p_Proteobacteria.c_Alphaproteobacteria.o_Rickettsiales.f_Rickettsiaceae.g_Orientia
k_Bacteria.p_Proteobacteria.c_Gammaproteobacteria.o_Vibrionales.f_Vibrionaceae.g_Enterovibrio
k_Bacteria.p_Proteobacteria.c_Gammaproteobacteria.o_Enterobacteriales.f_Enterobacteriaceae.g_Franconibacter
k_Bacteria.p_Proteobacteria.c_Gammaproteobacteria.o_Methylococcales.f_Methylococcaceae.g_Methylocaldum
k_Bacteria.p_Proteobacteria.c_Gammaproteobacteria.o_Cellvibrionales.f_Cellvibrionaceae.g_Gilvimarinus
k_Bacteria.p_Proteobacteria.c_Gammaproteobacteria.o_Chromatiales.f_Chromatiaceae.g_Thiocystis
k_Bacteria.p_Firmicutes.c_Bacilli.o_Bacillales.f_Bacillaceae.g_Lentibacillus
k_Bacteria.p_Cyanobacteria.o_Pleurocapsales.g_Pleurocapsa
k_Bacteria.p_Proteobacteria.c_Gammaproteobacteria.o_Alteromonadales.f_Pseudoalteromonadaceae.g_Algicola
k_Archaea.p_Euryarchaeota.c_Methanomicrobia.o_Methanomicrobiales.f_Methanoregulaceae.g_Methanosphaerula
k_Bacteria.p_Actinobacteria.c_Actinobacteria.o_Corynebacteriales.g_Tomitella
k_Bacteria.p_Cyanobacteria.o_Oscillatoriales.g_Planktothricoides
k_Bacteria.p_Proteobacteria.c_Betaproteobacteria.o_Rhodocyclales.f_Rhodocyclaceae.g_Dechloromonas
k_Bacteria.p_Verrucomicrobia.c_Spartobacteria.o_Chthoniobacterales.f_Chthoniobacteraceae.g_Chthoniobacter
k_Bacteria.p_Bacteroidetes.c_Cytophagia.o_Cytophagales.f_Cyclobacteriaceae.g_Mariniradius
k_Bacteria.p_Actinobacteria.c_Actinobacteria.o_Micrococcales.f_Beutenbergiaceae.g_Beutenbergia
k_Archaea.p_Crenarchaeota.c_Thermoprotei.o_Thermoproteales.f_Thermoproteaceae.g_Vulcanisaeta
k_Bacteria.p_Proteobacteria.c_Deltaproteobacteria.o_Desulfovibrionales.f_Desulfomicrobiaceae.g_Desulfomicrobium
k_Bacteria.p_Deinococcus.Thermus.c_Deinococci.o_Thermales.f_Thermaceae.g_Marinithermus
k_Bacteria.p_Firmicutes.c_Clostridia.o_Thermoanaerobacterales.f_Thermoanaerobacterales_Family_IV._Incertae_Sedis.g_Mahella
k_Bacteria.p_Proteobacteria.c_Deltaproteobacteria.o_Desulfarculales.f_Desulfarculaceae.g_Dethiosulfatarculus
k_Bacteria.p_Proteobacteria.c_Gammaproteobacteria.o_Cellvibrionales.f_Spongiibacteraceae.g_Zhongshania
k_Bacteria.p_Actinobacteria.c_Actinobacteria.o_Micromonosporales.f_Micromonosporaceae.g_Catenuloplanes
k_Bacteria.p_Proteobacteria.c_Gammaproteobacteria.o_Pasteurellales.f_Pasteurellaceae.g_Necropsobacter
k_Bacteria.p_Cyanobacteria.o_Stigonematales.g_Hapalosiphon
k_Bacteria.p_Firmicutes.c_Bacilli.o_Lactobacillales.f_Leuconostocaceae.g_Oenococcus
k_Bacteria.p_Proteobacteria.c_Alphaproteobacteria.o_Rhodospirillales.f_Rhodospirillaceae.g_Phaeospirillum
k_Bacteria.p_Proteobacteria.c_Alphaproteobacteria.g_Polymorphum
k_Bacteria.p_Firmicutes.c_Clostridia.o_Clostridiales.f_Lachnospiraceae.g_Marvinbryantia
k_Archaea.p_Euryarchaeota.c_Halobacteria.o_Halobacteriales.f_Halobacteriaceae.g_Halococcus
k_Bacteria.p_Bacteroidetes.o_Bacteroidetes_Order_II._Incertae_sedis.f_Rhodothermaceae.g_Salisaeta
k_Bacteria.p_Proteobacteria.c_Betaproteobacteria.o_Burkholderiales.f_Burkholderiaceae.g_Lautropia
k_Bacteria.p_Proteobacteria.c_Gammaproteobacteria.o_Oceanospirillales.f_Oceanospirillaceae.g_Bermanella
k_Bacteria.p_Proteobacteria.c_Alphaproteobacteria.o_Rhizobiales.f_Methylocystaceae.g_Pleomorphomonas
k_Bacteria.p_Proteobacteria.c_Alphaproteobacteria.o_Rhodobacterales.f_Rhodobacteraceae.g_Pseudovibrio
k_Bacteria.p_Proteobacteria.c_Alphaproteobacteria.o_Rhizobiales.f_Brucellaceae.g_Brucella
k_Bacteria.p_Nitrospirae.c_Nitrospira.o_Nitrospirales.f_Nitrospiraceae.g_Thermodesulfovibrio
k_Bacteria.p_Verrucomicrobia.c_Opitutae.o_Opitutales.f_Opitutaceae.g_Cephaloticoccus
k_Bacteria.p_Proteobacteria.c_Gammaproteobacteria.o_Pseudomonadales.f_Moraxellaceae.g_Alkanindiges
k_Bacteria.p_Proteobacteria.c_Deltaproteobacteria.o_Desulfovibrionales.f_Desulfovibrionaceae.g_Desulfocurvus
k_Bacteria.p_Firmicutes.c_Bacilli.o_Lactobacillales.f_Streptococcaceae.g_Streptococcus
k_Bacteria.p_Actinobacteria.c_Acidimicrobiia.o_Acidimicrobiales.f_Acidimicrobiaceae.g_Acidimicrobium
k_Bacteria.p_Actinobacteria.c_Actinobacteria.o_Frankiales.f_Sporichthyaceae.g_Sporichthya
k_Bacteria.p_Firmicutes.c_Bacilli.o_Bacillales.f_Thermoactinomycetaceae.g_Thermoactinomyces
k_Bacteria.p_Bacteroidetes.c_Cytophagia.o_Cytophagales.f_Cytophagaceae.g_Rudanella
k_Bacteria.p_Aquificae.c_Aquificae.o_Desulfurobacteriales.f_Desulfurobacteriaceae.g_Desulfurobacterium
k_Bacteria.p_Bacteroidetes.c_Flavobacteriia.o_Flavobacteriales.f_Flavobacteriaceae.g_Jejuia
k_Bacteria.p_Firmicutes.c_Bacilli.o_Lactobacillales.f_Carnobacteriaceae.g_Alkalibacterium
k_Bacteria.p_Firmicutes.c_Clostridia.o_Thermoanaerobacterales.f_Thermoanaerobacterales_Family_III._Incertae_Sedis.g_Thermosediminibacter
k_Bacteria.p_Cyanobacteria.o_Oscillatoriales.g_Microcoleus
k_Bacteria.p_Proteobacteria.c_Alphaproteobacteria.o_Rhizobiales.f_Rhodobiaceae.g_Afifella
k_Bacteria.p_Proteobacteria.c_Betaproteobacteria.o_Burkholderiales.f_Alcaligenaceae.g_Pelistega
k_Bacteria.p_Proteobacteria.c_Betaproteobacteria.o_Burkholderiales.f_Oxalobacteraceae.g_Janthinobacterium
k_Bacteria.p_Chloroflexi.c_Anaerolineae.o_Anaerolineales.f_Anaerolineaceae.g_Levilinea
k_Bacteria.p_Firmicutes.c_Bacilli.o_Bacillales.f_Planococcaceae.g_Rummeliibacillus
k_Bacteria.p_Nitrospinae.c_Nitrospinia.o_Nitrospinales.f_Nitrospinaceae.g_Nitrospina
k_Bacteria.p_Cyanobacteria.o_Chroococcales.g_Gloeocapsa
k_Bacteria.p_Firmicutes.c_Bacilli.o_Lactobacillales.f_Enterococcaceae.g_Catellicoccus
k_Bacteria.p_Actinobacteria.c_Actinobacteria.o_Micrococcales.f_Cellulomonadaceae.g_Cellulomonas
k_Bacteria.p_Actinobacteria.c_Actinobacteria.o_Micrococcales.f_Promicromonosporaceae.g_Xylanimonas
k_Bacteria.p_Cyanobacteria.o_Chroococcales.g_Cyanobium
k_Archaea.p_Euryarchaeota.c_Methanobacteria.o_Methanobacteriales.f_Methanothermaceae.g_Methanothermus
k_Bacteria.p_Proteobacteria.c_Alphaproteobacteria.o_Parvularculales.f_Parvularculaceae.g_Parvularcula
k_Bacteria.p_Actinobacteria.c_Actinobacteria.o_Actinomycetales.f_Actinomycetaceae.g_Actinotignum
k_Bacteria.p_Bacteroidetes.c_Bacteroidia.o_Bacteroidales.f_Marinifilaceae.g_Marinifilum
k_Bacteria.p_Proteobacteria.c_Betaproteobacteria.o_Burkholderiales.f_Comamonadaceae.g_Rhodoferax
k_Bacteria.p_Actinobacteria.c_Actinobacteria.o_Micromonosporales.f_Micromonosporaceae.g_Catelliglobosispora
k_Bacteria.p_Proteobacteria.c_Gammaproteobacteria.o_Chromatiales.f_Chromatiaceae.g_Nitrosococcus
k_Bacteria.p_Proteobacteria.c_Alphaproteobacteria.o_Rhodobacterales.f_Rhodobacteraceae.g_Salipiger
k_Bacteria.p_Proteobacteria.c_Alphaproteobacteria.o_Rhizobiales.f_Beijerinckiaceae.g_Methyloferula
k_Bacteria.p_Proteobacteria.c_Betaproteobacteria.o_Neisseriales.f_Chromobacteriaceae.g_Gulbenkiania
k_Bacteria.p_Firmicutes.c_Clostridia.o_Clostridiales.f_Peptostreptococcaceae.g_Terrisporobacter
k_Bacteria.p_Proteobacteria.c_Alphaproteobacteria.o_Rhodobacterales.f_Rhodobacteraceae.g_Octadecabacter
k_Bacteria.p_Proteobacteria.c_Deltaproteobacteria.o_Desulfobacterales.f_Desulfobulbaceae.g_Desulfurivibrio
k_Bacteria.p_Proteobacteria.c_Gammaproteobacteria.o_Pseudomonadales.f_Moraxellaceae.g_Psychrobacter
k_Bacteria.p_Proteobacteria.c_Alphaproteobacteria.o_Rhodobacterales.f_Rhodobacteraceae.g_Jannaschia
k_Archaea.p_Euryarchaeota.c_Archaeoglobi.o_Archaeoglobales.f_Archaeoglobaceae.g_Archaeoglobus
k_Bacteria.p_Bacteroidetes.c_Flavobacteriia.o_Flavobacteriales.f_Flavobacteriaceae.g_Siansivirga
k_Bacteria.p_Firmicutes.c_Clostridia.o_Halanaerobiales.f_Halanaerobiaceae.g_Halothermothrix
k_Bacteria.p_Proteobacteria.c_Alphaproteobacteria.o_Rhodospirillales.f_Acetobacteraceae.g_Gluconacetobacter
k_Bacteria.p_Actinobacteria.c_Actinobacteria.o_Micrococcales.f_Intrasporangiaceae.g_Phycicoccus
k_Bacteria.p_Proteobacteria.c_Deltaproteobacteria.o_Desulfobacterales.f_Desulfobacteraceae.g_Desulfatibacillum
k_Bacteria.p_Firmicutes.c_Bacilli.o_Lactobacillales.f_Carnobacteriaceae.g_Dolosigranulum
k_Archaea.p_Euryarchaeota.c_Halobacteria.o_Halobacteriales.f_Halobacteriaceae.g_Halarchaeum
k_Bacteria.p_Proteobacteria.c_Alphaproteobacteria.o_Rickettsiales.f_Anaplasmataceae.g_Ehrlichia
k_Bacteria.p_Firmicutes.c_Clostridia.o_Clostridiales.f_Caldicoprobacteraceae.g_Caldicoprobacter
k_Bacteria.p_Firmicutes.c_Bacilli.o_Bacillales.f_Bacillaceae.g_Salinibacillus
k_Bacteria.p_Proteobacteria.c_Deltaproteobacteria.o_Desulfurellales.f_Desulfurellaceae.g_Desulfurella
k_Bacteria.p_Firmicutes.c_Clostridia.o_Clostridiales.f_Lachnospiraceae.g_Pseudobutyrivibrio
k_Bacteria.p_Bacteroidetes.c_Flavobacteriia.o_Flavobacteriales.f_Flavobacteriaceae.g_Capnocytophaga
k_Bacteria.p_Actinobacteria.c_Actinobacteria.o_Micrococcales.f_Microbacteriaceae.g_Humibacter
k_Bacteria.p_Proteobacteria.c_Gammaproteobacteria.o_Enterobacteriales.f_Enterobacteriaceae.g_Escherichia
k_Bacteria.p_Proteobacteria.c_Alphaproteobacteria.o_Rhizobiales.f_Aurantimonadaceae.g_Martelella
k_Bacteria.p_Firmicutes.c_Bacilli.o_Bacillales.f_Bacillaceae.g_Lysinibacillus
k_Bacteria.p_Proteobacteria.c_Gammaproteobacteria.o_Cellvibrionales.f_Spongiibacteraceae.g_Spongiibacter
k_Bacteria.p_Proteobacteria.c_Betaproteobacteria.o_Neisseriales.f_Chromobacteriaceae.g_Paludibacterium
k_Bacteria.p_Proteobacteria.c_Gammaproteobacteria.o_Aeromonadales.f_Aeromonadaceae.g_Aeromonas
k_Bacteria.p_Chlamydiae.c_Chlamydiia.o_Chlamydiales.f_Parachlamydiaceae.g_Candidatus_Protochlamydia
k_Bacteria.p_Proteobacteria.c_Betaproteobacteria.o_Burkholderiales.f_Comamonadaceae.g_Variovorax
k_Bacteria.p_Proteobacteria.c_Betaproteobacteria.o_Burkholderiales.f_Burkholderiaceae.g_Cupriavidus
k_Bacteria.p_Bacteroidetes.c_Flavobacteriia.o_Flavobacteriales.f_Flavobacteriaceae.g_Gelidibacter
k_Bacteria.p_Thermotogae.c_Thermotogae.o_Kosmotogales.f_Kosmotogaceae.g_Mesotoga
k_Bacteria.p_Firmicutes.c_Clostridia.o_Clostridiales.f_Lachnospiraceae.g_Cellulosilyticum
k_Bacteria.p_Actinobacteria.c_Actinobacteria.o_Micrococcales.f_Dermabacteraceae.g_Brachybacterium
k_Archaea.p_Euryarchaeota.c_Halobacteria.o_Halobacteriales.f_Halobacteriaceae.g_Hatadaptatus
k_Bacteria.p_Proteobacteria.c_Alphaproteobacteria.o_Rhizobiales.f_Phyllobacteriaceae.g_Hoeflea
k_Bacteria.p_Verrucomicrobia.c_Verrucomicrobiae.o_Verrucomicrobiales.f_Verrucomicrobia_subdivision_3.g_Pedosphaera
k_Bacteria.p_Proteobacteria.c_Alphaproteobacteria.o_Rhodospirillales.f_Acetobacteraceae.g_Commensalibacter
k_Bacteria.p_Firmicutes.c_Clostridia.o_Thermoanaerobacterales.f_Thermodesulfobiaceae.g_Thermodesulfobium
k_Bacteria.p_Proteobacteria.c_Alphaproteobacteria.o_Rickettsiales.f_Candidatus_Paracaedibacteraceae.g_Candidatus_Odyssella
k_Bacteria.p_Proteobacteria.c_Alphaproteobacteria.o_Rhizobiales.f_Bradyrhizobiaceae.g_Bradyrhizobium
k_Bacteria.p_Actinobacteria.c_Actinobacteria.o_Streptosporangiales.f_Streptosporangiaceae.g_Herbidospora
k_Bacteria.p_Proteobacteria.c_Alphaproteobacteria.o_Rhodobacterales.f_Rhodobacteraceae.g_Ketogulonicigenium
k_Archaea.p_Euryarchaeota.c_Methanomicrobia.o_Methanomicrobiales.f_Methanospirillaceae.g_Methanospirillum
k_Bacteria.p_Proteobacteria.c_Alphaproteobacteria.o_Rhodobacterales.f_Hyphomonadaceae.g_Hellea
k_Bacteria.p_Proteobacteria.c_Gammaproteobacteria.o_Chromatiales.f_Chromatiaceae.g_Thiorhodovibrio
k_Bacteria.p_Proteobacteria.c_Gammaproteobacteria.g_Wohlfahrtiimonas
k_Bacteria.p_Proteobacteria.c_Betaproteobacteria.o_Rhodocyclales.f_Rhodocyclaceae.g_Azovibrio
k_Bacteria.p_Proteobacteria.c_Alphaproteobacteria.o_Rhodobacterales.f_Rhodobacteraceae.g_Pannonibacter
k_Bacteria.p_Proteobacteria.c_Alphaproteobacteria.o_Rhizobiales.f_Phyllobacteriaceae.g_Mesorhizobium
k_Bacteria.p_Firmicutes.c_Negativicutes.o_Selenomonadales.f_Sporomusaceae.g_Thermosinus
k_Bacteria.p_Proteobacteria.c_Alphaproteobacteria.o_Rhodobacterales.f_Hyphomonadaceae.g_Hirschia
k_Bacteria.p_Proteobacteria.c_Betaproteobacteria.o_Burkholderiales.f_Comamonadaceae.g_Hylemonella
k_Bacteria.p_Planctomycetes.c_Phycisphaerae.o_Phycisphaerales.f_Phycisphaeraceae.g_Phycisphaera
k_Bacteria.p_Proteobacteria.c_Epsilonproteobacteria.o_Campylobacterales.f_Helicobacteraceae.g_Sulfurimonas
k_Bacteria.p_Firmicutes.c_Erysipelotrichia.o_Erysipelotrichales.f_Erysipelotrichaceae.g_Holdemania
k_Bacteria.p_Proteobacteria.c_Betaproteobacteria.o_Burkholderiales.f_Comamonadaceae.g_Caldimonas
k_Bacteria.p_Proteobacteria.c_Alphaproteobacteria.o_Caulobacterales.f_Caulobacteraceae.g_Caulobacter
k_Bacteria.p_Proteobacteria.c_Gammaproteobacteria.o_Oceanospirillales.f_Halomonadaceae.g_Chromohalobacter
k_Bacteria.p_Proteobacteria.c_Alphaproteobacteria.o_Rhodobacterales.f_Rhodobacteraceae.g_Gemmobacter
k_Bacteria.p_Bacteroidetes.o_Bacteroidetes_Order_II._Incertae_sedis.f_Rhodothermaceae.g_Rhodothermus
k_Bacteria.p_Firmicutes.c_Bacilli.o_Lactobacillales.f_Carnobacteriaceae.g_Granulicatella
k_Bacteria.p_Proteobacteria.c_Gammaproteobacteria.o_Alteromonadales.f_Alteromonadaceae.g_Alteromonas
k_Bacteria.p_Bacteroidetes.c_Flavobacteriia.o_Flavobacteriales.f_Flavobacteriaceae.g_Eudoraea
k_Bacteria.p_Proteobacteria.c_Alphaproteobacteria.o_Sphingomonadales.f_Sphingomonadaceae.g_Sphingorhabdus
k_Bacteria.p_Proteobacteria.c_Alphaproteobacteria.o_Rhodospirillales.f_Rhodospirillaceae.g_Thalassospira
k_Bacteria.p_Cyanobacteria.o_Nostocales.f_Rivulariaceae.g_Rivularia
k_Bacteria.p_Actinobacteria.c_Actinobacteria.o_Pseudonocardiales.f_Pseudonocardiaceae.g_Alloactinosynnema
k_Bacteria.p_Proteobacteria.c_Betaproteobacteria.o_Burkholderiales.f_Comamonadaceae.g_Caenimonas
k_Bacteria.p_Proteobacteria.c_Gammaproteobacteria.o_Oceanospirillales.f_Oceanospirillaceae.g_Balneatrix
k_Bacteria.p_Firmicutes.c_Tissierellia.g_Kallipyga
k_Bacteria.p_Cyanobacteria.o_Nostocales.f_Nostocaceae.g_Nodularia
k_Bacteria.p_Bacteroidetes.c_Flavobacteriia.o_Flavobacteriales.f_Flavobacteriaceae.g_Flavobacterium
k_Bacteria.p_Firmicutes.c_Negativicutes.o_Selenomonadales.f_Sporomusaceae.g_Pelosinus
k_Bacteria.p_Bacteroidetes.c_Bacteroidia.o_Bacteroidales.f_Porphyromonadaceae.g_Coprobacter
k_Bacteria.p_Proteobacteria.c_Alphaproteobacteria.o_Rhodobacterales.f_Rhodobacteraceae.g_Dinoroseobacter
k_Bacteria.p_Proteobacteria.c_Gammaproteobacteria.o_Enterobacteriales.f_Enterobacteriaceae.g_Salmonella
k_Bacteria.p_Proteobacteria.c_Alphaproteobacteria.o_Rhodobacterales.f_Rhodobacteraceae.g_Thioclava
k_Bacteria.p_Proteobacteria.c_Gammaproteobacteria.o_Chromatiales.f_Ectothiorhodospiraceae.g_Halorhodospira
k_Bacteria.p_Proteobacteria.c_Betaproteobacteria.o_Neisseriales.f_Neisseriaceae.g_Eikenella
k_Bacteria.p_Firmicutes.c_Bacilli.o_Bacillales.f_Bacillaceae.g_Halobacillus
k_Bacteria.p_Proteobacteria.c_Gammaproteobacteria.o_Thiotrichales.f_Piscirickettsiaceae.g_Methylophaga
k_Bacteria.p_Bacteroidetes.c_Cytophagia.o_Cytophagales.f_Cytophagaceae.g_Microscilla
k_Bacteria.p_Proteobacteria.c_Betaproteobacteria.o_Burkholderiales.f_Oxalobacteraceae.g_Collimonas
k_Bacteria.p_Firmicutes.c_Bacilli.o_Bacillales.f_Paenibacillaceae.g_Thermobacillus
k_Bacteria.p_Firmicutes.c_Bacilli.o_Bacillales.g_Gemella
k_Bacteria.p_Cyanobacteria.o_Nostocales.f_Microchaetaceae.g_Hassallia
k_Bacteria.p_Acidobacteria.c_Solibacteres.o_Solibacterales.g_Bryobacter
k_Bacteria.p_Proteobacteria.c_Alphaproteobacteria.o_Rhodobacterales.f_Rhodobacteraceae.g_Pseudodonghicola
k_Bacteria.p_Proteobacteria.c_Deltaproteobacteria.o_Myxococcales.f_Cystobacteraceae.g_Cystobacter
k_Bacteria.p_Proteobacteria.c_Alphaproteobacteria.o_Rhodobacterales.f_Rhodobacteraceae.g_Celeribacter
k_Bacteria.p_Firmicutes.c_Negativicutes.o_Selenomonadales.f_Selenomonadaceae.g_Megamonas
k_Bacteria.p_Proteobacteria.c_Betaproteobacteria.o_Burkholderiales.f_Comamonadaceae.g_Verminephrobacter
k_Bacteria.p_Firmicutes.c_Bacilli.o_Bacillales.f_Bacillaceae.g_Salimicrobium
k_Bacteria.p_Actinobacteria.c_Actinobacteria.o_Geodermatophilales.f_Geodermatophilaceae.g_Geodermatophilus
k_Bacteria.p_Actinobacteria.c_Actinobacteria.o_Propionibacteriales.f_Nocardioidaceae.g_Propionicicella
k_Bacteria.p_Proteobacteria.c_Alphaproteobacteria.o_Rhodospirillales.f_Rhodospirillaceae.g_Candidatus_Endolissoclinum
k_Bacteria.p_Proteobacteria.c_Alphaproteobacteria.o_Rhizobiales.f_Xanthobacteraceae.g_Xanthobacter
k_Bacteria.p_Actinobacteria.c_Actinobacteria.o_Propionibacteriales.f_Nocardioidaceae.g_Aeromicrobium
k_Bacteria.p_Proteobacteria.c_Betaproteobacteria.o_Methylophilales.f_Methylophilaceae.g_Methylophilus
k_Bacteria.p_Bacteroidetes.c_Flavobacteriia.o_Flavobacteriales.f_Flavobacteriaceae.g_Salegentibacter
k_Bacteria.p_Proteobacteria.c_Gammaproteobacteria.o_Enterobacteriales.f_Enterobacteriaceae.g_Klebsiella
k_Bacteria.p_Bacteroidetes.c_Flavobacteriia.o_Flavobacteriales.f_Flavobacteriaceae.g_Apibacter
k_Bacteria.p_Cyanobacteria.o_Nostocales.f_Microchaetaceae.g_Tolypothrix
k_Bacteria.p_Proteobacteria.c_Gammaproteobacteria.o_Salinisphaerales.f_Salinisphaeraceae.g_Salinisphaera
k_Bacteria.p_Proteobacteria.c_Betaproteobacteria.o_Burkholderiales.f_Alcaligenaceae.g_Pusillimonas
k_Bacteria.p_Proteobacteria.c_Epsilonproteobacteria.o_Campylobacterales.f_Helicobacteraceae.g_Sulfuricurvum
k_Bacteria.p_Proteobacteria.c_Betaproteobacteria.o_Neisseriales.f_Chromobacteriaceae.g_Chromobacterium
k_Bacteria.p_Bacteroidetes.c_Chitinophagia.o_Chitinophagales.f_Saprospiraceae.g_Saprospira
k_Bacteria.p_Tenericutes.c_Mollicutes.o_Mycoplasmatales.f_Mycoplasmataceae.g_Mycoplasma
k_Bacteria.p_Actinobacteria.c_Actinobacteria.o_Micromonosporales.f_Micromonosporaceae.g_Hamadaea
k_Bacteria.p_Firmicutes.c_Erysipelotrichia.o_Erysipelotrichales.f_Erysipelotrichaceae.g_Dielma
k_Bacteria.p_Firmicutes.c_Clostridia.o_Clostridiales.f_Peptostreptococcaceae.g_Intestinibacter
k_Bacteria.p_Proteobacteria.c_Gammaproteobacteria.o_Enterobacteriales.f_Enterobacteriaceae.g_Leminorella
k_Bacteria.p_Proteobacteria.c_Gammaproteobacteria.o_Oceanospirillales.f_Hahellaceae.g_Zooshikella
k_Bacteria.p_Proteobacteria.c_Deltaproteobacteria.o_Desulfuromonadales.f_Geobacteraceae.g_Geoalkalibacter
k_Bacteria.p_Fibrobacteres.c_Fibrobacteria.o_Fibrobacterales.f_Fibrobacteraceae.g_Fibrobacter
k_Bacteria.p_Firmicutes.c_Clostridia.o_Clostridiales.f_Clostridiaceae.g_Hungatella
k_Bacteria.p_Proteobacteria.c_Betaproteobacteria.o_Burkholderiales.g_Sphaerotilus
k_Bacteria.p_Chlamydiae.c_Chlamydiia.o_Chlamydiales.f_Chlamydiaceae.g_Chlamydia
k_Bacteria.p_Firmicutes.c_Bacilli.o_Bacillales.f_Bacillaceae.g_Terribacillus
k_Bacteria.p_Proteobacteria.c_Alphaproteobacteria.o_Rhodobacterales.f_Rhodobacteraceae.g_Tateyamaria
k_Bacteria.p_Actinobacteria.c_Actinobacteria.o_Glycomycetales.f_Glycomycetaceae.g_Haloglycomyces
k_Bacteria.p_Firmicutes.c_Bacilli.o_Bacillales.f_Planococcaceae.g_Solibacillus
k_Archaea.p_Euryarchaeota.c_Halobacteria.o_Natrialbales.f_Natrialbaceae.g_Natrinema
k_Bacteria.p_Firmicutes.c_Tissierellia.o_Tissierellales.f_Peptoniphilaceae.g_Peptoniphilus
k_Bacteria.p_Actinobacteria.c_Actinobacteria.o_Pseudonocardiales.f_Pseudonocardiaceae.g_Saccharomonospora
k_Bacteria.p_Proteobacteria.c_Gammaproteobacteria.o_Pseudomonadales.f_Moraxellaceae.g_Perlucidibaca
k_Bacteria.p_Proteobacteria.c_Gammaproteobacteria.o_Chromatiales.f_Chromatiaceae.g_Allochromatium
k_Bacteria.p_Bacteroidetes.c_Flavobacteriia.o_Flavobacteriales.f_Flavobacteriaceae.g_Psychroserpens
k_Bacteria.p_Proteobacteria.c_Epsilonproteobacteria.o_Campylobacterales.g_Nitratifractor
k_Bacteria.p_Cyanobacteria.o_Oscillatoriales.g_Pseudanabaena
k_Bacteria.p_Firmicutes.c_Clostridia.o_Thermoanaerobacterales.f_Thermoanaerobacteraceae.g_Thermacetogenium
k_Bacteria.p_Tenericutes.c_Mollicutes.o_Entomoplasmatales.f_Entomoplasmataceae.g_Entomoplasma
k_Archaea.p_Crenarchaeota.c_Thermoprotei.o_Acidilobales.f_Acidilobaceae.g_AcidilobuS
k_Bacteria.p_Actinobacteria.c_Actinobacteria.o_Corynebacteriales.f_Dietziaceae.g_Dietzia
k_Bacteria.p_Proteobacteria.c_Alphaproteobacteria.o_Rhodospirillales.f_Rhodospirillaceae.g_Skermanella
k_Bacteria.p_Actinobacteria.c_Actinobacteria.o_Micrococcales.f_Intrasporangiaceae.g_Tetrasphaera
k_Bacteria.p_Proteobacteria.c_Gammaproteobacteria.o_Legionellales.f_Coxiellaceae.g_Coxiella
k_Archaea.p_Euryarchaeota.c_Methanomicrobia.o_Methanomicrobiales.f_Methanomicrobiaceae.g_Methanoplanus
k_Bacteria.p_Actinobacteria.c_Nitriliruptoria.o_Nitriliruptorales.f_Nitriliruptoraceae.g_Nitriliruptor
k_Bacteria.p_Proteobacteria.c_Alphaproteobacteria.o_Rickettsiales.f_Anaplasmataceae.g_Wolbachia
k_Archaea.p_Euryarchaeota.c_Methanomicrobia.o_Methanosarcinales.f_Methanosarcinaceae.g_Methanomethylovorans
k_Bacteria.p_Firmicutes.c_Bacilli.o_Bacillales.f_Bacillaceae.g_Anaerobacillus
k_Bacteria.p_Proteobacteria.c_Alphaproteobacteria.o_Rhizobiales.f_Methylocystaceae.g_Methylosinus
k_Bacteria.p_Proteobacteria.c_Alphaproteobacteria.o_Rhodospirillales.f_Acetobacteraceae.g_Acidocella
k_Bacteria.p_Actinobacteria.c_Actinobacteria.o_Geodermatophilales.f_Geodermatophilaceae.g_Modestobacter
k_Bacteria.p_Proteobacteria.c_Betaproteobacteria.o_Rhodocyclales.f_Rhodocyclaceae.g_Azonexus
k_Archaea.p_Euryarchaeota.c_Halobacteria.o_Halobacteriales.f_Halobacteriaceae.g_Halalkalicoccus
k_Bacteria.p_Actinobacteria.c_Actinobacteria.o_Bifidobacteriales.f_Bifidobacteriaceae.g_Scardovia
k_Bacteria.p_Proteobacteria.c_Betaproteobacteria.o_Nitrosomonadales.f_Nitrosomonadaceae.g_Nitrosomonas
k_Bacteria.p_Actinobacteria.c_Actinobacteria.o_Micrococcales.f_Micrococcaceae.g_Rothia
k_Bacteria.p_Actinobacteria.c_Actinobacteria.o_Micrococcales.f_Micrococcaceae.g_Arthrobacter
k_Bacteria.p_Proteobacteria.c_Gammaproteobacteria.o_Cellvibrionales.f_Halieaceae.g_Congregibacter
k_Bacteria.p_Proteobacteria.c_Alphaproteobacteria.o_Rhodospirillales.f_Acetobacteraceae.g_Acetobacter
k_Bacteria.p_Proteobacteria.c_Betaproteobacteria.o_Rhodocyclales.f_Rhodocyclaceae.g_Azospira
k_Bacteria.p_Actinobacteria.c_Actinobacteria.o_Streptomycetales.f_Streptomycetaceae.g_Kitasatospora
k_Bacteria.p_Proteobacteria.c_Alphaproteobacteria.o_Rhodobacterales.f_Rhodobacteraceae.g_Pseudorhodobacter
k_Bacteria.p_Proteobacteria.c_Betaproteobacteria.o_Burkholderiales.f_Oxalobacteraceae.g_Oxalobacter
k_Bacteria.p_Proteobacteria.c_Acidithiobacillia.o_Acidithiobacillales.f_Acidithiobacillaceae.g_Acidithiobacillus
k_Bacteria.p_Proteobacteria.c_Gammaproteobacteria.o_Cellvibrionales.f_Microbulbiferaceae.g_Microbulbifer
k_Bacteria.p_Proteobacteria.c_Alphaproteobacteria.o_Rhodobacterales.f_Rhodobacteraceae.g_Roseibacterium
k_Bacteria.p_Firmicutes.c_Clostridia.o_Clostridiales.f_Peptococcaceae.g_Desulfurispora
k_Bacteria.p_Cyanobacteria.o_Chroococcales.g_Dactylococcopsis
k_Bacteria.p_Proteobacteria.c_Betaproteobacteria.o_Burkholderiales.f_Oxalobacteraceae.g_Herminiimonas
k_Bacteria.p_Dictyoglomi.c_Dictyoglomia.o_Dictyoglomales.f_Dictyoglomaceae.g_Dictyoglomus
k_Bacteria.p_Proteobacteria.c_Gammaproteobacteria.o_Cellvibrionales.f_Cellvibrionaceae.g_Simiduia
k_Bacteria.p_Proteobacteria.c_Alphaproteobacteria.o_Rickettsiales.f_Anaplasmataceae.g_Neorickettsia
k_Bacteria.p_Actinobacteria.c_Actinobacteria.o_Streptosporangiales.f_Thermomonosporaceae.g_Actinomadura
k_Bacteria.p_Proteobacteria.c_Betaproteobacteria.g_Candidatus_Nasuia
k_Bacteria.p_Firmicutes.c_Bacilli.o_Bacillales.f_Bacillaceae.g_Ornithinibacillus
k_Bacteria.p_Bacteroidetes.c_Flavobacteriia.o_Flavobacteriales.f_Flavobacteriaceae.g_Olleya
k_Bacteria.p_Actinobacteria.c_Actinobacteria.o_Pseudonocardiales.f_Pseudonocardiaceae.g_Lechevalieria
k_Bacteria.p_Proteobacteria.c_Deltaproteobacteria.o_Desulfuromonadales.f_Geobacteraceae.g_Geobacter
k_Bacteria.p_Proteobacteria.c_Gammaproteobacteria.o_Enterobacteriales.f_Enterobacteriaceae.g_Eklwardsiella
k_Bacteria.p_Deinococcus.Thermus.c_Deinococci.o_Thermales.f_Thermaceae.g_Oceanithermus
k_Archaea.p_Crenarchaeota.c_Thermoprotei.o_Sulfolobales.f_Sulfolobaceae.g_Metallosphaera
k_Bacteria.p_Bacteroidetes.c_Cytophagia.o_Cytophagales.f_Flammeovirgaceae.g_Marivirga
k_Bacteria.p_Bacteroidetes.c_Flavobacteriia.o_Flavobacteriales.f_Flavobacteriaceae.g_Algibacter
k_Bacteria.p_Proteobacteria.c_Gammaproteobacteria.o_Xanthomonadales.f_Xanthomonadaceae.g_Luteimonas
k_Bacteria.p_Proteobacteria.c_Gammaproteobacteria.o_Cellvibrionales.f_Halieaceae.g_Pseudohaliea
k_Bacteria.p_Proteobacteria.c_Alphaproteobacteria.o_Rhodospirillales.f_Rhodospirillaceae.g_Tistrella
k_Archaea.p_Crenarchaeota.c_Thermoprotei.o_Desulfurococcales.f_Desulfurococcaceae.g_Aeropyrum
k_Bacteria.p_Proteobacteria.c_Betaproteobacteria.o_Neisseriales.f_Chromobacteriaceae.g_Leeia
k_Bacteria.p_Proteobacteria.c_Deltaproteobacteria.o_Desulfobacterales.f_Desulfobacteraceae.g_Desulfotignum
k_Bacteria.p_Actinobacteria.c_Actinobacteria.o_Micrococcales.f_Microbacteriaceae.g_Agreia
k_Bacteria.p_Firmicutes.c_Clostridia.o_Clostridiales.f_Clostridiaceae.g_Proteiniclasticum
k_Bacteria.p_Actinobacteria.c_Actinobacteria.o_Pseudonocardiales.f_Pseudonocardiaceae.g_Lentzea
k_Bacteria.p_Firmicutes.c_Bacilli.o_Bacillales.f_Bacillaceae.g_Psychrobacillus
k_Bacteria.p_Actinobacteria.c_Actinobacteria.o_Propionibacteriales.f_Propionibacteriaceae.g_Propionimicrobium
k_Bacteria.p_Proteobacteria.c_Gammaproteobacteria.o_Vibrionales.f_Vibrionaceae.g_Vibrio
k_Bacteria.p_Planctomycetes.c_Planctomycetia.o_Planctomycetales.f_Isosphaeraceae.g_Singulisphaera
k_Bacteria.p_Firmicutes.c_Bacilli.o_Bacillales.f_Bacillaceae.g_Hydrogenibacillus
k_Bacteria.p_Proteobacteria.c_Betaproteobacteria.o_Methylophilales.f_Methylophilaceae.g_Candidatus_Methylopumilus
k_Bacteria.p_Aquificae.c_Aquificae.o_Aquificales.f_Hydrogenothermaceae.g_Sulfurihydrogenibium
k_Bacteria.p_Thermotogae.c_Thermotogae.o_Petrotogales.g_Petrotoga
k_Bacteria.p_Proteobacteria.c_Gammaproteobacteria.o_Pseudomonadales.f_Pseudomonadaceae.g_Azotobacter
k_Bacteria.p_Proteobacteria.c_Alphaproteobacteria.o_Rhizobiales.f_Methylocystaceae.g_Methylocystis
k_Bacteria.p_Proteobacteria.c_Alphaproteobacteria.g_Geminicoccus
k_Bacteria.p_Actinobacteria.c_Actinobacteria.o_Catenulisporales.f_Actinospicaceae.g_Actinospica
k_Bacteria.p_Acidobacteria.c_Acidobacteriia.o_Acidobacteriales.f_Acidobacteriaceae.g_Granulicella
k_Bacteria.p_Firmicutes.c_Bacilli.o_Bacillales.f_Thermoactinomycetaceae.g_Desmospora
k_Bacteria.p_Bacteroidetes.c_Bacteroidia.o_Bacteroidales.f_Marinilabiliaceae.g_Geofilum
k_Bacteria.p_Actinobacteria.c_Actinobacteria.o_Streptosporangiales.f_Streptosporangiaceae.g_Microtetraspora
k_Bacteria.p_Proteobacteria.c_Deltaproteobacteria.o_Myxococcales.f_Kofleriaceae.g_Haliangium
k_Archaea.p_Candidatus_Korarchaeota.g_Candidatus_Korarchaeum
k_Bacteria.p_Bacteroidetes.c_Bacteroidia.o_Bacteroidales.f_Prevotellaceae.g_Paraprevoteha
k_Bacteria.p_Aquificae.c_Aquificae.o_Aquificales.f_Aquificaceae.g_Hydrogenobaculum
k_Bacteria.p_Proteobacteria.c_Gammaproteobacteria.o_Pasteurellales.f_Pasteurellaceae.g_Pasteurella
k_Bacteria.p_Bacteroidetes.c_Flavobacteriia.o_Flavobacteriales.f_Flavobacteriaceae.g_Tenacibaculum
k_Bacteria.p_Bacteroidetes.c_Chitinophagia.o_Chitinophagales.f_Saprospiraceae.g_Lewinella
k_Bacteria.p_Actinobacteria.c_Actinobacteria.o_Pseudonocardiales.f_Pseudonocardiaceae.g_Saccharothrix
k_Bacteria.p_Proteobacteria.c_Gammaproteobacteria.o_Cellvibrionales.f_Halieaceae.g_Haliea
k_Archaea.p_Euryarchaeota.c_Methanomicrobia.o_Methanosarcinales.f_Methanosarcinaceae.g_Methanococcoides
k_Bacteria.p_Bacteroidetes.c_Chitinophagia.o_Chitinophagales.f_Chitinophagaceae.g_Chitinophaga
k_Bacteria.p_Proteobacteria.c_Betaproteobacteria.o_Neisseriales.f_Neisseriaceae.g_Neisseria
k_Archaea.p_Euryarchaeota.c_Halobacteria.o_Haloferacales.f_Haloferacaceae.g_Haloquadratum
k_Bacteria.p_Proteobacteria.c_Gammaproteobacteria.o_Oceanospirillales.f_Halomonadaceae.g_Halotalea
k_Bacteria.p_Bacteroidetes.c_Bacteroidia.o_Bacteroidales.f_Porphyromonadaceae.g_Paludibacter
k_Bacteria.p_Actinobacteria.c_Actinobacteria.o_Pseudonocardiales.f_Pseudonocardiaceae.g_Saccharopolyspora
k_Bacteria.p_Bacteroidetes.c_Bacteroidia.o_Bacteroidales.f_Prevotellaceae.g_Alloprevotella
k_Bacteria.p_Cyanobacteria.o_Nostocales.f_Nostocaceae.g_Trichormus
k_Bacteria.p_Chlorobi.c_Chlorobia.o_Chlorobiales.f_Chlorobiaceae.g_Pelodictyon
k_Bacteria.p_Synergistetes.c_Synergistia.o_Synergistales.f_Synergistaceae.g_Thermovirga
k_Bacteria.p_Proteobacteria.c_Alphaproteobacteria.o_Rhizobiales.f_Hyphomicrobiaceae.g_Filomicrobium
k_Bacteria.p_Firmicutes.c_Bacilli.o_Bacillales.f_Bacillaceae.g_Halolactibacillus
k_Bacteria.p_Proteobacteria.c_Gammaproteobacteria.o_Alteromonadales.f_Alteromonadaceae.g_Lacimicrobium
k_Bacteria.p_Bacteroidetes.c_Cytophagia.o_Cytophagales.f_Cytophagaceae.g_Dyadobacter
k_Bacteria.p_Bacteroidetes.c_Flavobacteriia.o_Flavobacteriales.f_Flavobacteriaceae.g_Zobellia
k_Bacteria.p_Proteobacteria.c_Alphaproteobacteria.o_Rhodospirillales.f_Rhodospirillaceae.g_Nisaea
k_Bacteria.p_Proteobacteria.c_Alphaproteobacteria.o_Rickettsiales.g_Caedibacter
k_Bacteria.p_Firmicutes.c_Bacilli.o_Bacillales.f_Bacillaceae.g_Halalkalibacillus
k_Bacteria.p_Firmicutes.c_Bacilli.o_Bacillales.f_Listeriaceae.g_Listeria
k_Bacteria.p_Proteobacteria.c_Deltaproteobacteria.o_Myxococcales.f_Nannocystaceae.g_Plesiocystis
k_Bacteria.p_Proteobacteria.c_Deltaproteobacteria.o_Desulfobacterales.f_Desulfobacteraceae.g_Desulfatitalea
k_Bacteria.p_Proteobacteria.c_Betaproteobacteria.o_Burkholderiales.g_Methylibium
k_Bacteria.p_Actinobacteria.c_Actinobacteria.o_Micrococcales.f_Micrococcaceae.g_Citricoccus
k_Bacteria.p_Synergistetes.c_Synergistia.o_Synergistales.f_Synergistaceae.g_Synergistes
k_Bacteria.p_Proteobacteria.c_Gammaproteobacteria.o_Pasteurellales.f_Pasteurellaceae.g_Mannheimia
k_Bacteria.p_Firmicutes.c_Clostridia.o_Thermoanaerobacterales.f_Thermoanaerobacteraceae.g_Carboxydothermus
k_Bacteria.p_Bacteroidetes.c_Bacteroidia.o_Bacteroidales.f_Odoribacteraceae.g_Butyricimonas
k_Bacteria.p_Proteobacteria.c_Betaproteobacteria.o_Rhodocyclales.f_Rhodocyclaceae.g_Methyloversatilis
k_Bacteria.p_Proteobacteria.c_Alphaproteobacteria.o_Rhodobacterales.f_Rhodobacteraceae.g_Planktomarina
k_Bacteria.p_Chloroflexi.c_Ardenticatenia.o_Ardenticatenales.f_Ardenticatenaceae.g_Ardenticatena
k_Bacteria.p_Firmicutes.c_Limnochordia.o_Limnochordales.f_Limnochordaceae.g_Limnochorda
k_Bacteria.p_Cyanobacteria.o_Chroococcales.g_Acaryochloris
k_Bacteria.p_Proteobacteria.c_Alphaproteobacteria.o_Rhodobacterales.f_Hyphomonadaceae.g_Hyphomonas
k_Bacteria.p_Proteobacteria.c_Betaproteobacteria.o_Burkholderiales.f_Oxalobacteraceae.g_Herbaspirillum
k_Bacteria.p_Proteobacteria.c_Alphaproteobacteria.o_Sphingomonadales.f_Erythrobacteraceae.g_Erythrobacter
k_Bacteria.p_Proteobacteria.c_Alphaproteobacteria.o_Rhodospirillales.f_Acetobacteraceae.g_Tanticharoenia
k_Bacteria.p_Proteobacteria.c_Deltaproteobacteria.o_Desulfobacterales.f_Desulfobacteraceae.g_Desulfococcus
k_Bacteria.p_Proteobacteria.c_Gammaproteobacteria.o_Oceanospirillales.f_Halomonadaceae.g_Kushneria
k_Bacteria.p_Bacteroidetes.c_Flavobacteriia.o_Flavobacteriales.f_Flavobacteriaceae.g_Dokdonia
k_Bacteria.p_Chlamydiae.c_Chlamydiia.o_Chlamydiales.f_Waddliaceae.g_Waddlia
k_Bacteria.p_Chloroflexi.c_Chloroflexia.o_Chloroflexales.f_Oscillochloridaceae.g_Oscillochloris
k_Bacteria.p_Proteobacteria.c_Alphaproteobacteria.o_Rhodobacterales.f_Rhodobacteraceae.g_Thalassobius
k_Bacteria.p_Proteobacteria.c_Gammaproteobacteria.o_Oceanospirillales.f_Halomonadaceae.g_Carnimonas
k_Bacteria.p_Proteobacteria.c_Gammaproteobacteria.o_Xanthomonadales.f_Xanthomonadaceae.g_Pseudoxanthomonas
k_Archaea.p_Crenarchaeota.c_Thermoprotei.o_Sulfolobales.f_Sulfolobaceae.g_Acidianus
k_Bacteria.p_Cyanobacteria.o_Oscillatoriales.g_Leptolyngbya
k_Bacteria.p_Proteobacteria.c_Gammaproteobacteria.o_Thiotrichales.f_Piscirickettsiaceae.g_Hydrogenovibrio
k_Bacteria.p_Chloroflexi.c_Anaerolineae.o_Anaerolineales.f_Anaerolineaceae.g_Flexilinea
k_Bacteria.p_Bacteroidetes.c_Flavobacteriia.o_Flavobacteriales.f_Crocinitomicaceae.g_Crocinitomix
k_Bacteria.p_Proteobacteria.c_Gammaproteobacteria.o_Orbales.f_Orbaceae.g_Candidatus_Schmidhempelia
k_Bacteria.p_Firmicutes.c_Clostridia.o_Clostridiales.f_Lachnospiraceae.g_Roseburia
k_Bacteria.p_Cyanobacteria.o_Oscillatoriales.g_Planktothrix
k_Bacteria.p_Acidobacteria.c_Solibacteres.o_Solibacterales.f_Solibacteraceae.g_Candidatus_Solibacter
k_Archaea.p_Euryarchaeota.c_Halobacteria.o_Natrialbales.f_Natrialbaceae.g_Halostagnicola
k_Bacteria.p_Bacteroidetes.c_Bacteroidia.o_Bacteroidales.g_Candidatus_Azobacteroides
k_Bacteria.p_Bacteroidetes.c_Flavobacteriia.o_Flavobacteriales.f_Flavobacteriaceae.g_Riemerella
k_Bacteria.p_Actinobacteria.c_Coriobacteriia.o_Coriobacteriales.f_Atopobiaceae.g_Atopobium
k_Bacteria.p_Planctomycetes.c_Planctomycetia.o_Planctomycetales.f_Planctomycetaceae.g_Zavarzinella
k_Bacteria.p_Proteobacteria.c_Alphaproteobacteria.o_Rhodospirillales.f_Acetobacteraceae.g_Rubritepida
k_Bacteria.p_Proteobacteria.c_Gammaproteobacteria.o_Xanthomonadales.f_Rhodanobacteraceae.g_Dokdonella
k_Bacteria.p_Bacteroidetes.c_Bacteroidia.o_Bacteroidales.f_Prevotellaceae.g_Hallella
k_Bacteria.p_Cyanobacteria.o_Oscillatoriales.g_Oscillatoria
k_Bacteria.p_Proteobacteria.c_Alphaproteobacteria.o_Sphingomonadales.f_Sphingomonadaceae.g_Blastomonas
k_Bacteria.p_Planctomycetes.c_Planctomycetia.o_Candidatus_Brocadiales.f_Candidatus_Brocadiaceae.g_Candidatus_Brocadia
k_Bacteria.p_Proteobacteria.c_Epsilonproteobacteria.o_Campylobacterales.f_Helicobacteraceae.g_Helicobacter
k_Bacteria.p_Bacteroidetes.c_Sphingobacteriia.o_Sphingobacteriales.f_Sphingobacteriaceae.g_Mucilaginibacter
k_Bacteria.p_Cyanobacteria.o_Chroococcales.g_Microcystis
k_Bacteria.p_Proteobacteria.c_Alphaproteobacteria.o_Rhodobacterales.f_Rhodobacteraceae.g_Stappia
k_Bacteria.p_Firmicutes.c_Clostridia.o_Halanaerobiales.f_Halobacteroidaceae.g_Halonatronum
k_Archaea.p_Euryarchaeota.c_Thermoplasmata.o_Thermoplasmatales.f_Picrophilaceae.g_Picrophilus
k_Bacteria.p_Bacteroidetes.c_Flavobacteriia.o_Flavobacteriales.f_Flavobacteriaceae.g_Bergeyella
k_Bacteria.p_Cyanobacteria.o_Oscillatoriales.g_Lyngbya
k_Bacteria.p_Synergistetes.c_Synergistia.o_Synergistales.f_Synergistaceae.g_Pyramidobacter
k_Bacteria.p_Bacteroidetes.c_Flavobacteriia.o_Flavobacteriales.f_Flavobacteriaceae.g_Zhouia
k_Bacteria.p_Acidobacteria.c_Holophagae.o_Holophagales.f_Holophagaceae.g_Holophaga
k_Archaea.p_Euryarchaeota.c_Halobacteria.o_Natrialbales.f_Natrialbaceae.g_Salinarchaeum
k_Bacteria.p_Actinobacteria.c_Actinobacteria.o_Micrococcales.f_Microbacteriaceae.g_Yonghaparkia
k_Bacteria.p_Actinobacteria.c_Actinobacteria.o_Streptosporangiales.f_Streptosporangiaceae.g_Microbispora
k_Bacteria.p_Actinobacteria.c_Coriobacteriia.o_Eggerthellales.f_Eggerthellaceae.g_Adlercreutzia
k_Bacteria.p_Proteobacteria.c_Gammaproteobacteria.o_Legionellales.f_Legionellaceae.g_Tatlockia
k_Archaea.p_Euryarchaeota.c_Halobacteria.o_Haloferacales.f_Haloferacaceae.g_Halorubrum
k_Bacteria.p_Cyanobacteria.o_Chroococcales.g_Geminocystis
k_Bacteria.p_Proteobacteria.c_Alphaproteobacteria.o_Rhizobiales.f_Bradyrhizobiaceae.g_Salinarimonas
k_Bacteria.p_Chlamydiae.c_Chlamydiia.o_Chlamydiales.f_Simkaniaceae.g_Simkania
k_Bacteria.p_Proteobacteria.c_Gammaproteobacteria.o_Enterobacteriales.f_Enterobacteriaceae.g_Candidatus_Regiella
k_Bacteria.p_Thermodesulfobacteria.c_Thermodesulfobacteria.o_Thermodesulfobacteriales.f_Thermodesulfobacteriaceae.g_Thermodesulfobacterium
k_Bacteria.p_Firmicutes.c_Clostridia.o_Clostridiales.f_Lachnospiraceae.g_Anaerostipes
k_Bacteria.p_Proteobacteria.c_Gammaproteobacteria.o_Oceanospirillales.f_Halomonadaceae.g_Halomonas
k_Bacteria.p_Proteobacteria.c_Gammaproteobacteria.o_Nevskiales.f_Sinobacteraceae.g_Steroidobacter
k_Bacteria.p_Proteobacteria.c_Alphaproteobacteria.o_Rhizobiales.f_Hyphomicrobiaceae.g_Pelagibacterium
k_Bacteria.p_Bacteroidetes.c_Chitinophagia.o_Chitinophagales.f_Chitinophagaceae.g_Niabella
k_Bacteria.p_Proteobacteria.c_Betaproteobacteria.o_Neisseriales.f_Chromobacteriaceae.g_Chitinilyticum
k_Bacteria.p_Proteobacteria.c_Alphaproteobacteria.o_Rhizobiales.f_Beijerinckiaceae.g_Beijerinckia
k_Bacteria.p_Cyanobacteria.o_Chroococcales.g_Cyanothece
k_Bacteria.p_Proteobacteria.c_Alphaproteobacteria.o_Rhizobiales.f_Methylobacteriaceae.g_Microvirga
k_Bacteria.p_Actinobacteria.c_Acidimicrobiia.o_Acidimicrobiales.f_Acidimicrobiaceae.g_Ilumatobacter
k_Bacteria.p_Deferribacteres.c_Deferribacteres.o_Deferribacterales.f_Deferribacteraceae.g_Flexistipes
k_Bacteria.p_Actinobacteria.c_Coriobacteriia.o_Eggerthellales.f_Eggerthellaceae.g_Denitrobacterium
k_Bacteria.p_Proteobacteria.c_Gammaproteobacteria.o_Xanthomonadales.f_Rhodanobacteraceae.g_Frateuria
k_Bacteria.p_Proteobacteria.c_Betaproteobacteria.o_Hydrogenophilales.f_Hydrogenophilaceae.g_Thiobacillus
k_Bacteria.p_Proteobacteria.c_Gammaproteobacteria.o_Chromatiales.f_Chromatiaceae.g_Rheinheimera
k_Bacteria.p_Proteobacteria.c_Alphaproteobacteria.o_Rhizobiales.f_Xanthobacteraceae.g_Azorhizobium
k_Bacteria.p_Proteobacteria.c_Gammaproteobacteria.o_Oceanospirillales.f_Alcanivoracaceae.g_Kangiella
k_Bacteria.p_Proteobacteria.c_Deltaproteobacteria.o_Syntrophobacterales.f_Syntrophorhabdaceae.g_Syntrophorhabdus
k_Bacteria.p_Deinococcus.Thermus.c_Deinococci.o_Thermales.f_Thermaceae.g_Thermus
k_Bacteria.p_Cyanobacteria.o_Chroococcales.g_Aphanocapsa
k_Bacteria.p_Proteobacteria.c_Gammaproteobacteria.o_Vibrionales.f_Vibrionaceae.g_Candidatus_Photodesmus
k_Bacteria.p_Firmicutes.c_Clostridia.o_Clostridiales.f_Oscillospiraceae.g_Oscillibacter
k_Bacteria.p_Proteobacteria.c_Deltaproteobacteria.o_Desulfovibrionales.f_Desulfohalobiaceae.g_Desulfonatronospira
k_Bacteria.p_Actinobacteria.c_Actinobacteria.o_Bifidobacteriales.f_Bifidobacteriaceae.g_Parascardovia
k_Bacteria.p_Bacteroidetes.c_Cytophagia.o_Cytophagales.f_Cyclobacteriaceae.g_Indibacter
k_Bacteria.p_Cyanobacteria.o_Chroococcales.g_Chamaesiphon
k_Bacteria.p_Proteobacteria.c_Alphaproteobacteria.o_Sphingomonadales.f_Sphingomonadaceae.g_Sphingobium
k_Bacteria.p_Proteobacteria.c_Alphaproteobacteria.o_Rhizobiales.f_Hyphomicrobiaceae.g_Prosthecomicrobium
k_Bacteria.p_Bacteroidetes.c_Bacteroidia.o_Bacteroidales.f_Porphyromonadaceae.g_Parabacteroides
k_Bacteria.p_Bacteroidetes.c_Flavobacteriia.o_Flavobacteriales.f_Flavobacteriaceae.g_Maribacter
k_Bacteria.p_Proteobacteria.c_Betaproteobacteria.o_Burkholderiales.f_Alcaligenaceae.g_Brackiella
k_Bacteria.p_Acidobacteria.c_Blastocatellia.g_Chloracidobacterium
k_Bacteria.p_Proteobacteria.c_Betaproteobacteria.o_Burkholderiales.f_Alcaligenaceae.g_Kerstersia
k_Bacteria.p_Proteobacteria.c_Alphaproteobacteria.o_Rhodobacterales.f_Rhodobacteraceae.g_Nereida
k_Bacteria.p_Chloroflexi.c_Anaerolineae.o_Anaerolineales.f_Anaerolineaceae.g_Leptolinea
k_Bacteria.p_Actinobacteria.c_Actinobacteria.o_Micrococcales.f_Microbacteriaceae.g_Leifsonia
k_Bacteria.p_Proteobacteria.c_Gammaproteobacteria.o_Enterobacteriales.f_Enterobacteriaceae.g_Lelliottia
k_Bacteria.p_Proteobacteria.c_Gammaproteobacteria.o_Alteromonadales.f_Colwelliaceae.g_Colwellia
k_Bacteria.p_Proteobacteria.c_Epsilonproteobacteria.o_Campylobacterales.f_Campylobacteraceae.g_Arcobacter
k_Bacteria.p_Actinobacteria.c_Actinobacteria.o_Micrococcales.f_Microbacteriaceae.g_Agrococcus
k_Bacteria.p_Proteobacteria.c_Gammaproteobacteria.o_Vibrionales.f_Vibrionaceae.g_Grimontia
k_Bacteria.p_Bacteroidetes.c_Flavobacteriia.o_Flavobacteriales.f_Flavobacteriaceae.g_Sediminibacter
k_Bacteria.p_Deferribacteres.c_Deferribacteres.o_Deferribacterales.f_Deferribacteraceae.g_Deferribacter
k_Bacteria.p_Proteobacteria.c_Betaproteobacteria.o_Burkholderiales.g_Thiomonas
k_Bacteria.p_Proteobacteria.c_Betaproteobacteria.o_Rhodocyclales.f_Rhodocyclaceae.g_Thauera
k_Bacteria.p_Actinobacteria.c_Actinobacteria.o_Actinomycetales.f_Actinomycetaceae.g_Varibaculum
k_Bacteria.p_Bacteroidetes.c_Flavobacteriia.o_Flavobacteriales.f_Flavobacteriaceae.g_Bizionia
k_Bacteria.p_Bacteroidetes.c_Bacteroidia.o_Bacteroidales.f_Marinilabiliaceae.g_Anaerophaga
k_Bacteria.p_Cyanobacteria.c_Gloeobacteria.o_Gloeobacterales.g_Gloeobacter
k_Archaea.p_Euryarchaeota.c_Methanomicrobia.o_Methanosarcinales.f_Methanosarcinaceae.g_Methanosarcina
k_Bacteria.p_Actinobacteria.c_Thermoleophilia.o_Solirubrobacterales.f_Conexibacteraceae.g_Conexibacter
k_Bacteria.p_Tenericutes.c_Mollicutes.o_Mycoplasmatales.f_Mycoplasmataceae.g_Ureaplasma
k_Bacteria.p_Actinobacteria.c_Actinobacteria.o_Micrococcales.f_Microbacteriaceae.g_Microbacterium
k_Archaea.p_Euryarchaeota.c_Halobacteria.o_Haloferacales.f_Haloferacaceae.g_Halolamma
k_Bacteria.p_Proteobacteria.c_Epsilonproteobacteria.o_Campylobacterales.f_Campylobacteraceae.g_Sulfurospirillum
k_Bacteria.p_Spirochaetes.c_Spirochaetia.o_Spirochaetales.f_Spirochaetaceae.g_Spirochaeta
k_Bacteria.p_Actinobacteria.c_Actinobacteria.o_Micrococcales.f_Microbacteriaceae.g_Cryobacterium
k_Bacteria.p_Actinobacteria.c_Actinobacteria.o_Corynebacteriales.f_Gordoniaceae.g_Gordonia
k_Bacteria.p_Planctomycetes.c_Planctomycetia.o_Planctomycetales.f_Planctomycetaceae.g_Rubinisphaera
k_Bacteria.p_Actinobacteria.c_Actinobacteria.g_Thermobispora
k_Bacteria.p_Actinobacteria.c_Actinobacteria.o_Glycomycetales.f_Glycomycetaceae.g_Glycomyces
k_Bacteria.p_Proteobacteria.c_Betaproteobacteria.o_Burkholderiales.f_Comamonadaceae.g_Ottowia
k_Bacteria.p_Proteobacteria.c_Alphaproteobacteria.o_Rhizobiales.f_Xanthobacteraceae.g_Ancylobacter
k_Bacteria.p_Firmicutes.c_Clostridia.o_Clostridiales.f_Ruminococcaceae.g_Faecalibacterium
k_Bacteria.p_Firmicutes.c_Bacilli.o_Bacillales.f_Bacillaceae.g_Paucisalibacillus
k_Bacteria.p_Proteobacteria.c_Betaproteobacteria.o_Burkholderiales.f_Burkholderiaceae.g_Polynucleobacter
k_Bacteria.p_Proteobacteria.c_Alphaproteobacteria.o_Rhodospirillales.f_Rhodospirillaceae.g_Caenispirillum
k_Archaea.p_Euryarchaeota.c_Methanomicrobia.o_Methanomicrobiales.f_Methanoregulaceae.g_Methanoregula
k_Archaea.p_Crenarchaeota.c_Thermoprotei.o_Thermoproteales.f_Thermofilaceae.g_Thermofilum
k_Bacteria.p_Proteobacteria.c_Betaproteobacteria.o_Sulfuricellales.f_Sulfuricellaceae.g_Sulfuricella
k_Bacteria.p_Proteobacteria.c_Deltaproteobacteria.o_Bdellovibrionales.f_Bacteriovoracaceae.g_Bacteriovorax
k_Bacteria.p_Bacteroidetes.c_Chitinophagia.o_Chitinophagales.f_Chitinophagaceae.g_Niastella
k_Bacteria.p_Actinobacteria.c_Actinobacteria.o_Micrococcales.f_Micrococcaceae.g_Yaniella
k_Bacteria.p_Proteobacteria.c_Gammaproteobacteria.o_Enterobacteriales.f_Enterobacteriaceae.g_Citrobacter
k_Bacteria.p_Proteobacteria.c_Deltaproteobacteria.o_Myxococcales.f_Polyangiaceae.g_Sorangium
k_Archaea.p_Euryarchaeota.c_Methanomicrobia.o_Methanosarcinales.f_Methermicoccaceae.g_Methermicoccus
k_Bacteria.p_Aquificae.c_Aquificae.o_Aquificales.f_Aquificaceae.g_Hydrogenobacter
k_Bacteria.p_Synergistetes.c_Synergistia.o_Synergistales.f_Synergistaceae.g_Cloacibacillus
k_Bacteria.p_Firmicutes.c_Clostridia.o_Clostridiales.f_Lachnospiraceae.g_Sellimonas
k_Archaea.p_Thaumarchaeota.g_Candidatus_Nitrosotenuis
k_Bacteria.p_Rhodothermaeota.c_Balneolia.o_Balneolales.f_Balneolaceae.g_Gracilimonas
k_Archaea.p_Crenarchaeota.c_Thermoprotei.o_Thermoproteales.f_Thermoproteaceae.g_Caldivirga
k_Bacteria.p_Firmicutes.c_Clostridia.o_Clostridiales.f_Clostridiales_Family_XIII._IncertaeSedis.g_Casaltella
k_Archaea.p_Crenarchaeota.c_Thermoprotei.o_Desulfurococcales.f_Desulfurococcaceae.g_Thermosphaera
k_Bacteria.p_Proteobacteria.c_Betaproteobacteria.o_Neisseriales.f_Chromobacteriaceae.g_Deefgea
k_Bacteria.p_Proteobacteria.c_Alphaproteobacteria.o_Rhizobiales.f_Phyllobacteriaceae.g_Aquamicrobium
k_Bacteria.p_Deferribacteres.c_Deferribacteres.o_Deferribacterales.f_Deferribacteraceae.g_Geovibrio
k_Bacteria.p_Firmicutes.c_Clostridia.o_Clostridiales.f_Ruminococcaceae.g_Subdoligranulum

1.-80. (canceled)

81. A method for creating a diagnostic model based on non-mammalian, microbial features to diagnose cancer in a subject comprising:
(a) detecting a microbial presence in a biological sample from one or more subjects;
(b) identifying and removing contaminated microbial features of the microbial presence while retaining other decontaminated microbial features, thereby producing a decontaminated microbial presence;
(c) determining a shared pattern of the decontaminated microbial presence among one or more subjects;
(d) forming an association between the shared pattern of the decontaminated microbial presence and type of cancer present in the one or more subjects; and
(e) summarizing the association in a diagnostic model configured to diagnose cancer in a biological sample different than the biological sample from the one or more subjects.

82. The method of claim 81, wherein the microbial presence comprises a microbial abundance of the biological sample from the one or more subjects.

83. The method of claim 81, wherein the contaminated and decontaminated microbial features comprise taxonomic assignment of the microbial presence.

84. The method of claim 82, wherein the diagnostic model utilizes the microbial presence or abundance from one or more of the following non-mammalian domains of life: viruses, bacteria, fungi, or archaea.

85. The method of claim 81, wherein the diagnostic model diagnoses one or more cancers, one or more subtypes of cancer, or any combination thereof in the subject.

86. The method of claim 81, wherein the diagnostic model is configured to predict a stage of cancer, predict cancer prognosis, diagnose a type of cancer at a low-stage (stage I or stage II), diagnose a mutation status of one or more cancers, predict a future immunotherapy response, select an optimal therapy, or any combination thereof for the subject.

87. The method of claim 81, wherein the diagnostic model diagnoses one or more stage I or stage II cancers.

88. The method of claim 81, wherein the diagnostic model simultaneously discriminates among one or more cancer types to diagnose a specific cancer type in the subject.

89. The method of claim 81, wherein the diagnostic model diagnoses one or more of the following: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, or uveal melanoma.

90. The method of claim 81, wherein the diagnostic model diagnoses one or more of the following cancer types outside the intestine: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, or uveal melanoma.

91. The method of claim 81, wherein the diagnostic model uses a decontamination pipeline to identify the contaminated microbial features.

92. The method of claim 81, wherein step (b) improves performance of the diagnostic model.

93. The method of claim 81, wherein step (b) is omitted.

94. The method of claim 81, wherein the diagnostic model comprises a machine learning model, regularized machine learning model, ensemble of machine learning models, or any combination thereof.

95. The method of claim 81, wherein the one or more subjects comprise non-human mammal or human subjects.

96. The method of claim 81, wherein the biological sample comprises a tissue sample, liquid biopsy, whole blood biopsy, or any combination thereof.

97. The method of claim 96, wherein the whole blood biopsy comprises one or more of the following: plasma, white blood cells, red blood cells, or platelets.

98. The method of claim 81, wherein the diagnostic model comprises one or more of the following mammalian feature types in addition to the non-contaminant microbial features to diagnose cancer in a subject: cell-free tumor DNA, cell-free tumor RNA, exosomal-derived tumor DNA, exosomal-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, or methylation patterns of circulating tumor cell derived RNA.

99. The method of claim 81, wherein the diagnostic model diagnosis a bodily location of the cancer.

100. A method of providing a diagnosis of cancer in a subject comprising:
(a) detecting microbial presence in a biological sample from the subject;
(b) identifying and removing contaminated microbial features of the microbial presence while retaining decontaminated microbial features, thereby producing a decontaminated microbial presence;
(c) determining whether the decontaminated microbial presence is similar to or different than a microbial presence in one or more biological samples from healthy or diseased subjects; and
(d) correlating the decontaminated microbial presence with a known microbial presence for one or more cancer types, thereby providing the diagnosis of the cancer.

101. The method of claim 100, wherein the microbial presence comprises microbial abundance of the biological sample from the subject.

102. The method of claim 100, wherein the diagnosis of the cancer comprises the bodily location of the cancer.

103. The method of claim 101, wherein correlating comprises comparing lists of the decontaminated microbial presence or abundances of the subject and the microbial presence or abundance of the healthy or diseased subjects with a statistical model to associate the decontaminated microbial presence with the one or more cancer types.

104. The method of claim 100, wherein the contaminated and decontaminated microbial features comprise taxonomic assignment of the microbial presence.

105. The method of claim 101, wherein the microbial presence or abundance comprises one or more of the following non-mammalian domains of life: viruses, bacteria, fungi, or archaea.

106. The method of claim 100, wherein the diagnosis comprises the presence or absence of one or more cancers, one or more subtypes of cancer, or any combination thereof.

107. The method of claim 100, wherein the diagnosis comprises the stage of cancer, cancer prognosis, or any combination thereof in the subject.

108. The method of claim 100, wherein the diagnosis of the cancer comprises a type of cancer at a low-stage (stage I or stage II) tumor.

109. The method of claim 100, wherein the diagnosis comprises the mutation status of one or more cancers in the subject.

110. The method of claim 100, wherein the diagnosis comprises the anticipated response to immunotherapy of the subject.

111. The method of claim 100, further comprising selecting an optimal treatment for the cancer of the subject, wherein the optimal treatment comprises one or more of the following: a small molecule, a biologic, an engineered host-derived cell type or types, a probiotic, an engineered bacterium, a natural-but-selective virus, an engineered virus, or a bacteriophage.

112. The method of claim 100, wherein the diagnosis comprises diagnosing one or more of the following cancer types: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, or uveal melanoma.

113. The method of claim 100, wherein the diagnosis comprises diagnosing one or more of the following cancer types located outside the intestine: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, or uveal melanoma.

114. The method of claim 100, wherein step (b) improves diagnostic accuracy of the diagnosis of the cancer.

115. The method of claim 100, wherein step (b) is omitted.

116. The method of claim 100, wherein the subject comprises a non-human mammal or human.

117. The method of claim 100, wherein the diagnosis comprises mammalian features in addition to the decontaminated microbial presence to provide the diagnosis of one or more cancers in the subject.

118. The method of claim 117, wherein the mammalian features comprise one or more of the following: cell-free tumor DNA, cell-free tumor RNA, exosomal-derived tumor DNA, exosomal-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, or methylation patterns of circulating tumor cell derived RNA, methylation patterns of circulating tumor cell derived RNA.

119. The method of claim 100, wherein the biological sample comprises a tissue sample, liquid biopsy, whole blood biopsy, or any combination thereof.

120. The method of claim 100, wherein the biological sample is one or more constituents of whole blood, including one or more of the following: plasma, white blood cells, red blood cells, or platelets.

121. The method of claim 101, wherein the microbial presence or abundance is measured by ecological shotgun sequencing, quantitative polymerase chain reaction, immunohistochemistry, in situ hybridization, flow cytometry, host whole genome sequencing, host transcriptomic sequencing, cancer whole genome sequencing, cancer transcriptomic sequencing, or any combination thereof.

122. The method of claim 101, wherein the microbial presence or abundance is measured using amplification of one or more of the following nucleic acid regions of microbial origin: V1, V2, V3, V4, V5, V6, V7, V8, or V9 variable domain region of 16S rRNA; or the internal transcribed spacer (ITS) region of the 18S rRNA.

123. The method of claim 101, wherein the microbial presence or abundance is detected by nucleic acid measurement that targets microbial DNA, RNA, or any combination thereof, wherein the measurement that targets microbial DNA, RNA, or any combination thereof occurs simultaneously with the measurement of host DNA, RNA, or any combination thereof.

124. A method for treating a cancer in a subject based on non-mammalian microbial presence comprising:
(a) detecting microbial presence in a biological sample from the subject with cancer;
(b) identifying and removing contaminated microbial features of the microbial presence while retaining other decontaminated microbial features, thereby producing a decontaminated microbial presence;
(c) forming an association between the decontaminated microbial presence and the cancer present in the subject; and
(d) administering to the subject a therapeutically effective amount of a treatment determined by the association between the decontaminated microbial presence and the cancer thereby treating the cancer.

125. The method of claim 124, wherein the microbial presence comprises microbial abundance in the biological sample from the one or more subjects.

126. The method of claim 124, wherein the microbial features comprise taxonomic assignment of the microbial presence.

127. The method of claim 124, wherein step (b) is omitted.

128. The method of claim 124, wherein the subject comprises a non-human mammal or human.

129. The method of claim 124, wherein the biological sample comprises a tissue sample, liquid biopsy, whole blood biopsy, or any combination thereof.

130. The method of claim 124, wherein the biological sample comprises one or more constituents of whole blood, including one or more of the following: plasma, white blood cells, red blood cells, or platelets.

131. The method of claim 125, wherein the treatment repurposes an existing medication, which may or may not have been originally approved for targeting cancer, to improve overall therapeutic efficacy by exploiting microbial presence or abundance information.

132. The method of claim 124, wherein the treatment comprises a small molecule, a biologic, a probiotic, a virus, a bacteriophage, immunotherapy, broad spectrum antibiotic, or any combination thereof.

133. The method of claim 132, wherein the probiotic comprises an engineered bacterium strain or ensemble of engineered bacteria.

134. The method of claim 124, wherein the treatment comprises an adjuvant given in combination with a primary treatment against the cancer to improve the efficacy of the primary treatment.

135. The method of claim 124, wherein the treatment comprises adoptive cell transfer to target microbial antigens associated with the cancer or cancer microenvironment.

136. The method of claim 124, wherein the treatment comprises a cancer vaccine that exploits microbial antigens associated with the cancer or cancer microenvironment.

137. The method of claim 124, wherein the treatment comprises a monoclonal antibody against microbial antigens associated with the cancer or cancer microenvironment.

138. The method of claim 124, wherein the treatment comprises an antibody-drug-conjugate designed to at least partially target microbial antigens associated with the cancer or cancer microenvironment.

139. The method of claim 124, wherein the treatment comprises a multi-valent antibody, antibody fragment, or antibody derivative thereof designed to at least partially target one or more microbial antigens associated with the cancer or cancer microenvironment.

140. The method of claim 124, wherein the treatment comprises a targeted antibiotic against a particular kind of microbe or class of functionally or biologically similar microbes.

141. The method of claim 124, wherein two or more of the following treatment types are combined such that at least one type exploits the cancer microbial presence or abundance to enhance overall therapeutic efficacy: small molecules, biologics, engineered host-derived cell types, probiotics, engineered bacteria, natural-but-selective viruses, engineered viruses, and bacteriophages.

142. The method of claim 124, wherein the association comprises mammalian features in addition to the decontaminated microbial presence.

143. The method of claim 142, wherein the mammalian features comprise one or more of the following: cell-free tumor DNA, cell-free tumor RNA, exosomal-derived tumor DNA, exosomal-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, or methylation patterns of circulating tumor cell derived RNA, methylation patterns of circulating tumor cell derived RNA.

144. The method of claim 124, wherein the association comprises the bodily location of the cancer in addition to the decontaminated microbial presence.
